(19) United States
(12) Patent Application Publication
(10) Pub. No.: US 2006/0270656 A1
(43) Pub. Date: Nov. 30, 2006

(54) Substituted piperazines of azepines, oxazepines and thiazepines

(75) Inventors: John Xiaoqiang He, Fishers, IN (US); Vincent Patrick Rocco, Indianapolis, IN (US); John Mehnert Schaus, Zionsville, IN (US); Fionna Mitchell Martin, Hampshire (GB); William Martin Owton, Basingstoke (GB); David Edward Tupper, Basingstoke (SG)

Correspondence Address: ELI LILLY & COMPANY, PATENT DIVISION, P.O. BOX 6288, INDIANAPOLIS, IN, 46206-6288, US

(73) Assignee: ELI LILLY AND COMPANY

(21) Appl. No.: 10/569,244

(22) PCT Filed: Sep. 7, 2004
(86) PCT No.: PCT/US04/25610

§ 371 Date: Feb. 21, 2006

(30) Foreign Application Priority Data
Sep. 9, 2003 (US) 60501473

(51) Int. Cl. A61K 31/554; A61K 31/55; A61K 31/553; C07D 498/02; C07D 491/02

(52) U.S. Cl. 514/211.13; 514/215; 540/551; 540/586

(57) ABSTRACT

Described herein are antipyschotic compounds of formula (I) 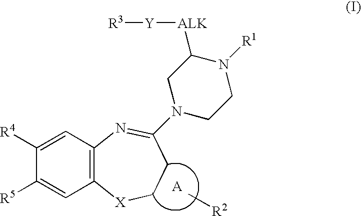
wherein: 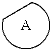 is an optionally benzo-fused five or six member aromatic ring having zero to three hetero atoms independently selected from N, O, and S;
R¹ is hydrogen, (C₁₋₆) fluoroalkyl, (C₃₋₆) cycloalkyl, or (C₁₋₄) alkyl, wherein the (C₁₋₄) alkyl is unsubstituted or substituted with hydroxy, methoxy, ethoxy, OCH₂CH₂OH, —CN, imidazolidin-2-one, phenyl, or tetrazole wherein tetrazole is unsubstituted or substituted with (C₁₋₄) alkyl;
R² is H, halogen, (C₁₋₆) fluoroalkyl, (C₃₋₆) cycloalkyl, OR⁶, SR⁶, NO₂, CN, COR⁶, C(O)OR⁶, C(OH)R⁶, CONR⁷R⁸, phenyl or (C₁₋₆) alkyl, wherein the (C₁₋₆) alkyl is unsubstituted or substituted with a hydroxy;
R³ is hydrogen, (C₁₋₆)fluoroalkyl , (C₃₋₆) cycloalkyl, (C₂₋₆) alkenyl, phenyl, monocyclic heteroaromatic, bicyclic heteroaromatic, or (C₁₋₄)alkyl wherein (C₁₋₄) alkyl is unsubstituted or substituted with a phenyl;
R⁴ and R⁵ are independently selected from hydrogen, halogen, (C₁₋₆) alkyl, (C₁₋₆) fluoroalkyl, OR⁹, SR⁹, NO₂, CN, or COR⁹;
R⁶ is hydrogen, (C₁₋₆) fluoroalkyl, or (C₁₋₆) alkyl;
R⁷ and R⁸ are independently hydrogen, or (C₁₋₆) alkyl;
R⁹ is hydrogen, (C₁₋₆) fluoroalkyl, (C₁₋₆) alkyl;
Alk is (C₁₋₄) alkylene unsubstituted or substituted with a hydroxy;
Y is oxygen, sulfur, SO₂, or a bond;
X is CH₂, C═O, S, O, or SO₂;
Z is hydrogen, halogen, (C₁₋₆) alkyl, (C₁₋₆)fluoroalkyl, —OH, (C₁₋₆) alkoxy, (C₁₋₆) fluoroalkoxy, (C₁₋₆) alkylthio, (C₁₋₆) acyl, (C₁₋₄)alkylsulfonyl, —OCF₃, —NO₂, —CN, carboxamido which may be substituted on the nitrogen by one or two (C₁₋₄) alkyl groups, and —NH₂ in which one of the hydrogens may be replaced by a (C₁₋₄) alkyl group and the other hydrogen may be replaced by either a (C₁₋₄) alkyl group, a (C₁₋₆) acyl group, or a (C₁₋₄) alkylsulfonyl group;
the phenyl of R¹, R² or R³ is independently unsubstituted or substituted with one to three substituents independently selected from Z;
the monocyclic heteroaromatic of R³ is unsubstituted or substituted with one to three substituents independently selected from Z;
the bicyclic heteroaromatic of R³ is unsubstituted or substituted with one to three substituents independently selected from Z; and salts, solvates, and crystal forms thereof.

Also described are the use of the compounds of formula (I) as antagonists of the dopamine D₂ receptor and as agents for the treatment of psychosis and bipolar disorders, and pharmaceutical formulations of the compounds of formula (I).

### BACKGROUND OF THE INVENTION

Currently there are many drugs available for the treatment of disorders of the central nervous system. Among these drugs is a category known as antipsychotics which are used for treating serious mental conditions such as schizophrenia and schizophreniform illnesses. Currently available treatments for such conditions are often associated with undesirable adverse events. As such, there remains a need for new compounds that control or eliminate the symptoms of such mental conditions with improved adverse event profiles.

Patients suffering from schizophrenia, a condition of unknown etiology, exhibit a group of both positive and negative symptoms. Positive symptoms include delusions, hallucinations, disordered thoughts, and disorganized speech, while negative symptoms include flat affect, anhedonia, social withdrawal, emotional detachment, cognitive deficits, and poverty of speech. Not only does schizophrenia cause personal suffering by the patient, it also severely affects the patient's occupational and social functions, so that often the patient must be institutionalized, which results in a high cost to society.

A leading hypothesis suggests that the positive symptoms of schizophrenia can be effectively treated by compounds that act as antagonists at certain dopamine receptors. Currently, five principal dopamine receptors (D₁-D₅) have been identified. Antipsychotic efficacy has been most closely associated with blockade of the D₂ class of dopamine receptors. One class of antipsychotic agents known as “typical” antipsychotic agents (eg. haloperidol) are effective in controlling the positive symptoms of schizophrenia However, they do not adequately treat the negative symptoms and are associated with significant adverse events, principally hyperprolactinemia, tardive dyskinesia, and extrapyramidal side effects (EPS).

One approach to developing better antipsychotic agents, involves the identification of compounds that combine D₂ receptor blockade with actions at other receptors. One such agent is clozapine.

Clozapine was the first drug identified as an “atypical” antipsychotic, i.e., a drug effective in treating both the positive and negative symptoms of schizophrenia. Additionally, it has a decreased propensity to induce EPS, hyprolactinemia, and tardive dyskinesia seen with classical, “typical” antipsychotics. Although clozapine is an effective drug, its utility in treating schizophrenia has been limited because of the clinical observation that 1-2% of treated patients developed a potentially fatal blood disorder, agranulocytosis. More recently, olanzapine has been widely accepted as an atypical antipsychotic with relatively few adverse events.

However, weight gain has been observed during treatment with many of the atypical antipsychotic compounds (Wetterling, “Body Weight Gain with Atypical Antipsychotics, A Comparative Review”, Drug Safety 24, 59-73 (2001); Wirshing, et al, “Novel Antipsychotics: Comparison of Weight Gain Liabilities”, J. Clin. Psychiatry 60, 358-363 (1999); Allison, et al, “Antipsychotic-Induced Weight Gain: A Comprehensive Research Synthesis” Am. J. Psychiatry 156, 1686-1696 (1999); Ganguli, R. Weight gain associated with antipsychotic drugs. J. Clin. Psychiatry 60(suppl. 2), 20-24, (1999). Drugs with the clinical efficacy and safety profile of the atypical antipsychotics but with decreased propensity to induce weight gain would represent improved agents for the treatment of schizophrenia, bipolar disorder, and related disorders.

Atypical antipsychotics like clozapine and olanzapine are D₂ receptor antagonists but also interact with other neurotransmitter receptors, including other subtypes for dopamine, and certain receptor subclasses for serotonin, norepinephrine, histamine, and acetylcholine. It is believed that some of these additional receptor activities are responsible for the improved efficacy of the atypical antipsychotics and the adverse events of these agents may be mediated by interactions with others. In particular, it has been suggested that the weight gain effects of the atypical antipsychotics may be due to the blockade of the histamine H1 receptor (Wetterling, “Body Weight Gain with Atypical Antipsychotics, A Comparative Review”, Drug Safety 24 59-73 (2001); Wirshing, et al, “Novel Antipsychotics: Comparison of Weight Gain Liabilities” J. Clin. Psychiatry 60, 358-363 (1999); Kroeze, et al. “H1 Histamine Receptor Affinity Predicts Short-Term Weight Gain for Typical and Atypical Antipsychotic Drugs”, Neuropsychopharmacology 28, 519-526 (2003); Orthen-Gambill, N. Antihistaminic drugs increase feeding, while histidine suppresses feeding in rats. Pharmacol. Biochem. Behav. 31, 81-86, (1988). Hence, the development of atypical antipsychotics with decreased affinity for the histamine H₁ receptor represents one mechanism for identifying antipsychotics with improved adverse event profiles.

The present invention provides antipsychotic compounds and methods of using those compounds to treat psychotic disorders, in particular, schizophrenia and mood disorders, such as bipolar disorders. These compounds offer certain improvements and advantages over the currently available antipsychotic agents, as for example, but not limited to, improved adverse event profiles. In particular, many of the compounds of this invention have reduced propentsity to cause weight gain because of their decreased affinity for the H₁ receptor.

### BRIEF SUMMARY OF THE INVENTION

One aspect of the present invention provides compounds of formula (I): 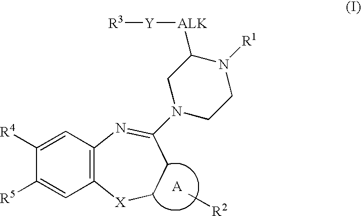
wherein:

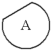

is an optionally benzo-fused five or six member aromatic ring having zero to three hetero atoms independently selected from N, O, and S;

R¹ is hydrogen, (C₁₋₆) fluoroalkyl, (C₃₋₄) cycloalkyl, or (C₁₋₄) alkyl, wherein the (C₁₋₄) alkyl is unsubstituted or substituted with hydroxy, methoxy, ethoxy, OCH₂CH₂OH, —CN, imidazolidin-2-one, phenyl, or tetrazole, wherein tetrazole is unsubstituted or substituted with (C₁₋₄)alkyl;

R² is H, halogen, C₁₋₆) fluoroalkyl, (C₃₋₆) cycloalkyl, OR⁶, SR⁶, NO₂, CN, COR⁶, C(O)OR⁶, C(OH)R⁶, CONR⁷R⁸, phenyl, or (C₁₋₆) alkyl, wherein the (C₁₋₆) alkyl is unsubstituted or substituted with a hydroxy;

R³ is hydrogen, (C₁₋₆) fluoroalkyl, (C₃₋₆) cycloalkyl, (C₂₋₆) alkenyl, phenyl, monocyclic heteroaromatic, bicyclic aromatic, or (C₁₋₄)alkyl, wherein (C₁₋₄)alkyl is unsubstituted or substituted with a phenyl;

R⁴ and R⁵ are independently selected from hydrogen, halogen, (C₁₋₆) alkyl, (C₁₋₆) fluoroalkyl, OR⁹, SR⁹, NO₂, CN, or COR⁹;

R⁶ is hydrogen, (C₁₋₆) alkyl, or (C₁₋₆) fluoroalkyl;

R⁷ and R⁸ are independently hydrogen, or (C₁₋₆) alkyl;

R⁹ is hydrogen, (C₁₋₆) alkyl, or (C₁₋₆) fluoroalkyl;

Alk is (C₁₋₄) alkylene unsubstituted or substituted with a hydroxy;

Y is oxygen, sulfur, S═O, SO₂, or a bond;

X is CH₂, C═O, S, O, or SO₂;

Z is hydrogen, halogen, (C₁₋₆) alkyl, (C₁₋₆) fluoroalkyl, —OH, (C₁₋₆) alkoxy, (C₁₋₆) fluoroalkoxy, (C₁₋₆) alkylthio, (C₁₋₆) acyl, (C₁₋₄)alkylsulfonyl, —OCF₃, —NO₂, —CN, carboxamido which may be substituted on the nitrogen by one or two (C₁₋₄) alkyl groups, and —NH₂ in which one of the hydrogens may be replaced by a (C₁₋₄) alkyl group and the other hydrogen may be replaced by either a (C₁₋₄) alkyl group, a (C₁₋₆) acyl group, or a (C₁₋₄) alkylsulfonyl group;

the phenyl of R¹, R² or R³ are independently unsubstituted or substituted with one to three substituents independently selected from Z;

the monocyclic heteroaromatic of R³ is unsubstituted or substituted with one to three substituents independently selected from Z;

the bicyclic aromatic of R³ is unsubstituted or substituted with one to three substituents independently selected from Z;

and salts, solvates, and crystal forms thereof.

Also preferred among the compounds of formula (I) are those wherein the stereo configuration is “S” about the carbon of the piperazine group bound to Alk. More preferred are those “S”-configuration compounds wherein Alk is (C₂₋₄) alkylene when Y is equal to O, S, or a bond. More preferred are those “S”-configuration compounds wherein Alk is methylene and Y is a bond.

Also preferred among the compounds of formula (I) are those wherein the stereo configuration is “R” about the carbon of the piperazine group bound to Alk. More preferred are those “R”-configuration compounds wherein Alk is methylene and Y is O or S.

Also preferred among the compounds of formula (I) are those wherein Alk is —CH₂—, —CH₂CH₂—, —CH₂CH₂CH₂—, —CH₂CH(CH₃)— or —CH₂C(CH₃)₂—. More preferred are compounds wherein Alk is —CH₂CH₂CH₂— or —CH₂CH₂—.

Also preferred among the compounds of formula (I) are those wherein X is O, S or CH₂.

Also preferred among the compounds of formula (I) are those wherein Y is O or a bond.

Also preferred among the compounds of formula (I) are those wherein R¹ is (C₁₋₄) alkyl. More preferred are compounds wherein R¹ is methyl.

Also preferred among the compounds of formula (I) are those wherein R² is (C₁₋₆) alkyl;

Also preferred among the compounds of formula (I) are those wherein R³ is phenyl or (C₁₋₄) alkyl. More preferred are compounds wherein R³ is phenyl, methyl or ethyl.

Also preferred among the compounds of formula (I) are those wherein R⁴ and R⁵ are independently selected from hydrogen and halogen.
Also preferred among the compounds of formula (I) are those wherein 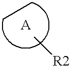
is 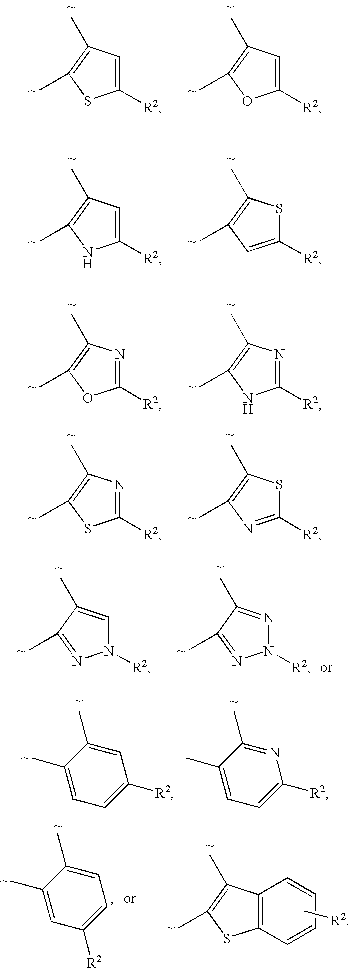
More preferred are compounds wherein

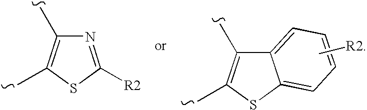

Another aspect of the invention provides a pharmaceutical composition comprising an effective amount of a compound of formula (I) in association with a pharmaceutically acceptable carrier, diluent or excipient.

Another aspect of the invention provides a pharmaceutical composition comprising a compound of formula (I) in an amount effective to antagonize D₂ receptor stimulation, and a pharmaceutically acceptable carrier, diluent or excipient.

Another aspect of the invention provides a pharmaceutical composition comprising a compound of formula (I) in an amount effective to antagonize 5-HT_{2A} receptor stimulation, and a pharmaceutically acceptable carrier, diluent or excipient.

Another aspect of the invention provides a pharmaceutical composition, comprising a compound of formula (I) in an amount effective to antagonize 5-HT₆ receptor stimulation, and a pharmaceutically acceptable carrier, diluent or excipient.

Another aspect of the invention provides a method for antagonizing dopamine receptor D₂, comprising administering to a mammal an effective amount of a compound of formula (I).

Another aspect of the invention provides a method for antagonizing a 5-HT_{2A} receptor, comprising administering to a mammal an effective amount of a compound of formula (I).

Another aspect of the invention provides a method for antagonizing a 5-HT₆ receptor, comprising administering to a mammal an effective amount of a compound of formula (I).

Another aspect of the invention provides a method for treating a psychotic disorder, comprising administering to a mammal in need thereof an effective amount of a compound of formula (I). In a preferred embodiment, the psychotic disorder is schizophrenia, schizophreniform, or schizoaffective disorder.

Another aspect of the invention provides a compound of formula (I) for use in treating a psychotic disorder. In a preferred embodiment, the psychotic disorder is schizophrenia, schizophreniform, or schizoaffective disorder.

Another aspect of the invention provides use of a compound of formula (I) for the manufacture of a medicament for the treatment of a psychotic disorder. In a preferred embodiment, the psychotic disorder is schizophrenia, schizophreniform, or schizoaffective disorder.

Another aspect of the invention provides a method for treating a mood disorder, comprising administering to a mammal in need thereof an effective amount of a compound of formula (I). In a preferred embodiment, the mood disorder is a bipolar disorder. In a more preferred embodiment, the bipolar disorder is bipolar I disorder or bipolar II disorder.

Another aspect of the invention provides a compound of formula (I) for use in treating a mood disorder. In a prefeired embodiment, the mood disorder is a bipolar disorder. In a more preferred embodiment, the bipolar disorder is bipolar I disorder or bipolar II disorder.

Another aspect of the invention provides use of a compound of formula (I) for the manufacture of a medicament for the treatment of a mood disorder. In a preferred embodiment, the mood disorder is a bipolar disorder. In a more preferred embodiment, the bipolar disorder is bipolar I disorder or bipolar II disorder.

Another aspect of the invention involves improved adverse event profiles (e.g., reduced weight gain) over currently available antipsychotic agents and/or better dopamine D₂ binding

### DETAILED DESCRIPTION OF THE INVENTION

Terms and symbols used herein have meanings consistent with usage in contemporarychemical literature unless otherwise noted.

For example, the term “(C₁₋₆) alkyl” includes saturated alkyl groups that may be branched or unbranched such as methyl, ethyl, n-propyl, isopropyl, n-butyl, isobutyl, sec-butyl, tert-butyl, pentyl, 2-pentyl, 3-pentyl, neopentyl, n-hexyl and the like.

The term “(C₁₋₄) alkyl” includes saturated and that may be branched or unbranched such as methyl, ethyl, n-propyl, isopropyl, n-butyl, isobutyl, sec-butyl, tert-butyl and the like.

The term “(C₁₋₄) alkyene” refers to straight chain alkylene groups such as —CH₂—, —CH₂CH₂—, —CH₂CH₂CH₂—, —CH₂CH₂CH₂CH₂—, or branched alylene groups such as —CH₂C(CH₃)₂—, or —H₂CH(CH₃)—, —H₂CH₂CH(CH₃)—, —CH₂CH(CH₃)CH₂—, and the like.

The term “(C₂₋₆) alkenyl” includes unsaturated alkyl groups that may be branched or unbranched having from two to six carbon atoms such as vinyl, allyl, 1-buten4-yl, 2-buten-4-yl, HC(═CH₂)CH₃, —CH═CH₂CH₂CH₃, —CH═C(CH₃)₂, —CH═CH—CH₂CH₂CH₃, —CH═CHCH₂CH₂CH₂CH₃ and the like.

The term “(C₃₋₆) cycloalkyl” refers to cyclopropyl, cyclobutyl, cyclopentyl, and cyclohexyl.

The term “halogen” includes fluoro, chloro, bromo and iodo.

The term “(C₁₋₆) fluoroalkyl” refers to a (C₁₋₆) alkyl group which is substituted with one to six fluorines, such as, fluoromethyl, difluoromethyl, trifluoromethyl, 2-fluoroethyl, 2,2,2-trifluoroethyl, 1,1,2,2,2-pentafluoroethyl, 3-fluoropropyl, 3,3,3-trifluoropropyl, 1,1,1,3,3,3-hexafluoroprop-2-yl, and 6-fluorohexyl and the like.

The term “(C₁₋₆) alkoxy” includes such groups as methoxy, ethoxy, isopropoxy, sec-butoxy, tert-butoxy, 2-pentoxy, 3-hexyloxy, and the like.

The term “(C₁₋₆) fluoroalkoxy” refers to a (C₁₋₆) fluoroalkyl group which is attached to an oxygen.

The term “(C₁₋₆) alkylthio” includes such groups as methylthio, ethylthio, isopropylthio, sec-butylthio, tert-butylthio, 1-hexylthio, and the like.

The term “acyl” includes, for example, formyl, acetyl, propanoyl, butanoyl, 2-methylpropanoyl, hexanoyl, and the like.

The term “(C₁₋₄)alkylsulfonyl” includes methanesulfonyl, ethanesulfonyl, propanesulfonyl, isopropanesulfonyl, 1-butanesulfonyl and the like.

The term “monocyclic heteroaromatic” refers to a five or six membered aromatic ring containing one to three heteroatoms selected from N, O, and S. Recognize that if one of the heteroatoms is O or S, the heteroaromatic ring must be a five membered ring and that any other heteroatoms contained therein must be N. “Monocyclic heteroaromatic” may be unsubstituted or substituted with one to three substituents inependently selected, from hydrogen, halogen, (C₁₋₆) alkyl, (C₁₋₆) fluoroalkyl, —OH, (C₁₋₆) alkoxy, (C₁₋₆) fluoroalkoxy, (C₁₋₆) thioalkyl, acyl, (C₁₋₄)alkylsulfonyl, —NO₂, —CN, carboxamido which may be substituted on the nitrogen by one or two (C₁₋₄) alkyl groups, and NH₂ in which one of the hydrogens may be replaced by a (C₁₋₄) alkyl group and the other hydrogen may be replaced by either a (C₁₋₄) alkyl group, an acyl group, or a (C₁₋₄) alkylsulfonyl group. Examples of such monocyclic heteroaromatic systems include furan, thiophene, pyridine, pyrimidine, thiazole, 1,2,3-triazole, and the like.

The term “bicyclic heteroaromatic” refers to a bicyclic aromatic system containing one to three heteroatoms selected from N, O, and S. Examples include indole, benzofuran, benzothiophene, quinoline, isoquinoline, indazole, benzothiazole, and the like. “Bicyclic heteroaromatic” may be unsubstituted or substituted with one to three substituents independently selected from hydrogen, halogen, (C₁₋₆) alkyl, (C₁₋₆) fluoroalkyl, —OH, (C₁₋₆) alkoxy, (C₁₋₆) fluoroalkoxy, (C₁₋₆) thioalkyl, acyl, (C₁₋₄)alkylsulfonyl, —NO₂, —CN, carboxamido which may be substituted on the nitrogen by one or two (C₁₋₄) alkyl groups, and NH₂ in which one of the hydrogens may be replaced by a (C₁₋₄) alkyl group and the other hydrogen may be replaced by either a (C₁₋₄) alkyl group, an acyl group, or a (C₁₋₄) alkylsulfonyl group. Examples of such monocyclic heteroaromatic systems include furan, thiophene, pyridine, pyrimidine, thiazole, 1,2,3-triazole, and the like.

The term “phenyl” refers to phenyl which may be unsubstituted or substituted with one to three substituents independently selected from hydrogen, halogen, (C₁₋₆) alkyl, (C₁₋₆) fluoroalkyl, —OH, (C₁₋₆) alkoxy, C₁₋₆) fluoroalkoxy, (C₁₋₆) thioalkyl, acyl, (C₁₋₄)alkylsulfonyl, —NO₂, —CN, carboxamido which may be substituted on the nitrogen by one or two (C₁₋₄) alkyl groups, and NH₂ in which one of the hydrogens may be replaced by a (C₁₋₄) alkyl group and the other hydrogen may be replaced by either a (C₁₋₄) alkyl group, an acyl group, or a (C₁₋₄) alkylsulfonyl group.

The term “tetrazole” refers to a tetrazole which may be unsubstituted or substituted with a (C₁₋₄) alkyl group.

In the case of optionally benzo-fused five or six member aromatic ring having zero to three hetero atoms independently selected from N, O, and S, the two atoms of the aromatic ring which are fused to the adjoining seven member ring are constrained to both be carbon. If the aromatic ring contains two additional adjacent carbon atoms, a benzene ring may be fused to the aromatic ring at those two adjacent carbon atoms. Examples of optionally benzo-fused five or six member aromatic rings having zero to three hetero atoms independently selected from N, S, and O include benzene, pyridine, furan, pyrrole, thiophene, thiazole, oxazole, pyrazole, imidazole, 1,2,3-triazole, naphthylene, quinoline, isoquinoline, indole, benzofuran, benzothiophene, and the like.

As used herein, when Y is a bond, R³-Y-Alk- is R³-Alk-.

The compounds of the present invention may, depending upon their structure and manner of synthesis and isolation, exist as a pharmaceutically acceptable solvate. These solvates include water, methanol, and ethanol. Solvated forms of the compounds of the present invention represent a further embodiment of the present invention.

The compounds of the present invention may, depending upon their structure and manner of synthesis and isolation, exist as a pharmaceutically acceptable hydrates. Hydrated forms of the compounds of the present invention represent a further embodiment of the present invention.

The compounds of formula (I) can exist in optically isomeric forms, i.e., stereoisomeric forms. That is, these compounds have a least one chiral, i.e., asymmetric, center at the carbon atom of the piperazine ring to which “Alk” is attached. Such asymmetry gives raise to at least one pair of enantiomers. An equal mixture of enantiomers is known as a “racemic” mixture or a “racemate.” The representation of formula (I) is intended to represent each of those stereoisomers and mixtures thereof.

The terms “R” and “S” are used herein as commonly used in organic chemistry to denote specific configuration of a chiral center. It is understood that compounds of the present invention may exist as stereoisomers. As such, all enantiomers, diastereomers, and mixtures thereof, are included within the scope of the present invention. Where specific stereochemistries are identified in this application, the Cahn-Prelog-Ingold designations of (R)- and (S)- and the cis and trans designation of relative stereochemistry are used to refer to specific isomers and relative stereochemistry. Some of the compounds of formula (I) may have two or more chiral centers.

Some of the compounds of the present invention may also be isomeric with respect to one or more double bonds, which introduces geometric, i.e., cis and trans, isomers. A discussion of optical and geometric isomers can be found in standard organic chemistry text books such as March 's Advanced Organic Chemistry, 5^{th} Ed., Chapter 4, Wiley-Interscience, John Wiley & Sons, Inc., New York (2001), hereinafter, “March”. Herein, when a compound of the present invention is named, or its structure presented, without an indication of asymmetric form, all of the possible asymmetric forms are intended. This invention is not limited to any particular isomer but includes all possible individual isomers and racemates.

Preferred among the compounds of formula (I) are those wherein:

a) The aromatic ring A is selected from the group consisting of:

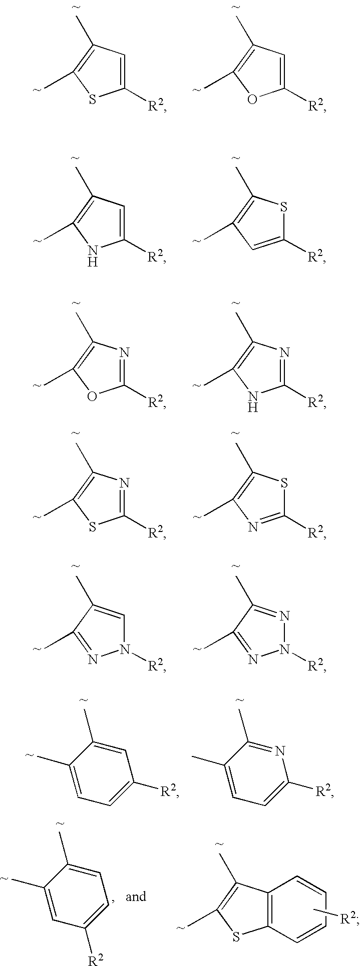

b) R¹ is hydrogen or (C₁₋₆) alkyl, wherein (C₁₋₆) alkyl is unsubstituted or substituted with OH, methoxy, ethoxy, —CN, imidazolidin-2-one, —CH₂CH₂OH or tetrazole wherein tetrazole is unsubstitued or substituted with (C₁₋₄)alkyl;

c) R² is H or (C₁₋₆) alkyl;

d) R³ is hydrogen, (C₂₋₆) alkenyl, phenyl, (C₁₋₆)fluoroalkyl, (C₁₋₄) alkyl wherein (C₁₋₄) alkyl is unsubstituted or substituted with a phenyl;

e) X is CH₂, S, or O;

f) Alk is (C₁₋₄) alkylene;

g) Y is O or a bond; and

h) The stereo configuration is “S” about the carbon of the piperazine group bound to Alk.

The compounds of formula (Ic), the compounds of formula (Id) the compounds of formula (Ie) listed in Table 1, Table 2, and Table 3 are of particular interest:

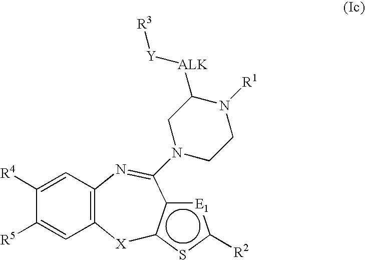

wherein R⁴ and R⁵ are hydrogen

TABLE 1

| Ex | | | | | | | |
|---|---|---|---|---|---|---|---|
| No: | X | E₁ | Alk | Y | R³ | R¹ | R² |
| 125 | CH₂ | CH | CH₂ | bond | Ph | H | CH₃ |
| 127 | CH₂ | CH | CH₂ | bond | Ph | CH₃ | CH₃ |
| 128 | CH₂ | CH | CH₂CH₂ | bond | Ph | H | CH₃ |
| 129 | CH₂ | CH | CH₂CH₂ | bond | Ph | CH₃ | CH₃ |
| 130 | CH₂ | CH | CH₂CH₂ | O | H | H | CH₃ |
| 131 | CH₂ | CH | CH₂CH₂ | bond | Ph | CH₂CH₂—O— | CH₃ |
| | | | | | | CH₂CH₂OH | |
| 132 | CH₂ | CH | CH₂CH₂ | bond | Ph | CH₂CH₂ | CH₃ |
| | | | | | | Imidazolidin-2- | |
| | | | | | | one | |
| 133 | CH₂ | CH | CH₂CH₂ | bond | Ph | CH₂CH₂— | CH₃ |
| | | | | | | CH₂CH₂CN | |
| 134 | CH₂ | CH | CH₂CH₂ | bond | Ph | CH₂CH₂— | CH₃ |
| | | | | | | CH₂CH₂-1- | |
| | | | | | | methyl-1H- | |
| | | | | | | tetrazol5-yl | |
| 135 | CH₂ | CH | CH₂CH₂ | bond | (4-CF₃)Ph | H | CH₃ |
| 136 | CH₂ | CH | CH₂CH₂ | bond | (4-CF₃)Ph | CH₃ | CH₃ |
| 146 | CH₂ | CH | CH₂CH₂ | bond | Ph | H | CH(CH₃)₂ |
| 147 | CH₂ | CH | CH₂CH₂ | bond | Ph | CH₃ | CH(CH₃)₂ |
| 148 | CH₂ | CH | CH₂ | bond | Ph | H | CH(CH₃)₂ |
| 149 | CH₂ | CH | CH₂ | bond | Ph | CH₃ | CH(CH₃)₂ |
| 150 | CH₂ | CH | CH₂ | bond | Ph | CH₂CH₂O— | CH(CH₃)₂ |
| | | | | | | CH₂CH₂OH | |
| 151 | CH₂ | CH | CH₂CH₂ | bond | Ph | CH₂CH₂O— | CH(CH₃)₂ |
| | | | | | | CH₂CH₂OH | |
| 152 | CH₂ | CH | CH₂CH₂ | bond | Ph | CH₂CH₂O— | CH(CH₃)₂ |
| 153 | CH₂ | CH | CH₂CH₂ | bond | Ph | CH₂CH₂O— | CH(CH₃)₂ |
| 154 | CH₂ | CH | CH₂CH₂ | bond | (4-OCH₃)Ph | CH₃ | CH(CH₃)₂ |
| 166 | C═O | CH | CH₂CH₂ | bond | Ph | H | CH₃ |
| 167 | C═O | CH | CH₂CH₂ | bond | Ph | CH₃ | CH₃ |
| 168 | C═O | CH | CH₂ | bond | Ph | H | CH₃ |
| 169 | C═O | CH | CH₂ | bond | Ph | CH₃ | CH₃ |
| 170 | C═O | CH | CH₂ | bond | Ph | CH₂CH₂—O— | CH₃ |
| | | | | | | CH₂CH₂OH | |
| 172 | CH₂ | N | CH₂CH₂ | O | CH₃ | H | CH₃ |
| 173 | CH₂ | N | CH₂CH₂ | O | CH₃ | CH₃ | CH₃ |
| 174 | CH₂ | N | CH₂CH₂ | bond | (4-OCH₃)Ph | H | CH₃ |
| 175 | CH₂ | N | CH₂CH₂ | bond | (4-OCH₃)Ph | CH₃ | CH₃ |
| 176 | CH₂ | N | CH₂CH₂ | bond | (3-F)Ph | H | CH₃ |
| 177 | CH₂ | N | CH₂CH₂ | bond | (4-F)Ph | H | CH₃ |
| 178 | CH₂ | N | CH₂CH₂ | bond | (4-F)Ph | CH₃ | CH₃ |
| 179 | CH₂ | N | CH₂CH₂ | bond | (3-F)Ph | CH₃ | CH₃ |
| 180 | CH₂ | N | CH₂CH₂ | bond | (4-OCH₃)Ph | H | CH(CH₃)₂ |
| 181 | CH₂ | N | CH₂CH₂ | bond | (4-F)Ph | H | CH(CH₃)₂ |
| 182 | CH₂ | N | CH₂CH₂ | O | CH₃ | H | CH(CH₃)₂ |
| 183 | CH₂ | N | CH₂CH₂ | bond | (4-OCH₃)Ph | H | CH₂C(CH₃)₂ |
| 184 | CH₂ | N | CH₂CH₂ | bond | (4-F)Ph | H | CH₂C(CH₃)₂ |
| 185 | CH₂ | N | CH₂CH₂ | bond | (4-OCH₃)Ph | H | C(CH₃)4 |
| 187 | CH₂ | N | CH₂CH₂ | O | CH₃ | H | C(CH₃)4 |
| 188 | CH₂ | N | CH₂CH₂ | bond | (3-F)Ph | H | C(CH₃)4 |
| 189 | CH₂ | N | CH₂CH₂ | bond | (4-F)Ph | H | C(CH₃)4 |
| 190 | CH₂ | N | CH₂CH₂ | bond | (4-OCH₃)Ph | CH₃ | CH(CH₃)₂ |
| 191 | CH₂ | N | CH₂CH₂ | bond | (4-F)Ph | CH₃ | CH(CH₃)₂ |
| 193 | CH₂ | N | CH₂CH₂ | O | CH₃ | CH₃ | CH(CH₃)₂ |
| 194 | CH₂ | N | CH₂CH₂ | bond | (4-OCH₃)Ph | CH₃ | CH₂CH(CH₃)₂ |
| 195 | CH₂ | N | CH₂CH₂ | bond | (4-F)Ph | CH₃ | CH₂CH(CH₃)₂ |
| 196 | CH₂ | N | CH₂CH₂ | bond | (4-OCH₃)Ph | CH₃ | C(CH₃)3 |
| 198 | CH₂ | N | CH₂CH₂ | O | CH₃ | CH₃ | C(CH₃)3 |
| 199 | CH₂ | N | CH₂CH₂ | bond | (4-F)Ph | CH₃ | C(CH₃)3 |
| 200 | CH₂ | N | CH₂CH₂ | bond | (4-OCH₃)Ph | CH₃ | CH(CH₃)₂ |
| 201 | CH₂ | N | CH₂CH₂ | bond | (3-F)Ph | CH₃ | C(CH₃)3 |
| 220 | S | CH | CH₂CH₂ | O | CH₃ | H | CH₃ |
| 222 | S | CH | CH₂CH₂ | bond | Ph | H | CH₃ |
| 225 | S | CH | CH₂CH₂ | O | CH₃ | H | CH₂CH₃ |
| 227 | S | CH | CH₂CH₂ | O | CH₃ | H | CH(CH₃)₂ |
| 228 | S | CH | CH₂CH₂ | bond | (3-F)Ph | H | CH(CH₃)₂ |
| 229 | S | CH | CH₂CH₂ | bond | (4-OCH₃)Ph | H | CH(CH₃)₂ |
| 230 | S | CH | CH₂CH₂ | bond | (4-OCH₃)Ph | CH₃ | CH(CH₃)₂ |
| 231 | S | CH | CH₂CH₂ | O | CH₃ | CH₃ | CH₂CH₃ |
| 232 | S | CH | CH₂CH₂ | O | CH₃ | CH₃ | CH(CH₃)₂ |
| 233 | S | CH | CH₂CH₂ | bond | (3-F)Ph | CH₃ | CH(CH₃)₂ |
| 234 | S | CH | CH₂CH₂ | O | CH₃ | CH₃ | CH₃ |
| 235 | S | CH | CH₂CH₂ | bond | Ph | CH₃ | CH₃ |
| 237 | S | CH | CH₂CH₂ | bond | (3-F)Ph | H | CH₂CH(CH₃)₂ |
| 238 | S | CH | CH₂CH₂ | bond | (3-F)Ph | CH₃ | CH₂CH(CH₃)₂ |
| 240 | S | CH | CH₂CH₂ | O | CH₃ | H | CH₂CH₂CH₃ |
| 241 | S | CH | CH₂CH₂ | O | CH₃ | CH₃ | CH₂CH₂CH₃ |
| 242 | S | CH | CH₂CH₂ | bond | (4-OCH₃)Ph | H | CH₂CH₂CH₃ |
| 243 | S | CH | CH₂CH₂ | bond | (4OCH₃)Ph | CH₃ | CH₂CH₂CH₃ |
| 244 | S | CH | CH₂CH₂ | bond | (3-F)Ph | H | CH₂CH₂CH₃ |
| 245 | S | CH | CH₂CH₂ | bond | (3-F)Ph | CH₃ | CH₂CH₂CH₃ |
| 336 | S | N | CH₂CH₂ | bond | (3-F)Ph | H | H |
| 337 | S | N | CH₂CH₂ | bond | (4-OCH₃)Ph | H | CH₂CH₃ |
| 338 | S | N | CH₂CH₂ | bond | (4-OCH₃)Ph | H | CH(CH₃)₂ |
| 339 | S | N | CH₂CH₂ | bond | (3-F)Ph | H | CH₂CH₃ |
| 340 | S | N | CH₂CH₂ | bond | (3-F)Ph | H | CH(CH₃)₂ |
| 341 | S | N | CH₂CH₂ | O | CH₃ | H | |
| 342 | S | N | CH₂CH₂ | bond | (4-OCH₃)Ph | CH₃ | CH₂CH₃ |
| 343 | S | N | CH₂CH₂ | bond | (3-F)Ph | CH₃ | CH₂CH₃ |
| 344 | S | N | CH₂CH₂ | bond | (4-OCH₃)Ph | CH₃ | CH(CH₃)₂ |
| 345 | S | N | CH₂CH₂ | bond | (3-F)Ph | CH₃ | H |
| 346 | S | N | CH₂CH₂ | O | CH₃ | CH₃ | H |
| 350 | S | N | CH₂CH₂ | bond | (3-F)Ph | CH₃ | C(O)CH₃ |
| 351 | S | N | CH₂CH₂ | O | CH₃ | CH₃ | C(O)CH₃ |

all salts, solvates, optical and geometric isomers, and crystalline forms thereof. Ex.
No. corresponds to example number in the Examples section.

| | |
|---|---|
| | (Id) |
| 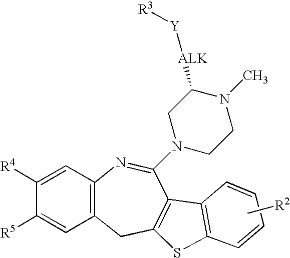 | |

wherein R¹ is methyl, R² is hydrogen

TABLE 2

| Ex. No.: | Alk | Y | R³ | R4 | R5 |
|---|---|---|---|---|---|
| 156 | CH₂CH₂ | O | CH₃ | H | H |
| 159 | CH₂CH₂ | O | CH₃ | H | Cl |
| 160 | CH₂CH₂ | O | CH₃ | H | F |
| 161 | CH₂CH₂ | O | CH₃ | F | F |

all salts, solvates, optical and geometric isomers, and crystalline forms thereof.
Ex. No. corresponds to example number in the Examples section.

| | |
|---|---|
| | (Ie) |
| 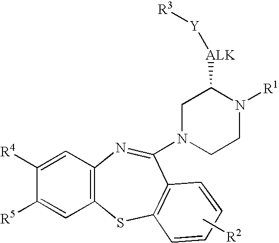 | |

R², R⁴ and R⁵ are hydrogen

TABLE 3

| Ex No: | Alk | Y | R³ | R¹ |
|---|---|---|---|---|
| 255 | CH₂CH₂ | bond | Ph | CH₃ |
| 256 | (R)CH₂ | bond | Ph | H |
| 257 | (R)CH₂ | bond | Ph | CH₃ |
| 258 | CH₂CH₂ | bond | Ph | H |
| 259 | CH₂ | bond | Ph | H |
| 260 | (S)CH₂ | bond | Ph | CH₃ |

all salts, solvates, optical and geometric isomers, and crystalline forms thereof.
Ex. No.: corresponds to example number in the Examples section.

Since the compounds of this invention are basic in nature, they react with any of a number of inorganic and organic acids to form acid addition salts. For the therapeutic utility taught herein, the salt of the claimed compounds must be pharmaceutically acceptable. Acids commonly employed to form pharmaceutically acceptable salts are inorganic acids such as hydrochloric acid, hydrobromic acid, hydroiodic acid, sulfuric acid, phosphoric acid, and organic acids, such as p-toluenesulfonic. acid, methanesulfonic acid, oxalic acid, p-bromo-phenylsulfonic acid, carbonic acid, succinic acid, citric acid, benzoic acid, acetic acid, lactic acid, malaic acid, tartaric acid, and the like. For further details on pharmaceutically acceptable salts, see Journal of Pharmaceutical Science, 66, 1 (1977). Salts that are not pharmaceutically acceptable may be used as intermediates to prepare other compounds of formula (I) or a pharmaceutically acceptable salt of compounds of formula (I) and are within the scope of the present invention. Particular pharmaceutically acceptable salts are those formed with hydrochloric acid sulfuric, or phosphoric acid.

The intermediates and final products described herein may be isolated and purified by the conventional techniques known to artisans of organic chemistry. For example, the well-known techniques of chromatography, recrystallization, distillation, and sublimation may be used singularly and sequentially.

General Synthetic Methods

Compounds of formula (I) of this invention can be prepared by several methods generally known in the art of organic chemistry. Starting materials, the preparation of which are not described, are commercially available or can be readily prepared by known techniques from commercially available starting materials.

Compounds of formula (Ia) in which R¹ is hydrogen can be converted to compounds of formula (Ib) in which R¹ is defined above but does not equal hydrogen. This transformation can be accomplished, as shown in Scheme 1 by treatment of formula (Ia) with an alkylating agent. Alkylating agents include alkyl halides and alkyl sulfonate esters. Examples include but are not limited to, methyl iodide, 1-bromobutane, 2-propyl methanesulfonate, and bromoethylmethyl ether. This reaction is usually performed in the presence of a base and solvent. The base can be either an organic base such as pyridine or diisopropylethylamine or an inorganic base such as potassium carbonate. Solvents include methanol, ethanol, THF, and DMF. This transformation can also be accomplished by reductive alkylation of the piperazine by treatment with an aldehyde or ketone under reducing conditions. Examples of suitable aldehydes include formaldehyde, acetaldehyde, propionaldehyde, butyraldehyde, isobutyraldehyde, and the like. Suitable ketones include acetone, methylethylketone, and the like. Reductive alkylations are often performed under catalytic hydrogenation conditions. Other reducing agents include formic acid, sodium borohydride, sodium cyanoborohydride, and sodium triacetoxyborohydride. This transformation can also be accomplished by acylation of the piperazine nitrogen to form an amide and reduction of the amide to yield the alkylated piperazine. Examples of acylating agents include acyl halides such as acetyl chloride, propionyl chloride, pivaloyl chloride, and cyclopropylcarbonyl chloride, carboxylic acid anhydrides such as formylacetic anhydride and acetic anhydride, and carboxylic acids in the presence of an activating agent such as dicyclohexylcarbodiimide or carbonyldiimidazole. The resulting amides may be reduced to the tertiary amines with reducing agents such as lithium aluminum hydride or borane.

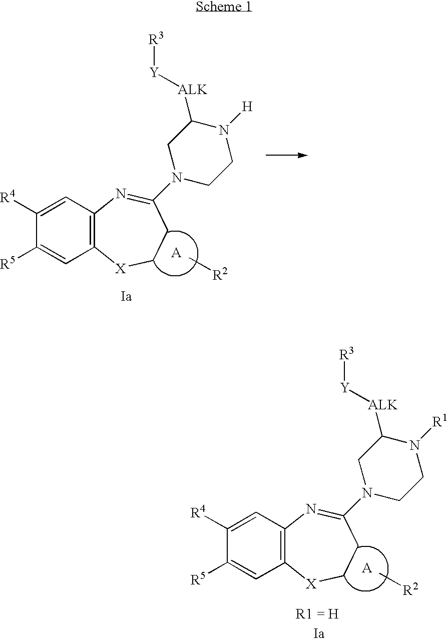

As shown in Scheme 2, compounds of formula (I) may be prepared by reacting an appropriately substituted piperazine of formula (V) with a tricyclic intermediate of im formula (IV). “LG” represents a leaving group examples of which include NH₂, halo, OY₁, or SY₁, wherein Y₁, is lower alkyl such as methyl, ethyl, or propyl or optionally substituted phenyl or OP(═O)R¹². R¹² can be morpholine. This reaction may conveniently be performed with heating in a solvent such as DMSO, toluene, IPA, DMF, and N-methylpyrrolidinone or a mixture of solvents such as DMSO and toluene in ratios of (1:2, 1:3, or 1:4). For compounds of formula (II) when LG is SY₁, the equivalence of piperazine maybe reduced to 1 to 2 when heating in IPA. 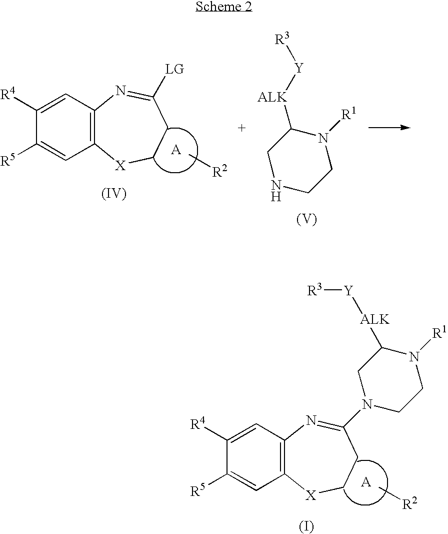
Alternatively, as shown in Scheme 3. tricyclic amide and thioamide intermediates of formula (VI) wherein Z is O or S, respectively, can react with substituted piperazines of formula (V) to give corresponding compounds of formula (I). This reaction is conventiently performed in a polar solvent and may be performed in the presence or absence of a Lewis acid such as TiCl₄.

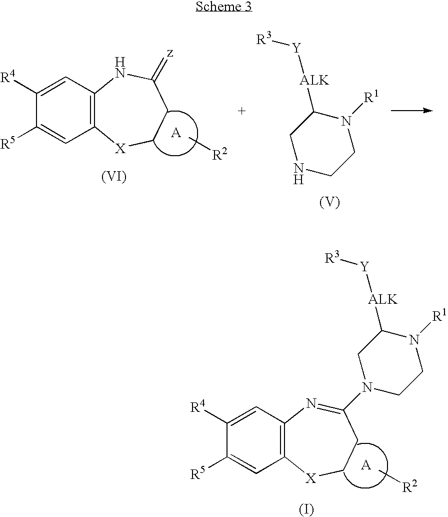

In Scheme 4 compounds of formula (VIb), wherein Z is S, may be prepared from compounds of formula (VIa), wherein Z is O, by treatment with a debydrative thiolating agent in the presence of an inert solvent. Examples of such dehydrative thiolating agents include P₂S₅ and Lawesson's reagent (2,4-bis(4-methoxyphenyl)-1,3-dithia-2,4-diphosphetane-2,4-disulfide). For a description of Lawesson's reagent and its use, see M. P. Cava and M. I. Levinson, Tetrahedron, 41, 5061 (1985).

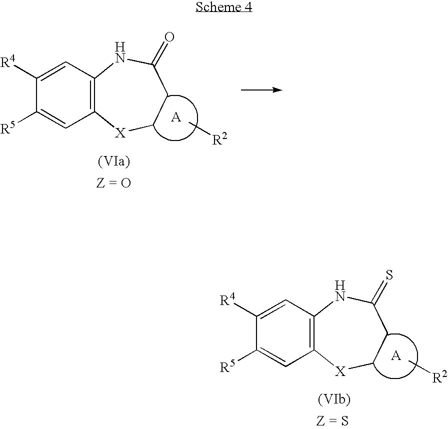

As shown in Scheme 5. tricyclic intermediates of formula (IV) can be prepared from the correspondhng ticyclic amide and thioamide intermediates of formula (VI). O-alkylation of an amide of formula (VI) (Z=O) provides an iminoether of formula (IV) (LG=OY₁). Suitable allating agents include Meerwein's reagent and methyl fluorosulfonate. Iminothioethers of formula (IV), wherein LG is SY₁, may be prepared by S-alkylation of thioamides of formula (VI), wherein Z is S. Suitable alkylating agents include allyl halides, alkyl sulfonates such as methyl trifluoromethanesulfonate, Meerwein's reagent and methyl fluorosulfonate. Reaction of an amide of formula (VI), wherein Z is O, with a dehydrative halogenating agent provides an iminohalide of formula (IV), wherein LG is a halo group. Suitable dehydrative halogenating agents include POCl₃, SOCl₂, PCl₃, PCl₅, PBr₃, PPh₃/Br₂, P(OPh)₃/I₂ and PPh₃/MeI.

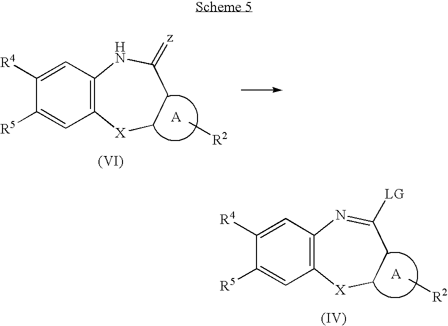

Compounds of formula (IV) in which LG is NH₂, OY₁ or SY₁ may be prepared from compounds of formula (IV), wherein LG is halo, by reaction with a suitable nucleophile, such as ammonia, an alcohol, or a thiol to give compounds of formula (IV), wherein LG is NH₂, OY₁ or SY₁, respectively. This reaction may be conveniently performed in a solvent and under basic conditions such as K₂CO₃.

As shown in Scheme 6, compounds of formula (I) may also be prepared by ring closure of an intermediate of formula (XIII a). This reaction may be effected by treatmhent of an amide of formula (XIII a) with an activating agent in the presence of an inert solvent. Examples of such activating agents include TiCl₄, POCl₃, P₂S₅, and Lawesson's reagent.

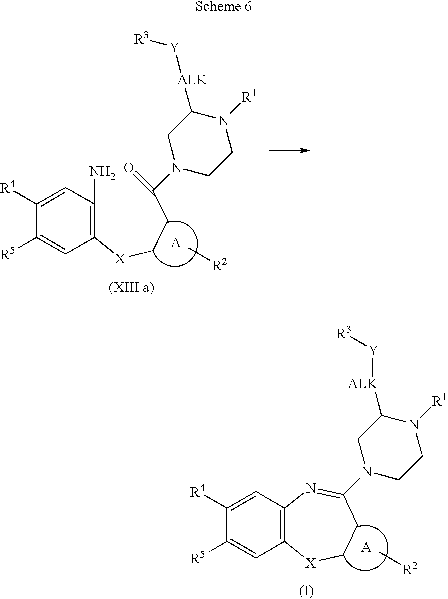

According to Scheme 7, compounds of formula (VI a), may be prepared by cyclization of an amine compounds of formula (XIII b) in which Y₂ is OY₇ or NY₈Y₉ wherein Y₇, Y₈ and Y₉ are independently, hydrogen or lower alkyl such as methyl, ethyl, or propyl.

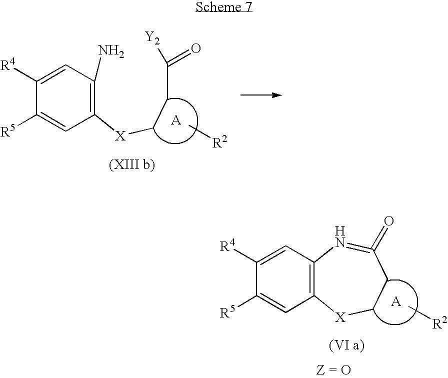

As seen in Scheme 8. amines of formula (XIII b) may be prepared from compounds of formula (XIII c). The symbol Y₃ represents a group that may be converted to an amino group, such as NO₂, COOH, and NHCOOY₄, wherein Y₄ may be an optionally substituted alkyl such as, but not limited to, methyl, ethyl, 2-phenylethyl, t-butyl, 2-(trimethylsilyl)ethyl, 2,2,2-trichloroethyl, vinyl, allyl or optionally substituted benzyl group such as, but not limited to, benzyl, p-methoxybenzyl, p-nitrobenzyl, or diphenylmethyl

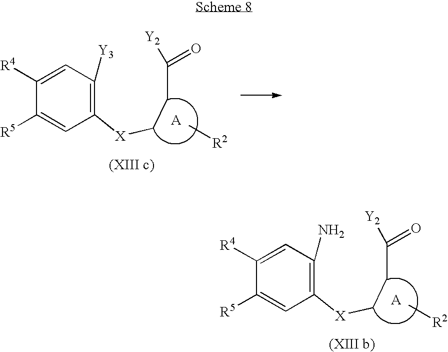

If Y₃ is NO₂, treatment of compounds of formula (XIII c) under reducing conditions will provide corresponding compounds of formula (XIII b). Examples of such reducing conditions include catalytic hydrogenation conditions or SnCl₂. Compounds of formula (XIII C), wherein Y₃ is NHCOOY₄, may be converted to the corresponding compounds of formula (XIII b) under conditions that allow for removal of the COOY₄ group. If Y₄ is optionally substituted alkyl, such conditions may include hydrolysis under acidic or basic conditions. If Y₄ is optionally substituted benzyl, treatment under reducing conditions, preferably catalytic hydrogenation conditions, provides the corresponding compound of formula (XIII b). If Y₄ is t-butyl, treatment with acid provides a compound of formula (XIII b). If Y₄ is 2,2,2-trichloroethyl, reducing conditions, preferably zinc metal in acidic medium, yield a compound of formula (XIII b). If Y₄ is 2-(trimethylsilyl)ethyl, treatment with fluoride ion yield a compound of formula (XIII b).

Compounds of formula (XIII b) may also be prepared by Curtius rearrangement of the correspondent compound of formula (XIII c) in which Y₃ is COOH. The Curtius rearrangement occurs by thermal rearrangement of the acylazide of formula (XIII c) in which Y₃ is CON₃ to yield the isocyanate of formula (XIII C) in which Y₃ is NCO. This isocyanate may be hydrolyzed either directly or through the urethane in which Y₃ is NHCO₂Y₄, to yield the corresponding compound of formula (XIII b).

According to Scheme 9, compounds of formula (IVa) in which LG is NH₂ may be prepared by cyclization of aminonitrile compounds of formula (XIIId). 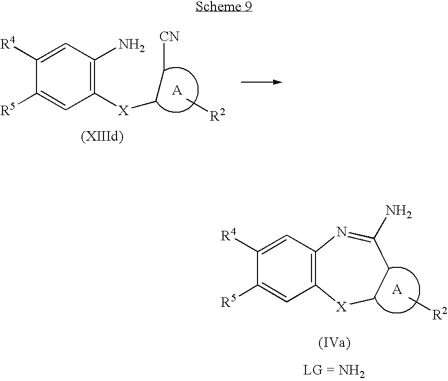
According to Scheme 10, aminonitrile compounds of formula (XIII d) may be prepared from corresponding compounds of formula (XIII e), in the manner described for Scheme 8. Alternatively, compounds of formula (XIII d) may be prepared by Curtius rearrangement under conditions also described for Scheme 8.

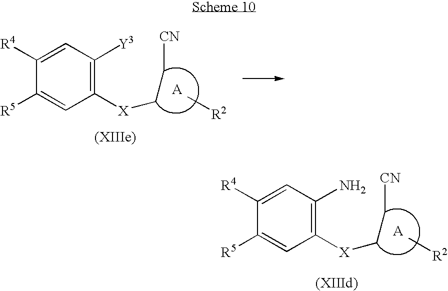

As shown in Scheme 11, compounds of formula (XIII a), wherein all groups are defined as above, may be prepared from corresponding compounds of formula (XIII f) in which Y₃ is a group that may be converted to an amino group.

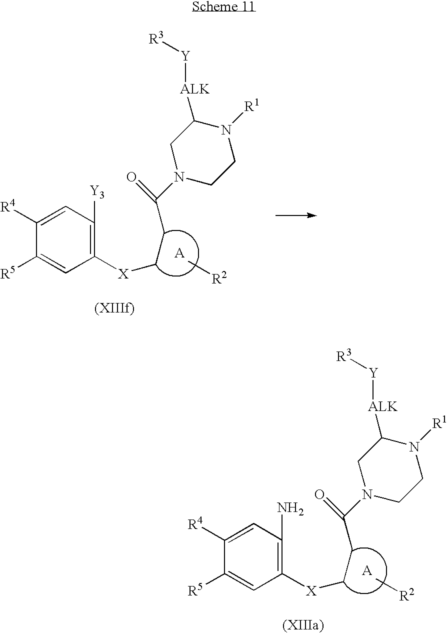

According to Scheme 12, compounds of formula (XIII f), wherein Y₃ is a group that may be converted to an amino group as defined above, and all other groups are as defined above, may be prepared by coupling a compound of formula (V) with a compound of formula (XIII g). Such coupling reactions may be performed under conditions commonly employed to form amide bonds. Coupling reagents include dicyclohexylcarbodnmide (DCC), diphenylphosphorylazide (DPPA), and 1-(3-dimethylaminopropyl)-3-ethylcarbodiimide hydrochloride (EDC).

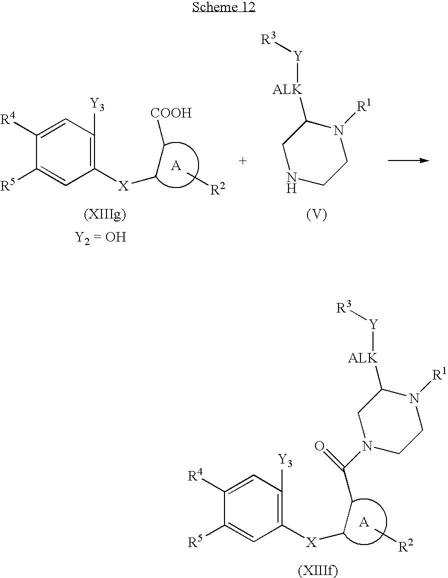

As shown in Scheme 13, compounds of formula (XIII) in which Y₃ may be NH₂ or a group that may be converted to an amino group as described above, Y₁₀ may be CN, COOY₇ or CONY₈Y₉, in which Y₇, Y₈, and Y₉ may independently be hydrogen or lower alkyl, or NY₈Y₉ is the group (XVI), and the other groups are defined as above, may be prepared by reaction of compounds of formula (XIV) in which Y₁₁ may be a halo group or OSO₂CF₃ with compounds of formula (XV). This reaction may be performed under basic conditions in a polar, aprotic solvent. Suitable bases include NaH, KH, potassium tert-butoxide, and cesium carbonate. Suitable solvents include DMF, N-methylpyrrolidinone, and THF. The coupling of compounds of formula (XIV) with compounds of formula (XV a) to yield a compound of formula (XIII) may also be performed in the presence of a metal catalyst. Conditions for this transformation may be found in Hartwig, Angew. Chem. Int. Ed. (199 )37, 2046-2067, Wolff, et al., Acc. Chem. Res. (1998), 31, 805-818, Yang and Buchwald, J. Organomet. Chem. (1999) 576, 125-146, and references cited therein.

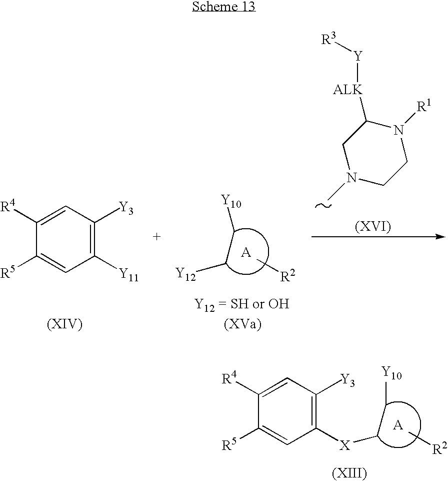

Compounds of formula (XIV) may be prepared by methods known in the art.

Alternatively as shown in Scheme 14, compounds of formula (XIII) in which Y₃ may be NH₂ or a group that may be converted to an amino group as described above, Y₁₀ may be CN, COOY₇ or CONY₈Y₉, in which Y₇, Y₈, and Y₉ may independently be hydrogen or lower alkyl; or NY₈Y₉ is the group (XVI), and the other groups are defined as above, may also be prepared by reaction of compounds of formula (XVI a) with compounds of formula (XV) in which Y₁₂ maybe a halo group or OSO₂CF₃. This reaction may be performed under basic conditions in a polar, aprotic solvent. Suitable bases include NaH, KH, potassium tert-butoxide, lithium hydroxide, and cesium carbonate. Suitable solvents include DMF, N-methylpyrrolidinone, and THF. The coupling of compounds of formula (XVI a) with compounds of formula (XV) to yield a compound of formula (XIII) may also be performed in the presence of a metal catalyst. Conditions for this transformation may be found in Hartwig, Angew. Chem. Int. Ed. 37, 2046-2067, (1998), Wolff, et al., Acc. Chem. Res., 31, 805-818, (1998), and Yang and Buchwald, J. Organomet. Chem. 576, 125-146, (1999), and references cited therein.

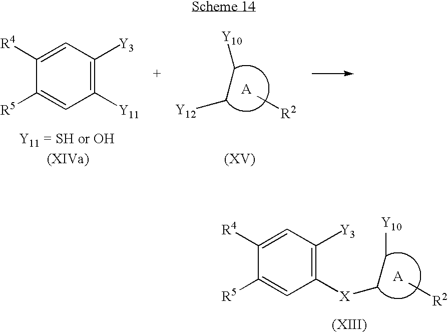

Compounds of formula (XVIa) may be prepared by methods known in the art.

According to Scheme 15, a compound of formula (VIa) can also be prepared by cyclization of isocyanate (XIIIh) under acidic conditions. Isocyanate (XIIIh) may be prepared from compounds of formula (XIII) in which Y₁₀ is hydrogen and Y₃ is an amino group by reaction with formic acetic anhydride and dehydration of the resulting formamide with a dehydrating agent such as POCl₃ or P₂O₅. Isocyanate (XIIIh) may also be prepared from compounds of formula (XIII) in which Y₁₀ is hydrogen and Y₃ is COOH by Curtius rearrangement as described before. Alternatively, a compound of formula (IIb) may also be prepared by reaction urea (XIIIi) in the presence of a Lewis acid. Urea (XIIIi) may be prepared by reaction of isocyanate (XIIIh) with an amine of formula (V).

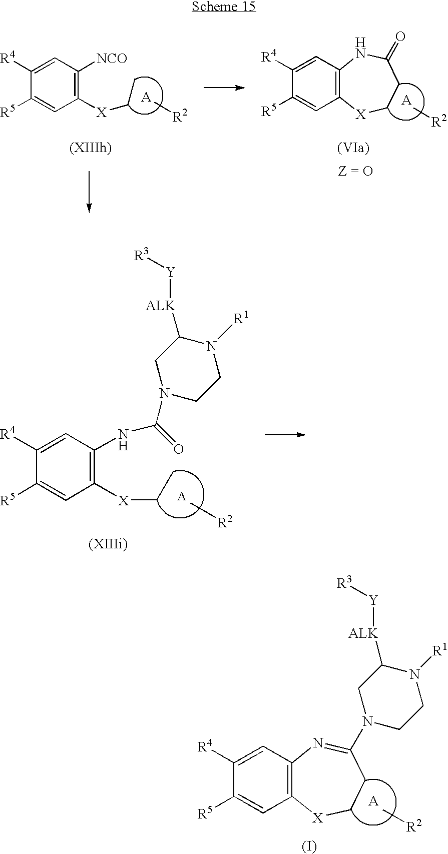

Methods for the preparation of compounds of formula (XV b) and formula (XV c) are known in the art and vary depending on the nature of the aromatic ring A,

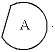

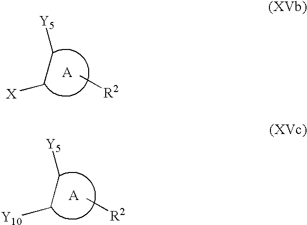

The skilled artisan will recognize that substituents R² and R⁴ and R⁵ in the compounds of foumula (I) may be present in the precursor molecules of formulas (XIV), (XIVa), (XVb), and (XVc). Alternatively, these substituents may be introduced at any convenient point during the synthesis either by replacement of a hydrogen (through, for example, an electrophilic aromatic substitution reaction) or by conversion of an existing substituent into the substituents present in the compounds of formula (I). Examples of electrophilic aromatic substitution reactions include halogenation, nitration, Friedel-Crafts acylation, and electrophilic trifluoromethylation under conditions described in the literature. Examples of conversion of an existing substituent into one present in the final compound include conversion of a Br substituent into a substituent such as SR¹¹ or COR¹¹ by metallation with an organolithium reagent and reaction with an electrophile such as R¹¹SSR¹¹ or R¹¹COOMe. R¹¹ may be (C₁₋₆)alkyl, (C₁₋₆) fluoroalkyl, benzyl, or optionally substituted phenyl” Additionally, a Br substituent can be converted to an optionally substituted aromatic ring by reaction with an optionally substituted phenylboronic acid in the presence of a palladium catalyst. Many other such finctional group transformations are reported in the literature.

More specifically, compounds of formula (VIc), wherein X is equal to CH₂, CO, COH(Me) and such, may be prepared as outlined in Scheme 16. For example, addition of a metallated aromatic or heteroaromaticspecies such as a lithiated thiophene, a Grignard reagent such as a thienylmagnesiumhalide or the like, compound of formula (XVd) to an amino cyano aromatic compound of formula (XIV b) in a solvent such as THF or ether like solvent at a low temperature at or about minus 78 degrees C., and allowing the reaction to warm to about room temperature, followed by treatment with an aqueous mineral acid, such as HCl gives a ketone, compound of formula (XIII j) which can then be deoxygenated by treatment with AlH₃ (formed in situ by the treatment of AlCl₃ with LAH) to give the methylene compound of formula (XIII k). The methylene compound may be treated with a reagent such as phosgene, to form an isocyanate which when treated with a reagent such as polyphosphoric acid (PPA) and heating above 100 degrees C., gives ring closure to form the tricycle compound of formula (VI c) where Z=O.

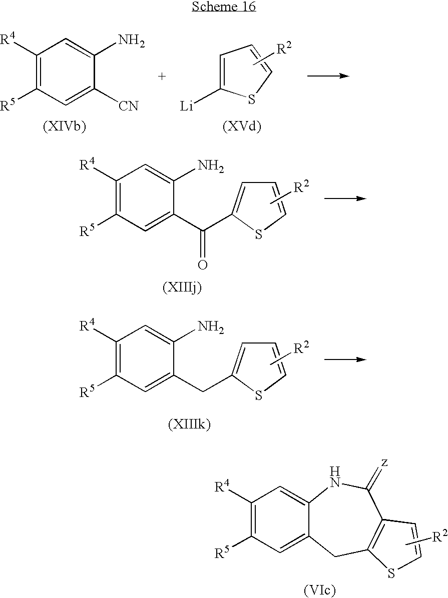

Additionally, compounds of formula (IVb) may be prepared as outlined in Scheme 17, whereby a metallated aromatic or heteroaromatic species compound of formula (XV e), of appropriate substitution, is allowed to add to a an aromatic nitro aldehyde, compound of formula (XIII l), at low temperature, in an ethereal solvent. The resulting alcohol is then deoxygenated either directly by treatment with a reducing agent such as InCl₃/SiH(CH₃)₂Cl or I₂/H₃PO₂ in an appropriate solvent, or oxidized to a ketone with such oxidants as pyridiniurn dichromate (PDC) in methylene chloride, and then reduced to the methylene compound of formula (XIII m) with Zn/acetic acid on heating. The compound is ring closed, by reduction of the nitro-functionality to amine by such as SnCl₂ in acid, which spontaneously cyclizes, on continued heating in acid to the cyclic amidine, compound of formula (IV b).

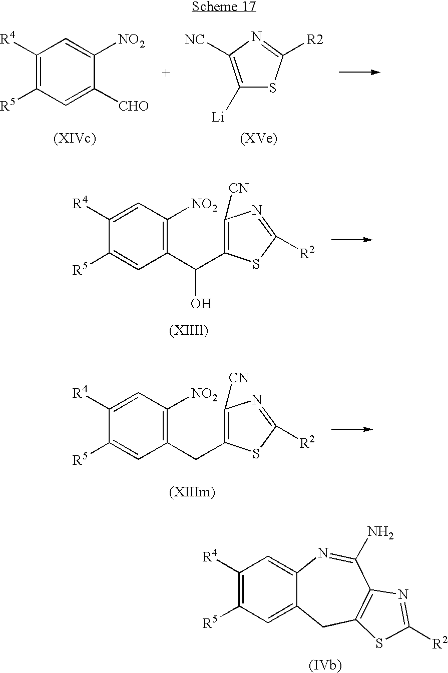

More specifically, the S-linked tricyclic systems containing a thiazole ring are described in Schemes 18, 19, 20 and 21.

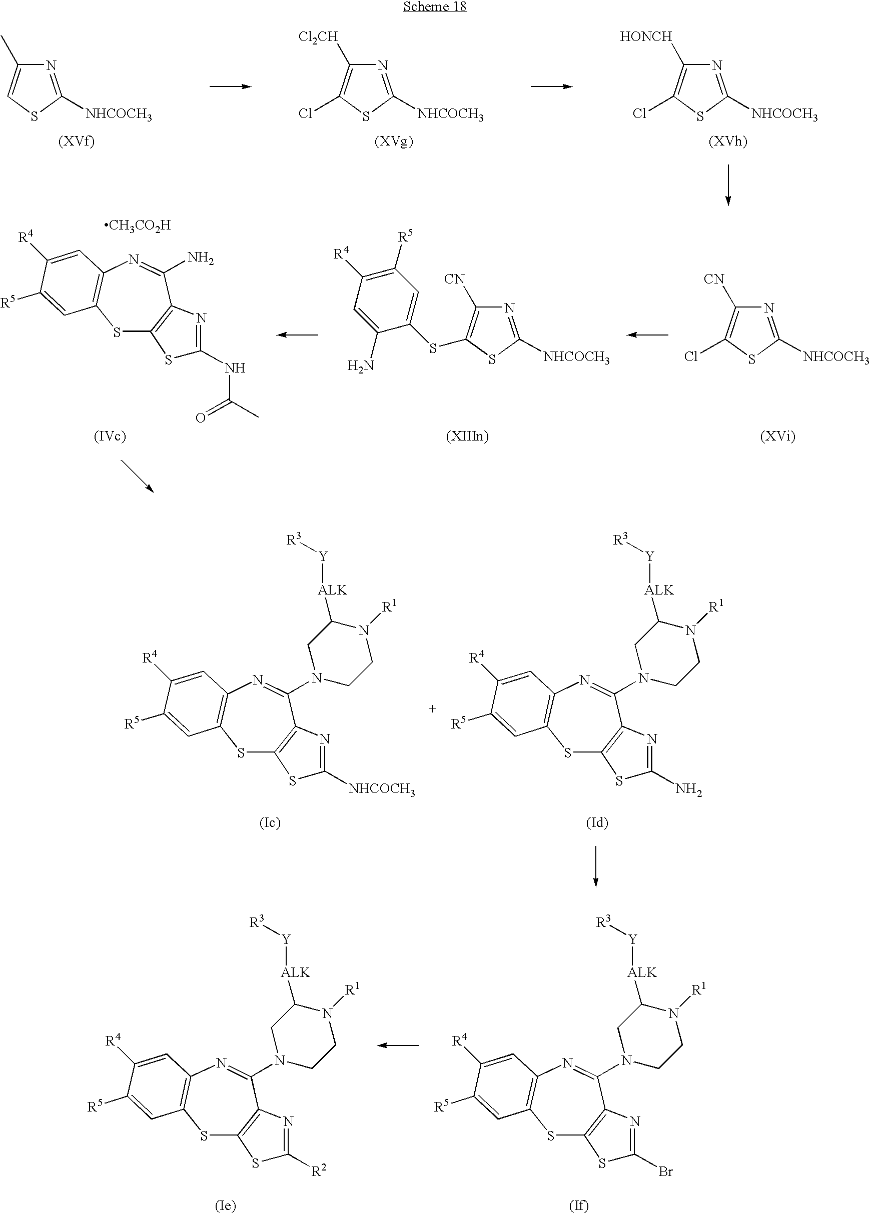

Piperazine containing tricyclic derivatives of structures (Ie) and (If) are formed by amidine exchange from compounds of formula (IVc) using, for example, compounds of formula (V) in a solvent mixture of, for example, toluene and methyl sulfoxide at elevated temperature. The tricyclic amidines of general formula (IVc) are formed by acid catalysed cyclisation of the intermediate aminonitriles of formula (XIIIn) using, for instance, acetic acid at elevated temperature to give the amidine as its acetete salt. The intermediate aminonitriles of formula (VIIIn) are made by base catalysed condensation of halonitrile intermediates of formula (XVi) with an ortho aminothiophenol derivative. Suitable bases are alkali metal carbonates such as, for instance, cesium carbonate in a polar aprotic solvent such as dinethylformamide. The required halonitrile derivatives (XVi) are made from commercially available 2-acetamido-4-methylthiazole by a three step procedure described in EP 0160818 which uses the analogous 4-methyl-2-phthalimidothiazole. The intermediate 2-aminothiazole tricyclic derivative (Id) can be further modified by diazotization and reaction of the intermediate diazonium salt with, for instance, cuprous bromide to give the 2-bromo derivative (If). Metal halogen exchange of the 2-bromine atom in (If) using, for instance, n-butyl lithium gives the 2-lithio species which can be quenched with electrophiles such as methyl iodide to give the 2-methyl derivative (Ie).

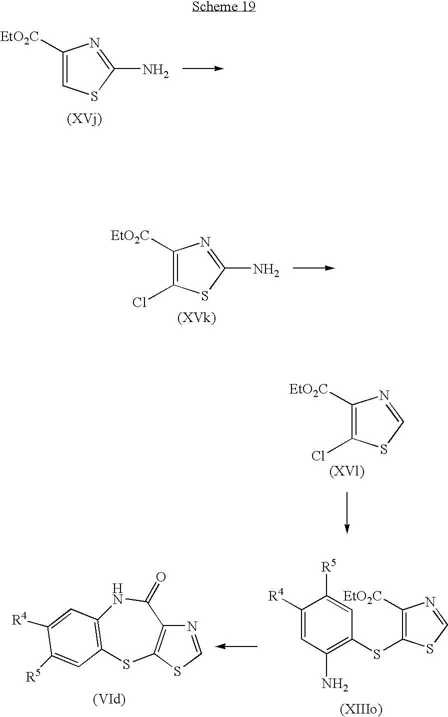

In Scheme 19, compounds of the formula (VId) can be prepared by acid catalysed ring closure of (XIIIo) in an inert solvent such as toluene. Suitable acid catalysts include para-toluene sulfonic acid, acetic acid or similar organic acids. The required intermediates for the generation of (XIIIo) can be prepared thus. Intermediate (XVj) by application of literature method as in Tetrahedron: Asymmetry 9 (1998) 1395-1407 and intermediate (XVk) by methods detailed in J. Heterocyclic Chem., 28 (1991) 1003-1011. A modified Doyle procedure can be used to prepare (XVl) by diazotization and quenching resulting diazonium salt with water. Examples of reagents which can be used to effect this conversion include t-butyl nitrite and isoamyl nitrite with dimethylformamide as the solvent. Reaction of the chloroester (XVl) with ortho amino thiophenol with a base in a suitable solvent under classic displacement conditions generates (XIV). Such bases include cesium carbonate and sodium hydride. Suitable solvents include dimethylformamide, tetrahydrofuran and other aprotic polar solvents.

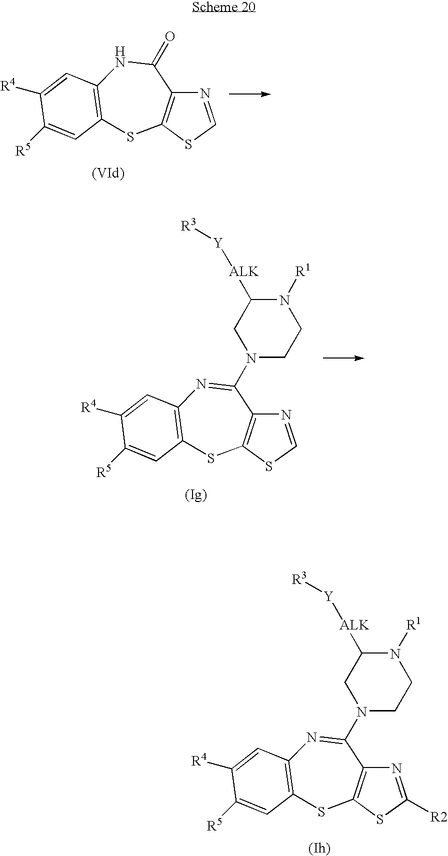

In Scheme 20, (Ig) can be further modified to include an group. Intermediate (Ig) can be prepared using TiCl₄ method described previously. Examples of R² groups include methyl, acetyl and thiomethyl. These groups can be introduced by low temperature deprotonation of (Ig) with lithium diisopropylamine or similar base in diethyl ether and subsequent reaction with suitable electrophiles such as methyl iodide, dimethylsulfide and N-acetyl morpholine. The acetyl group can be further converted to a hydroxymethyl group under reducing conditions with a reagent such as sodium borohydride in cold methanol.

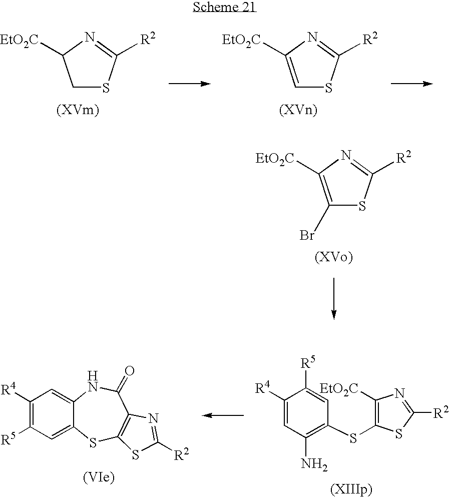

In Scheme 21 compounds of the formula (VIe) can be prepared by acid catalysed ring closure of (XIIIp) in an inert solvent such as toluene using acid catalysis by similar method to that described for (VId) in Scheme 19. The required intermediates for the generation of (VIe) can be prepared thus. Intermediate (XVm) by application of literature method as in Tetrahedron: Asymmetry 12 (2001) 1279-1286 where R² can be alkyl and include ethyl and isopropyl. The conversion to the thiazole ester intermediate (XVn) can be achieved by oxidation. Suitable oxidants include bromotichloromethane with diazabicyclo[5.4.0]undec7-ene in a suitable solvent such as dichloromethane. Conversion to the bromo ester (XVo) can be achieved by a halogenation reaction using a base in a polar aprotic solvent such as terahydrofuran at low temperature with a suitable halogenating agent. Suitable bases include lithium diisopropylamine, n-butyl lithium and the like. Halogenating agents include dibromoterachloroethane. Reaction of the bromo ester (XVo) with ortho amino thiophenol (XIVd) with a base in a suitable solvent under classic displacement conditions generate (XIIIp), as described for the corresponding chloro ester (XVi) in Scheme 18. Coupling to the various piperazine moieties is by the thioamide method as described previously.

General methods and specific examples of the synthesis of these compounds can be found in the following references:
Chakrabarti, et al., J. Med. Chem., 23, 878-884; (1980),
Chakrabarti, et al., J. Med. Chem., 23, 884-889; (1980),
Chakrabarti, et al., J. Med. Chem., 25, 1133-1140; (1982),
Chakrabarti; et al., J. Med. Chem., 32, 2573- 2582; (1989),
Liegeois, et al., J. Med. Chem., 36, 2107-2114; (1993),
Liegeois and Delarge, U.S. Pat. No. 5,393,752 (1995);
Chakrabarti and Hotten, Eur. Pat. Appl., EP 354781; (1990),
Bolton, et al., PCT Int. Appl., WO 9700252; (1997),
Chakrabarti, et al., Eur. Pat. Appl., EP 27390; (1981),
Tehim, et al., U.S. Pat. No. 5,602,124 (1998);
Tehim, et al., U.S. Pat. No. 5,824,676 (1998);
Eilingsfeld and Swybold, Ger. Offen. DE 2713573; (1978),
Gallemaers, et al., Tetrahedron Lett., 693-694; (1976),
Durnow and Abele, Chem. Ber., 97, 3349-3353, (1964),
Klempier, et al., J. Heterocyclic Chem., 29, 93-95, (1992).
Kohara, et al, Synthesis 3, 355-360 (2002).
Seio, et al, U.S. Pat. No. 6,271,225 (2001).
Schneider, U.S. Pat. No. 3,962,248 (1976).
Hunziker, U.S. Pat. No. 3,842,082 (1974).

Compounds of formula (V) of this invention may be prepared from compounds of formula (XXIV b), as shown in Scheme 22, in which one of the nitrogens in the piperazine ring may be protected by an amine protecting group, by removal of this protecting group. In this equation, ProG₂ represents an amine protecting group. Examples of such ProG₂ groups include benzyl, acetyl, t-butoxycarbonyl, methanesulfonyl, and the like. Examples of additional ProG₂ groups and methods for the introduction and removal of such groups can be found in T. W. Green, Protective Groups in Organic Synthesis, John Wiley and Sons, Inc. (1981). In the subsequent text, Pg₂ represents either hydrogen or an amine protecting group ProG₂. In the following text, for those intermediates containing a group Pg₂ in which Pg₂ is an amine protecting group, the protecting group may be removed to give the unprotected amine. Similarly, for those intermediates in which Pg₂ is hydrogen, an amine protecting group may be incorporated into the intermediate. The methods for introducing and removing these protecting groups are known in the art.

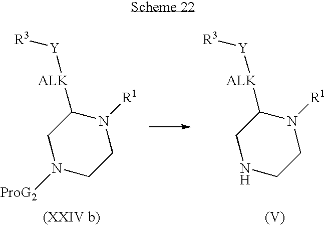

According to Scheme 23, compounds of formula (XXIV a) of this invention may be prepared from compounds of formula (XXV a) by removal of the amine protecting group ProG₁. Examples of such ProG₁ amine protecting groups include benzyl, acetyl, t-butoxycarbonyl, methanesulfonyl, and the like. Examples of additional ProG₁ groups and methods for the introduction and removal of such groups can be found in T. W. Green, Protective Groups in Organic Synthesis, John Wiley and Sons, Inc. 1981. It will be recognized that in some instances, in compounds of formula (XXV a), Pg₂ and ProG₁ may both be protecting groups that are removed under the same reaction conditions. In those cases, deprotection of this compound will yield compounds of formula (V) in which R¹ is hydrogen. In compounds of formula (XXIV a), if Pg₂ is an amine protecting group, ProG₂, then alkylation of formula (XXIV a) will yield compounds of formula (XXIV), in which R¹ is defined above.

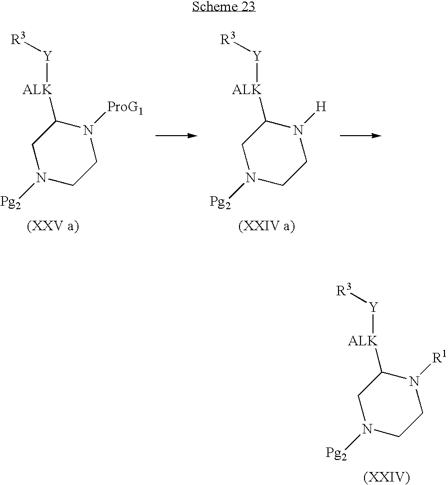

In Scheme 24, compounds of formula (XXV), in which all groups are defined as above, may be prepared by reduction of either a ketopiperazine of formula (XXVI) or a diketopiperazine of formula (XXVII). Pg₁ represents either R¹ or an amine protecting group ProG₁. Suitable reducing agents for this transformation include lithium aluminum hydride and borane. Methods for the synthesis of ketopiperazines and diketopiperazines are known in the art.

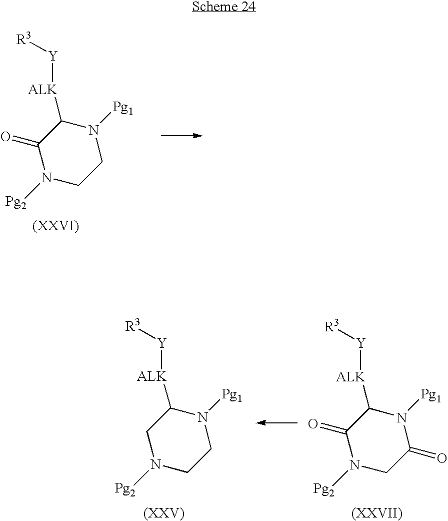

As shown in Scheme 25, compounds (XXVI) and (XXVII) may be prepared by alkylation of the corresponding ketopiperazine (XXVIII) and diketopiperazine (XXIX), respectively, with an alkylating agent of the formula Lg-R³, in which Lg is a leaving group such as a halogen, alkylsulfonyloxy, or arylsulfonyloxy group. Examples of alkylsulfonyloxy groups include methanesulfonyloxy and ethansulfonyloxy and examples of arylsulfonyloxy groups include toluenesulfonyloxy and benzenesulfonyloxy groups. This alkylation reaction is performed in the presence of a base. Suitable bases include lithium diisopropoxide, lithium hexamethyldisilazide, sodium hydride, potassium t-butoxide, and the like.

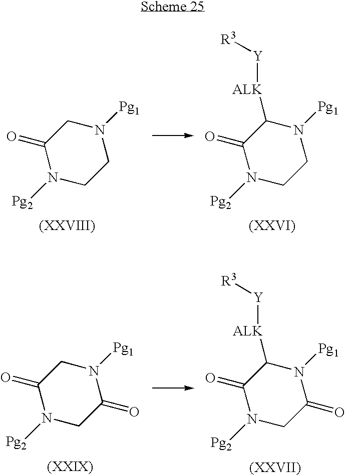

Further, as shown in Scheme 26, compounds of formula (XXVe) in which Alk is —CH₂CH₂—, —CH₂CH₂CH₂—, and —CH₂CH₂CH₂CH₂— may be prepared from a suitably protected 2-substituted piperazine of formula (XXV) by employing either a Heck coupling/reduction sequence or a hydroboration/Suzuki coupling sequence. In the Heck coupling/reduction sequence, reaction of formula (XXVb) with an arylhalide or aryl triflate in the presence of a suitable metal catalyst provides the unsaturated aryl product of formula (XXVc). For a description of the Heck reaction and its application to organic synthesis see, Whitcombe, et al., Tetrahedron, 57, 7449-7476, (2001); Herrmann, Wolfgang A., Appl. Homogeneous Catal. Organomet. Compd., 2, 712-732, VCH, Weinheim, Germany (Editors: Cornils, Boy; Herrmann, Wolfgang A.), (1996); and references cited therein. Reduction of compounds of formula (XXVc) provides compounds of formula (XXV b), m=0-2. Suitable reducing conditions include catalytic hydrogenation.

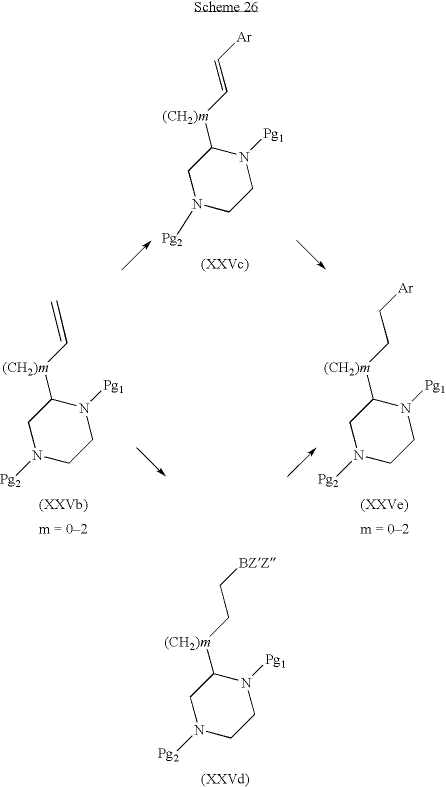

The hydroboration/Suzuki coupling sequence represents a second method for converting compounds of formula (XXVb) to compounds of formula (XXV e). Reaction of formula (XXVb) with a borane HBZ′Z″, in which Z′ and Z″ are independently H, alkyl such as methyl, ethyl, propyl, or alkoxy such as methoxy, ethoxy, or propoxy provides an organoborane of formula (XXVd). Suitable boranes HBZ′Z″ include, borane, trisiamylborane, catecholborane, and 9-borabicyclo[3,3,0]nonane (9-BBN). Reaction of formula (XXVd) with an arylhalide or aryl triflate in the presence of a suitable catalyst provides compounds of formula (XXVe). For a description of the Suzuki reaction and its application to organic synthesis see, Miyaura and Suzuki, Chem. Rev., 95, 2457-2483, (1995), and references cited therein.

Compounds of formula (XXVb) (m=0) may be prepared by the method described in Tsuda, et al., J. Org. Chem., 55, 3388-3390, (1990), and Uozumi, et al., J. Org. Chem., 58, 6826-6832, (1993).

In Scheme 27 compounds of formula (XXVb) (m=1, 2) may be prepared by an alkylation of formula (XXVIII) with an allyl halide or a homoallyl halide and to give compounds of formula (XXVIa) and reduction with lithium aluminum hydride to give compounds of formula (XXVb) (m=1, 2).

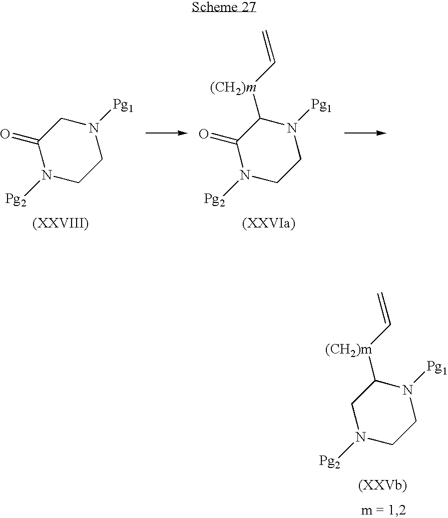

Further, as shown in Scheme 28, compounds of formula (XXVf) in which Alk is —CH₂CH₂—, —CH₂CH₂CH₂—, and —CH₂CH₂CH₂CH₂— may be prepared from a suitably protected 2-substituted piperazine of formula (XXVb) by employing a hydroboration/oxidation sequence as described previously. The resulting organoborane is then oxidized to the alcohol (XXVf) using an oxidant such as hydrogen peroxide or t-butylhydroperoxide.

Compounds of formula (XXVg) may be formed from compounds (XXVb) by hydration of the olefin. This hydration is typically performed under acidic conditions or may also be performed through an oxymercuration/reduction sequence. The oxymercuration is typically performed by treatment of the olefin with a mercury(II) salt such as Hg(OAc)₂. The mercury atom is removed from the intermediate compound through reduction with NaBH₄.

It should be noted that compounds (XXVf) and (XXVg) are regioisomers of one another. Mixtures of these compounds can result from either the hydroboration/oxidation, acid catalyzed hydration, or oxymercuration/reduction sequences if the regiochemical control of these processes is limited.

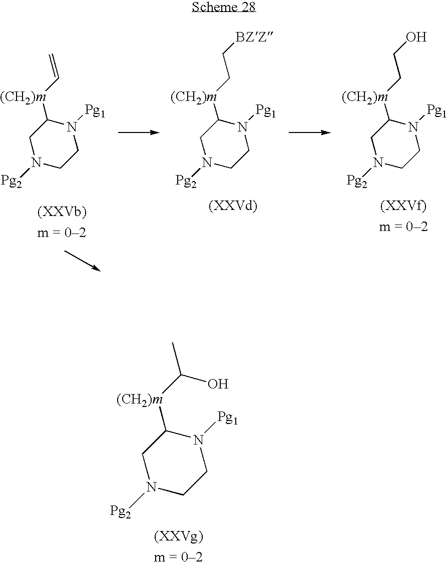

As shown in Scheme, 29, compounds of the formula (XXVf) (m=0, 1) and (XXVg) (m=0,1) may be oxidized to the corresponding aldehyde (XXVh) (m=0, 1) and ketone (XXVi) (m=0, 1), respectively. Suitable oxidizing reagents include pyridinium chlorochromate, DMSO/oxalyl chloride (Swern oxidation) and dimethylsulfide/N-chlorosuccinimide (Corey-Kim oxidation). Compounds (XXVh) and (XXVi) may be treated with an organoalkyl reagent, MR³ to provide alcohols (XXVj) (m=0, 1) and (XXVk) (m=0, 1), respectively. Suitable organoalkyl reagents include organolithium reagents such as methyllithium and ethyllithium, Grignard reagents such as methylmagnesium bromide and ethylmagnesium chloride, and the like.

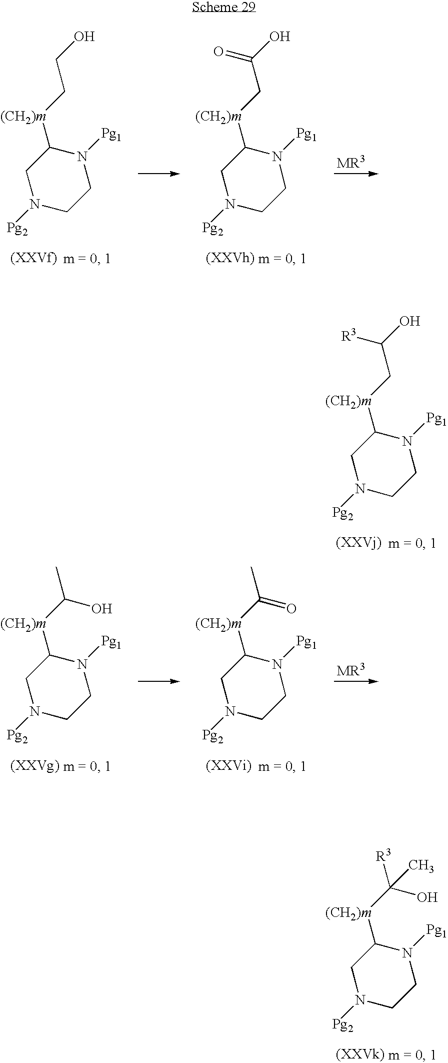

As shown in Scheme 30, an alcohol of the formula (XXV l) may be transformed to the corresponding ethers and thioethers (XXV m) (Y=O, S) and (XXV o) through a number of methods. The oxygen of alcohol (XXV l) may be treated with an alkylating agent to form ether (XXV m) in which R³ is an alkyl group. Suitable alkylating agents include dimethyl sulfate, alkyl halides such as methyl iodide, ethyl bromide, and benzyl chloride, and sulfonate esters such as methyl tosylate, ethyl methanesulfonate, and methyl trifluoromethanesulfonate. This alkylation is usually performed under basic conditions.

Alternatively as depicted in Scheme 30, compound (XXV l) may be converted into a compound of structure (XXVn) in which Lg is a leaving group. Examples of leaving groups Lg include halogen, the alkylsulfonyloxy group, and the arylsulfonyloxy group. Examples of alkylsulfonyloxy groups include methanesulfonyloxy and ethansulfonyloxy and examples of arylsulfonyloxy groups include toluenesulfonyloxy and benzenesulfonyloxy groups. Compounds in which Lg is a halogen such as chlorine or bromine may be prepared from (XXV l) by reaction with an inorganic halide such as thionyl chloride, phosphorus pentachloride, or phosphorus tribromide. Compounds in which Lg is an alkylsulfonyloxy group or arylsulfonyloxy group may be prepared by reaction of (XXV l) with the corresponding alkylsulfonyl halide, arylsulfonyl halide, alkylsulfonic anhydride or arylsulfonic anhydride in the presence of a base. Reaction of (XXVn) with an alcohol R³OH or a thiol R³SH provides the corresponding ethers and thioethers (XXV o) (Y=O, S). This reaction is typically performed under basic conditions in an inert solvent. Suitable bases include sodium hydride, sodium hydroxide, and potassium hydride.

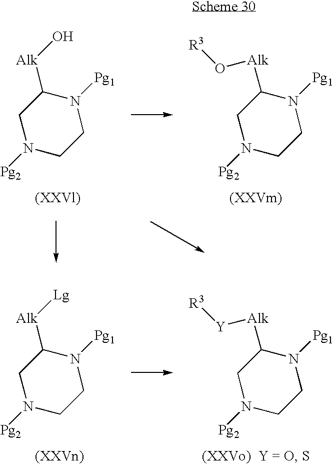

Alternatively, as also shown in Scheme 32, (XXV l) may be converted directly into (XXV o) (Y=O, S) by treatment with an alcohol R₃OH or thiol R₃SH under Mitsunobu conditions. Classical Mitsunobu conditions employ triphenylphosphine and diethyl azodicarboxylate. The Mitsunobu reaction has been reviewed in the following references:

David L. Hughes, Organic Reactions, 42, 335-656 (1992);

David L. Hughes, Organic Preparations and Procedures International, 28, 127-164 (1996).

As shown in Scheme 31, thioethers (XXV p) may be converted into the corresponding sulfoxides, (XXV q) m=1, and sulfones, (XXV q) m=2, by reaction with an appropriate oxidizing agent. Oxidizing agents include molecular oxygen, hydrogen peroxide, t-butyl hydroperoxide, peroxyacetic acid, meta-chloroperoxybenzoic acid, ozone, and oxone (potassium peroxymonosulfate).

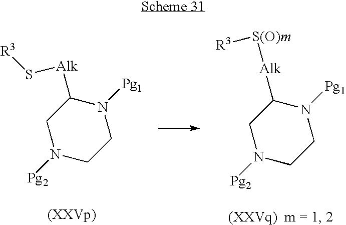

The skilled artisan will appreciate that many of the aforementioned reactions may be performed in any convenient order. Similarly, for those compounds that contain an asymmetric center, the skilled artisan will recognize that the aforementioned reactions may be performed either on pure isomers or on a mixture of isomers. The isomers may be separated at any convenient stage during the synthesis.

Pharmacological Activity

Compounds of the formula (I) have moderate to high binding affinity for multiple neurotransmitter receptors, and in particular, the dopamine receptors. Those skilled in neuropharmacology and related disciplines have recognized dopamine receptor binding activity as indicative of antipsychotic, in particular, antischizophrenic properties. See P. Seeman, et al., Nature, 261, 717-718 (1976); P. Seeman, Synapse, 1, 133 (1987); H. Howard, et al., 28, 39 (1993); and J. Schaus. et al., Annual Reports in Medicinal Chemistry, 33, 1 (1998). Cloning studies have currently demonstrated five principal dopamine receptor subtypes that fall into two major classes, D₁-like and D₂-like. The D₁-like class includes the D₁ and D₅ subtypes, and the D₂-like class encompasses the D₂, D₃, and D₄ subtypes. The experimental protocol for the assay generating this data is in the Example section below. Thus, many of the compounds of formula (I) exhibit D₂ receptor affinity greater than both clozapine and olanzapine.

Like clozapine and olanzapine, the compounds of formula (I) also exhibit high affinity for the 5-HT₆ receptor. Because clozapine and olanzapine have greater efficacy in treating the cognitive disturbances of schizophrenia (Purdon, et al., Arch. Gen. Psych., 57, 249 (2000)) and selective 5-HT₆ antagonists are active in models of cognitive enhancement, this activity is desirable in an antipsychotic drug.

Many atypical antipsychotics have a high affinity for the 5-HT_{2A} receptor. Researchers believe that high affinity for the 5-HT_{2A} receptor helps in treating the negative symptoms of schizophrenia and preventing some of the motor side effects (H Meltzer, et al., J. Pharm. Exp. Ther. 25, 238 (1989)). However, selective 5-HT_{2A} antagonists are not effective antipsychotics as monotherapy. Thus, 5-HT_{2A} antagonism would likely be among the other neuroreceptor affnities of a superior antipsychotic compound. The compounds of formula (I) exhibit a desirable level of 5-HT_{2A} affinity.

Antipsychotics are believed to exert at least part of their therapeutic effects through blockade of the dopamine D₂ receptor. The ability of a compound to block dopamine D₂ receptors in the rat in vivo was determined by measuring the effect of the compound on the level of DOPAC (3,4-dihydroxyphenylacetic acid), a metabolite of dopamine, in nucleus accumbeus of the rat. Dopamine D₂ receptor antagonists increase the release of dopamine into the synapse due to blockade of the dopamine D₂ autoreceptor. This increased release of dopamine cannot be directly measured, since the efficiency of the dopamine reuptake system prevents increases in synaptic dopamine concentrations. Instead, increases in the levels of the dopamine metabolites DOPAC (3,4-dihydroxyphenylacetic acid) and HVA (homovanillic acid) reflect increased neuronal dopaminergic activity in vivo. For example, olanzapine and other dopamine D₂ receptor antagonists increase concentrations of DOPAC and HVA in striatum and nucleus accumbens without appreciable alteration of dopamine concentrations. The potency of a compound to block dopamine D₂ receptors was determined by the dose required to increase DOPAC levels to 200% of control. This value is called the ED₂₀₀.

Antipsychotics are believed to induce at least part of their weight gain effects through blockade of histamine H₁ receptors in the hypothalamus.

Their ability of a compound to block histamine H₁ receptors can be estimated in vitro by measuring the in vitro histamine H₁ receptor affinity. Compounds with decreased affinity for histamine H₁ receptors will be less likely to induce weight gain. The ratio of in vitro histamine H₁ receptor affinity divided by the in vitro dopamine D₂ receptor affinity, both expressed as Ki's, is an estimate of a compound's likelihood to cause weight gain at therapeutic levels. The greater this ratio, the less likely a compound will be to cause weight gain. The ratios of clozapine and olanzapine are 0.01 and 0.3, respectively. Compounds of this invention have H₁/D₂ ratios greater than or equal to 0.1. Compounds of this invention preferably have H₁/D₂ ratios greater than or equal to 1 and more preferably H₁/D₂ ratios greater than or equal to 3.

The in vivo potency of a compound to occupy hypothalamic histamine H₁ receptors in the rat was determined using a histamine H₁ ex vivo binding assay. The ED₅₀ is the dose required to occupy 50% of the rat histamine H₁ receptors. The greater the ED₅₀ the less likely it will be that a compound will cause weight gain. The compounds of this invention preferably have histamine H₁ ex vivo binding ED₅₀ greater that or equal to 10 mg/kg,po and more preferably have ED₅₀'s greater that 30 mg/kg,po.

### Histamine H₁ Ex Vivo Binding and DOPAC Concentrations

### Methods

Male Sprague Dawley rats (Harlan Sprague Dawley, Inc., Indianapolis, Ind.) weighing 110 grams were fasted overnight. Animals were gavaged with clozapine (RBI, Inc.) or with the compound of interest and sacrificed 90 minutes later. Clozapine was administered at 5 ml/kg in 5% acacia suspension. All other compounds were administered at 5 ml/kg in dilute lactic acid. Tissues were dissected, frozen on dry ice and stored at −70° C. prior to analysis.

### Histamine H₁ Ex Vivo Binding

Ex vivo binding of the histamine H₁ antagonist [³H]-pyrilamine (NEN Life Science Products) to rat hypothalamic homogenates was determined. Tissues were homogenized in 600 μl incubation buffer (50 mM sodium phosphate monobasic, pH 7.4) and pre-incubated 10 minutes at 37° C. to remove endogenous histamine. Triplicate tubes, each containing 100 μl homogenate, were combined with 1 ml buffer containing 3 nM [³H]-pyrilamine and incubated 30 minutes at 25° C. Non-specific tissue binding was also measured in duplicate in tubes containing 10 μM clozapine. [³H]-pyrilamine binding was measured after separation filtration using a Brandell cell harvester with GF/C filters which had been soaked in 0.1% polyethylenimine. ED₅₀ values were determined using the Allfit statistical program for displacement binding.

### DOPAC Concentrations

Rat nucleus accumbens DOPAC (3,4-dihydroxyphenylacetic acid) concentrations were measured using high-pressure liquid chromatography with electrochemical detection (HPLC-EC). Tissues were sonicated in 1 ml 0.1N TCA. After centrifugation, a 25 μl aliquot of supernatant was injected onto a BDS Hypersyl C18 column (150×4.6 mm, Keystone Scientific). The elution buffer contained 75 mM sodium phosphate monobasic, 0.5 mM EDTA, 350 mg/L 1-octanesulfonate sodium, 7% acetonitrile (v/v) and 0.7% tetrahydrofiran (v/v), pH 3.0. The flow rate was 1.2 m/min at 40° C. Peak heights were measured at 750 mV at 10 nA sensitivity and compared with samples containing known amounts of DOPAC standards. Doses that increased DOPAC levels to 200% of control values (ED₂₀₀'s) were calculated using a best-fit linear regression analysis.

The compounds of formula (I) are useful for treating pathologic psychologic conditions, especially psychosis, with minimal detrimental adverse events. Pathologic psychological conditions which are psychosis or may be associated with psychotic features include, but are not limited to the psychotic disorders which have been characterized in the DSM-IV-TR., Diagnostic and Statistical Manual of Mental Disorders. Revised, 4^{th} Ed., Text Revision (2000). See also DSM-IV, Diagnostic and Statistical Manual of Mental Disorders 4^{th} Ed., (1994). The DSM-IV and DSM-IV-TR was prepared by the Task Force on Nomenclature and Statistics of the American Psychiatric Association, and provides descriptions of diagnostic categories. The skilled artisan will recognize that there are alternative nomenclatures, nosologies, and classification systems for pathologic psychological conditions and that these systems evolve with medical scientific progress. Examples of pathologic conditions associated with psychosis that may be treated with the compounds of the present invention include, but are not limited to, schizophrenia, schizophreniform disorder, schizoaffective disorder, delusional disorder, brief psychotic disorder, shared psychotic disorder, psychotic disorder due to a general medical condition, substance-induced psychotic disorder, schizotypical, schizoid, paranoid personality disorder, and psychotic disorder-not other specified, see DSM-IV, Section: Schizophrenia and Other Psychotic Disorders, pages 273 to316.

Compounds of the present invention are useful in treating depression and mood disorders found in the DSM-IV, Diagnostic and Statistical Manual of Mental Disorders 4^{th} Ed., (1994) Section: Mood Disorders, pages 317 to 392. Disorders include, but are not limited to, mood disorders such as major depressive episodes, manic episode, mixed episode, hypomanic episode; depressive disorders such as major depressive disorder, dysthymic disorder, depressive disorder not otherwise specificed; Bipolar disorders such as bipolar I disorder, bipolar II disorder, cyclothymic disorder, bipolar disorder not otherwise specified; other mood disorders such as mood disorder due to general medical conditions, substance-induced mood disorder, mood disorder not otherwise specified; and mood disorders with mild, moderate, severe without psychotic features, severe with psychotic features, in partial remission, in fuill remission, with catatonic features, with melancholic features, with atypical features, with postpartum onset.

One of oridinary skilled in the art would appreciate that the compounds of the present invention would be useful in the treatment of depressive episodes associated with bipolar disorders, treatment of manic episodes associated with bipolar disorders such as, but not limited to, the treatment of the acute manic episodes associated with bipolar I disorder.

Compounds of the present invention are useful in treating cognitive disorders, age-related cognitive disorder, mild cognitive impairment, postconcussional disorder, mild neurocognitive disorder, anxiety (particularly including generalized anxiety disorder, panic disorder, and obsessive compulsive disorder), and migraine (including migraine headache). These compounds are also useful in treating substance withdrawal (including substances such as opiates, nicotine, tobacco products, alcohol, benzodiazepines, cocaine, sedatives, hypnotics, caffeine, etc.). Other conditions that may be treated with the compounds of the present invention include, but are not limited to, dementia, dementia with behavioral disturbances, movement disorders, personality disorders, borderline personality disorder, pervasive development disodeis, eating disorders, premenstrual dysphoric disorder, tic disorders, sexual dysfunction, delirium, emesis, substance related disorders, impulse-control disorders, postpsychotic depressive disorder of schizophrenia, simple deteriorative disorder (simple schizophrenia), minor depressive disorder, recurrent brief depressive disorder, and mixed anxiety-depresssive disorder.

Compounds of the present invention are also useful in treating the cognitive deficients associated with the above listed, but not limited to, psychological conditions such as schizophrenia, mood disorders, and other psychotic disorders.

An effective amount can be readily determined by the attending diagnostician, as one skilled in the art, by the use of known techniques and by observing results obtained under analogous circumstances. In determining the effective amount or dose, a number of factors are considered by the attending diagnostician, including, but not limited to: the species of mammal; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

The compounds of the present invention are effective over a wide dosage range, but the actual dose administered being dependent on the condition being treated. While the exact dose is administered according to the discretion of the attending health care professional, typically, in the treatment of adult humans, dosages of from 0.1 to 500 mg, preferably from 0.25 mg to 100 mg, most preferably 0.25 mg to 50 mg per day may be used. A once a day dosage is normally sufficient, although divided doses may be administered. For example, for the treatment of psychotic disorders a dose range of from 0.1 mg to 500 mg, preferably 0.25 mg to 100 mg, per day is suitable.

In choosing a suitable regimen for patients suffering from psychotic conditions, compositions containing compounds of formula (I) as an active ingredient may be formulated to provide quick, sustained or delayed release of the active ingredient after administration to the patient. Depending on the method of administration, compositions may be formulated as tablets, capsules, suspensions, or elixirs for oral use, or injection solutions or suppositories for parental use. Preferably the compositions are formulated in a unit dosage form, each dosage containing from 0.1 mg to 500 mg, more usually 0.25 mg to 100 mg, of the active ingredient.

A preferred formulation of the invention is a capsule or tablet comprising 0.1 to 500 mg of active ingredient together with a pharmaceutically acceptable carrier. A further preferred formulation is an injection which in unit dosage form comprises 0.1 mg to 500 mg of active ingredient together with a pharmaceutically acceptable diluent. A sustained release formulation is also a preferred formulation.

Pharmaceutical Formulations

While it is possible to administer a compound of formula (I) with no additional ingredients to a patient in need thereof, if is far more desirable to administer such a compound in the form of a pharmaceutical composition. Pharmaceutical compositions containing a compound of formula (I) as an active ingredient provides control of the dosage and rate of absorption into the body and stability of the product in shipment and storage. Further, pharmaceutical formulations are more acceptable to the patient being treated, and thus increase compliance with a treatment program. Such compositions, comprising at least one pharmaceutically acceptable carrier, are valuable and novel because of the presence of the compounds of formula (I) therein. Formulation of pharmaceutical compositions is an art unto itself, about which much has been published. The compounds of the present invention may be formulated into pharmaceutical compositions by essentially any suitable method of the art including, but not limited to, the methods discussed hereinbelow.

The usual methods of formulation used in pharmaceutical science and the usual types of compositions may be used, including tablets, chewable tablets, capsules, solutions, parenteral solutions, intranasal sprays or powders, troches, suppositories, transdermal patches and suspensions. In general, compositions contain from about 0.5% to about 50% of the compound in total, depending on the desired dose and the type of composition to be used. The amount of the compound, however, is best defined as the effective amount, that is, the amount of each compound which provides the desired dose to the patient in need of such treatment. The activities of the compounds do not depend on the nature of the composition, so the compositions are chosen and formulated solely for convenience and economy. Any compound may be formulated in any desired form of composition. Some discussion of different compositions will be provided, followed by some typical formulations.

Capsules are prepared by mixing the compound with a suitable diluent and filling the proper amount of the mixture in capsules. The usual diluents include inert powdered substances such as starch of many different kinds, powdered cellulose, especially crystalline and microcrystalline cellulose, sugars such as fructose, mannitol and sucrose, grain flours and similar edible powders.

Tablets are prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant is necessary in a tablet formulation to prevent the tablet and punches from sticking in the die. The lubricant is chosen from such slippery solids as talc, magnesium and calcium stearate, stearic acid and hydrogenated vegetable oils.

Tablet disintegrators are substances which swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethylcellulose, for example, may be used, as well as sodium lauryl sulfate.

Enteric formulations are often used to protect an active ingredient from the strongly acidic contents of the stomach. Such formulations are created by coating a solid dosage form with a film of a polymer which is insoluble in acidic environments, and soluble in basic environments. Exemplary films are cellulose acetate phthalate, polyvinyl acetate phthalate, hydroxypropyl methylcellulose phthalate and-hydroxypropyl methylcellulose acetate succinate.

Tablets are often coated with sugar as a flavor and sealant, or with film-forming protecting agents to modify the dissolution properties of the tablet. The compounds may also be formulated as chewable tablets, by using large amounts of pleasant-tasting substances such as mannitol in the formulation, as is now well-established practice. Instantly dissolving tablet-like formulations are also now frequently used to assure that the patient consumes the dosage form, and to avoid the difficulty in swallowing solid objects that bothers some patients.

When it is desired to administer the combination as a suppository, the usual bases may be used. Cocoa butter is a traditional suppository base, which may be modified by addition of waxes to raise its melting point slightly. Water-miscible suppository bases comprising, particularly, polyethylene glycols of various molecular weights are in wide use, also.

Transdermal patches have become popular in recent years because of technological advances in matrix compositions. Typically they comprise a resinous matrix composition in which the drugs will dissolve, or partially dissolve, which is held in contact with the skin by a film which protects the composition. Many patents have appeared in the field recently. Other, more complicated patch compositions are also in use, particularly those having a membrane pierced with innumerable pores through which the drugs are pumped by osmotic action.

### EXAMPLES

The following examples illustrate aspects of this invention but should not be construed as limitations thereof. The symbols and conventions used in these examples and consistent with those used in the contemporary chemical literature such as the Journal of the American Chemical Society, and Tetrahedron Letters, and contemporary literature of other scientific disciplines as appropriate.

### Example 1

### 2-Methyl-4,9-dihydro-3-thia-9-aza-benzo[f]azulen-10-one

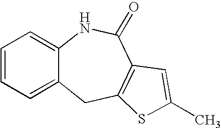

Prepared according to Eur. J. Med. Chem. 1981 p. 391-398.

### Example 2

### 2-Methyl-9H-3-thia-9-aza-benzo[f]azulene-4,10-dione

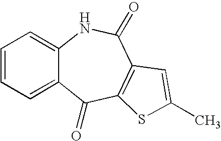

Dissolve 2-methyl-4,9-dihydro-3-thia-9-aza-benzo[f]azulen-10-one (1.10 g, 4.80 mmol) in glacial acetic acid with chromium trioxide (2.88 g, 28.81 mmol). Heat the resulting mixture to reflux (118° C.) for five hours. Cool reaction mixture to ambient temperature and neutralize (pH 6-8) using saturated aqueous sodium bicarbonate. Extract three times with ethyl acetate; combine organic layers, dry over sodium sulfate, and concentrate under reduced pressure to give a residue. Purification of the residue by flash chromatography eluting with 97% dichloromethane:3% 2 M ammonia in methanol, gives the title compound: Mass Spectrum (m/e): 244.0(M+1).

### Example 3

### 2-Isopropyl-4,9-dihydro-3-thia-9-aza-benzo[f]azulen-10-one

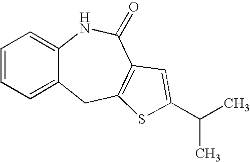

Prepared according to Eur. J. Med. Chem. 1981 p. 391-398, using 2-isopropyl thiophene in place of 2-methyl thiophene.

### Example 4

### Benzo[b]thiophen-2-yl-(5-fluoro-2-nitro-phenyl)-methanol

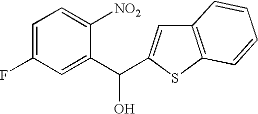

Dissolve thianaphthen (1.35 g, 10.06 mmol) in THF (20 ml) at −78° C. and add dropwise n-BuLi (6.9 ml, 11.07 mmol), stir the reaction mixture −78° C. After 1 hour, add 5-fluoro-2-nitrobenzoaldehyde (2.0 g, 12.03 mmol) in THF (10 ml). Stir the reaction mixture at −78° C. for an additional 2 hours and warm to room temperature. Stir the reaction mixture at room temperature. After 12 hours, treat the reaction mixture with acetone (20 ml) followed by H₂O (50 ml). Separate the organic layer, wash with brine and dry (MgSO₄). Purification by flash chromatography, eluting with ethyl acetate/hexane (20%) to give the title compound: Mass spectrum (m/e): 304.0 (M+1).

### Example 5

### Benzo[b]thiohen-2-yl-(5-chloro-2-nitro-phenyl)-methanol

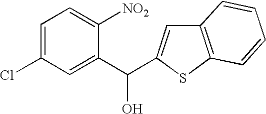

By using a method similar to Example 4, the title compound is prepared: mass spectrum (m/e): 320.5(M+1).

### Example 6

### Benzo[b]thionhen-2-yl-(4-chloro-2-nitro-phenyl)-methanol

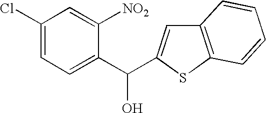

By using a method similar to Example 4 the title compound is prepared: mass spectrum (m/e): 320.5(M+1).

### Example 7

### Benzo[b]thiophen-2-yl-(2-nitro-phenyl)-methanol

By using a method similar to Example 4, the title compound is prepared.

### Example 8

### (2-Amino-phenyl)-benzo[b]thiophen-2-yl-methanone

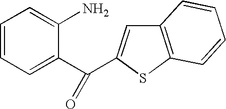

Combine thianaphene (11.36, 84.6 mmol) in dry ether (200 ml) at −78° C. and add n-BuLi (52.8 ml, 84.6 mmol) and stir the reaction mixture at −78° C. for 30 min and warm to room temperature for 1 hour. Cool the reaction mixture to −78° C., treat with 2-aminobenzonitrile in dry ether (100 ml) and stir the reaction mixture at −78° C. After 2 hours, warm to room temperature. Allow the reaction to stir at room temperature for an additional 18 hours. Pour the reaction mixture into 5N HCl (200 ml) and stir for 30 min. Separate the two layers wash the organic layer with brine and dry (Mg SO₄). Purification by flash chromatography, eluting with ethyl acetate/ hexane (20%) to give the title compound: mass spectrum (m/e): 254.8(M+1).

### Example 9

### Benzo[b]thiophen-2-yl-(5-fluoro-2-nitro-phenyl)-methanone

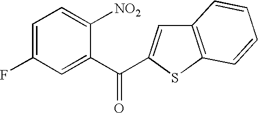

Dissolve (2-amino-5-fluoro-phenyl)-benzo[b]thiophen-2-yl-methanol (0.8 g, 2.6 mmol) in dichloromethane (100 ml) and add pyridinium dichloromate (1.49 g, 3.96 mmol) and stir at room temperature. After 18 hours, filter the reaction mixture through a MgSO₄ pad, filtrate evaporate under reduced pressure to give the title compound: mass spectrum (m/e): 362.0 ( M+1).

### Example 10

### Benzo[b]thiophen-2-yl-(5-chloro-2-nitro-phenyl)-methanone

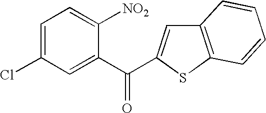

By using a method similar to Example 9, the title compound is prepared: mass spectrum (m/e): 318.5(M+1).

### Example 11

### Benzo[b]thiophen-2-yl-4-chloro-2-nitro-phenyl)-methanone

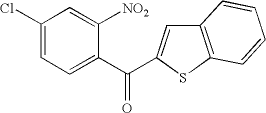

By using a method similar to Example 9, the title compound is prepared: mass spectrum (m/e): 318.5(M+1).

### Example 12

### 2-Benzo[b]thiophen-2-ylmethyl-phenylamine

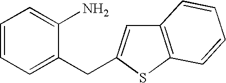

Suspend (2-amino-phenyl)-benzo[b]thiophen-2-yl-methanone (0.85 g, 3.35 mmol) in acetic acid (20 ml) at room temperature, and add zinc powder (4.3 g, 67.1 mmol) and heat the reaction mixture to 115° C. After 3 hours, cool the reaction mixture to room temperature and filter, evaporate the filtrate under reduced pressure to give 2-benzo[b]thiophen-2-ylmethyl-phenylamine as an oil.

### Example 13

### 2-Benzo[b]thiophen-2-ylmethyl-4-fluoro-phenylamine

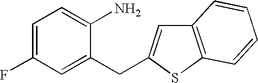

By using a method similar to Example 12, the title compound was prepared: mass spectrum (m/e): 258.1(M+1).

### Example 14

### 2-Benzo[b]thiophen-2-ylmethyl-4-chloro-phenylamine

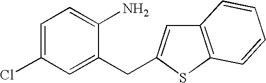

By using a method similar to Example 12, the title compound was prepared: mass spectrum (m/e): 274.7 (M+1).

### Example 15

### 2-Benzo[b]thiophen-2-ylmethyl-4-chloro-phenylamine

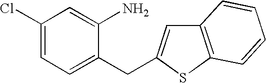

By using a method similar to Example 12, the title compound was prepared: mass spectrum (m/e): 274.8(M+1).

### Example 16

### 4-R¹-(Alk-Y-R³-piperazine)-1-carboxylic acid-(2-benzo[b]thiophen-2-ylmethyl-phenyl)-amide

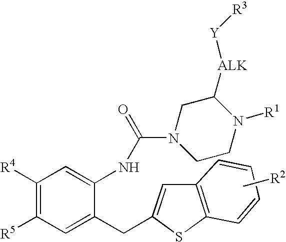

Dissolve the appropriate 2-benzo[b]thiophen-2-ylmethyl-phenylamine (0.25 g, 1.05 mmol) in toluene (10 ml), and add 20% phosgene in toluene (2.0 ml) and reflux the reaction mixture. After 3 hours, evaporate the reaction mixture under reduced pressure. Dissolve the residue in acetonitrile (10 ml), and add the appropriate piperazine (9.5 to 10 equiv) and reflux the reaction mixture. After 18 hours, cool the reaction mixture to room temperature and evaporate under reduced pressure. Purification by flash chromatography, eluting with dichloromethane/dichloromethane:methanol:ammonia (94%:6%:0.35 M gradient) to give the amide. Treating the resulting oil in ethyl acetate with 1 N HCl in ether (0.5 ml) and concentrating under reduced pressure gives the appropriate compound.

Using the method. of Example 16 gives the following compounds, isolated as the free base except where noted:

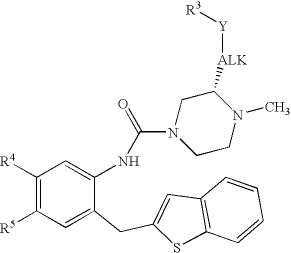

| NO. | Alk | Y | R³ | R⁴ | R⁵ | DATA |
|---|---|---|---|---|---|---|
| 20 | CH₂CH₂ | O | CH₃ | H | F | Mass spectrum (m/e): 424.2 |
| 21 | CH₂CH₂ | O | CH₃ | H | Cl | Mass spectrum (m/e): 459.1 |
| 21a | CH₂CH₂ | O | CH₃ | F | F | Mass spectrum (m/e): 442.1 |

### Example 22

### 5-[Hydroxy-(2-nitro-phenyl)-methyl]-2-methyl-thiazole-4-carbonitrile

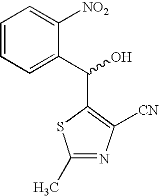

Dissolve 2-methyl-thiazole-4-carbonitrile (3.50 g, 28.19 mmol) in 200 ml of tetrahydrofuran (THF) and cool in a dry ice/acetone bath for 1.5 hours. Slowly add n-butyllithium (26.4 ml of a 1.6 M solution in hexanes, 42.28 mmol) and stir for one hour. Add a THF solution of 2-nitrobenzaldehyde (4.69 g, 31.01 mmol, in 50 ml of THF) and stir in cold bath for 12 hours. Allow the reaction mixture to warm to room temperature, and add 100 ml of saturated aqueous ammonium chloride to quench the reaction. Extract three times with ethyl acetate, combine organic layers and dry over sodium sulfate. Remove solvent under reduced pressure. Purification via flash chromatography, eluting with a step gradient starting with hexanes and going to 75% hexanes with 25% ethyl acetate, gives the title compound: Mass Spectrum (m/e): 276(M+1).

### Example 23

### 2-Methyl-5-(2-nitro-benzoyl)-thiazole-4-carbonitrile

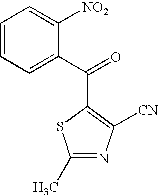

Dissolve 5-[hydroxy-(2-nitro-phenyl)-methyl]-2-methyl-thiazole-4-carbonitrile (3.33 g, 12.10 mmol) in dichloromethane (150 ml) and add molecular sieves (4 angstrom). Add pyridinium dichromate (6.83 g, 18.15 mmol). Stir the mixture at ambient temperature for 16 hours. Filter the mixture through a pad of magnesium sulfate, and wash the pad with dichloromethane. Collect the filtrate and remove the solvent under reduced pressure to afford the title compound (3.15 g, 11.53 mmol, 95% yield) as a yellow, amorphous solid: Mass Spectrum (m/e): 272(M−1) (Title compound only seen in negative electrospray).

### Example 24

### 10-Amino-2-methyl-3-thia-1,9-diaza-benzo[f]azulen-4-one hvdrochloride

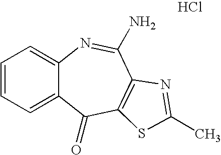

Dissolve 2-methyl-5-(2-nitro-benzoyl)-thiazole-4-carbonitrile (3.13 g, 11.45 mmol) in 4 M hydrochloric acid in 1,4-dioxane (150 ml). Add to the stirring mixture tin (II) chloride (6.51 g,34.36 mmol) and stir the resulting mixture for 16 hours at ambient temperature. Remove the solvent under reduced pressure. Pour hydrochloric acid (5.00 N, 150 ml) in with the residue and stir the slurry for two hours at ambient temperature. Filter the slurry and collect solid and dry under vacuum to give the title compound: Mass Spectrum (m/e): 244(M+1) (Title compound shows up in mass spectrum as the free base).

### Example 25

### 2-Methyl-9H-3-thia-1,9-diaza-benzo[f]azulene-4,10-dione

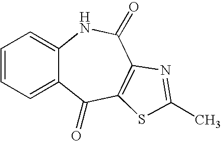

Dissolve 10-amino-2-methyl-3-thia-1,9-diaza-benzo[f]azulen-4-one hydrochloride (10 g, 3.75 mmol) in concentrated hydrochloric acid (70 ml). Heat the mixture to 100° C. for 16 hours. Cool mixture to ambient temperature and extract three times with a 3:1 mixture of chloroform: isopropyl alcohol. Remove solvent under reduced pressure. Purification via flash chromatography, eluting with a step gradient starting with dichloromethane and going to 95% dichloromethane with 5% 2 M ammonia in methanol, gives the title compound: Mass Spectrum (m/e): 245(M+1).

### Example 26

### 2-Methyl-4,9-dihydro-3-thia-1,9-diaza-benzo[f]azulen-10-one

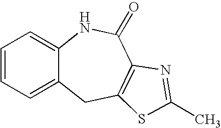

Dissolve 2-methyl-9H-3-thia-1,9-diaza-benzo[f]azulene-4,10-dione (0.446 g, 1.83 mmol) in glacial acetic acid (25 ml). Slowly add zinc powder (2.39 g, 36.52 mmol) in portions with stirring. Heat the mixture to 100° C. for four hours. Filter the reaction mixture while it is still hot, and wash the filter cake with acetic acid. Collect filtrate and concentrate under reduced pressure. Take residue up in ethyl acetate and wash once with water, then twice with brine. Collect organic layer and dry over sodium sulfate, remove solvent under reduced pressure. Purification of the residue by flash chromatography, eluting with a step gradient starting with dichloromethane and going to 97% dichloromethane with 3% 2 M ammonia in methanol, gives the title compound: Mass Spectrum (m/e): 231(M+1).

### Example 27

### 2-Methyl-4,9-dihydro-3-thia-1,9-diaza-benzo[f]azulene-10-thione

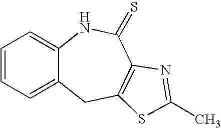

Slurry 2-methyl-4,9-dihydro-3-thia-1,9-diaza-benzo[f]azulen-10-one (0.310 g, 1.35 mmol) and Lawesson's reagent [2,4-bis(4-methoxyphenyl)-1,3-dithia-2,4-diphosphetane-2,4-disulfide] (0.408 g, 0.75 mmol) in 1,2 dichoroethane and heat to reflux (83° C.) for 2 hours. Cool reaction mixture to ambient temperature, then concentrate under reduced pressure. Purification of the residue via flash chromatography, eluting with a step gradient starting with 95% toluene with 5% THF going to 90% toluene with 10% THF, gives the title compound: Mass Spectrum (m/e): 247(M+1).

### Example 28

### 2-Isopropyl-thiazolidine-4-carboxylic acid methyl ester

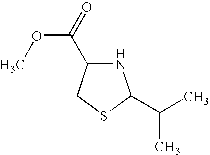

Add to a solution of L-cysteine methyl ester in a mixture of ethanol-water (200 ml, 1:1) KOAc followed by isobutylaldehyde (7.3 g, 101.6 mmol). Stir the resulting solution at room temperature. After 4 hours, evaporate the reaction mixture under reduced pressure. Take up the residue with ether (300 ml), wash with brine, and evaporate under reduced pressure to give the title compound.

Using the method of Example 28 gives the following compounds, isolated as the free base except where noted:

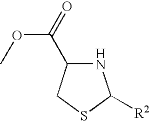

| NO.: | R² | DATA |
|---|---|---|
| 29 | Tert-butyl | mass spectrum (m/e): 204.1 (M + 1) |
| 30 | isobutyl | mass spectrum (m/e): 204.1 (M + 1) |

### Example 31

### 2-Isopropyl-thiazole-4-carboxylic acid methyl ester

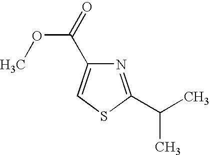

Treat 2-isopropyl-thiazolidine-4-carboxylic acid methyl ester (15.0 g, 79.24 mmol) with MnO₂ (103.3 g, 1188 mmol) in acetonitrile (300 ml) at room temperature for one hour, and heat to 70° C. for 4 hours. Cool the reaction mixture to room temperature, filter through a celite pad, concentrate filtrate under reduced pressure to give the title compound: mass spectrum (m/e): 185.9 (M+1).

Using the method of Example 31 gives the following compounds, isolated as the free base except where noted:

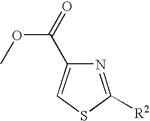

| NO.: | R² | DATA |
|---|---|---|
| 32 | isobutyl | mass spectrum (m/e): 213 (M + 1) |
| 33 | Tert-butyl | mass spectrum (m/e): 213.2 (M + 1) |

### Example 34

### 2-Isopropyl-thiazole-4-carboxylic amide

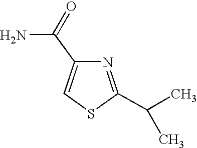

Treat 2-isopropyl-thiazole-4-carboxylic acid methyl ester (1 6.0 g, 86.3 mmol) with 7N NH₃ in methanol (800 ml) and stir the reaction for 18 hours at room temperature. Evaporate the reaction mixture under reduced pressure to give the title compound: mass spectrum (m/e): 171.2 (N+1).

Using the method of Example 34 gives the following compounds, isolated as the free base except where noted:

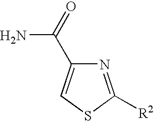

| No.: | R² | DATA |
|---|---|---|
| 35 | Tert-butyl | mass spectrum (m/e): 185.1 (M + 1) |
| 36 | isobutyl | mass spectrum (m/e): 184.9 (M + 1) |

### Example 37

### 2-Isopropyl-thiazole-4-carbonitrile

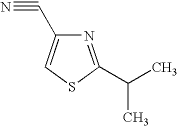

Add dropwise to a solution of 2-isopropyl-thiazole-4-carboxylic acid amide (1.0 g, 5.9 mmole) in dichloromethane (25 ml); POCl₃ (1.09 g, 7.08 mmol) in pyridine (3.0 ml) and stir the reaction mixture at room temperature. After 4 hours, treat the reaction mixture with brine, and evaporate under reduced pressure to give the title compound: mass spectrum (m/e): 153.2 (M+1).

Using the method of Example 37 gives the following compounds, isolated as the free base except where noted:

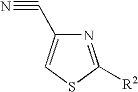

| NO.: | R² | DATA |
|---|---|---|
| 38 | isobutyl | mass spectrum (m/e): 167.2 (M + 1). |
| 39 | Tert-butyl | mass spectrum (m/e): 167.1 (M + 1). |

### Example 40

### 5-[Hydroxy-(2-nitro-phenyl)-methyl]-2-isopropyl-thiazole-4-carbonitrile

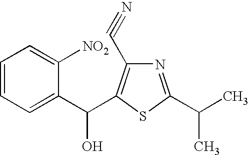

Add dropwise to a solution of 2-isopropyl-thiazole-4-carbonitrile (0.61 g, 4.0 mmol) in THF (20 ml) at −78° C., n-butyl lithium (3.12 ml, 5.0 mmole) and stir the reaction mixture at −78° C. After 1 hour, add dropwise 2-nitro-benzaldehyde in THF (5.0 ml). Allow the resulting solution to stir at −78° C. for an additional 1 hour, and warm to room temperature. Stir the reaction at room temperature overnight. Evaporate the reaction mixture under reduced pressure. Purification by flash chromatography, eluting with 10-20% ethyl acetate to give the title compound: mass spectrum (m/e): 303.7 (M+1).

Using the method of Example 40 gives the following compounds, isolated as the free base except where noted:

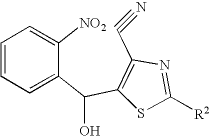

| NO.: | R² | DATA |
|---|---|---|
| 41 | isobutyl | Mass spectrum (m/e): 318.1 |
| 42 | Tert-butyl | Mass spectrum (m/e): 317.8 |

### Example 43

### 2-Isopropyl-5-(2-nitro-benzoyl)-thiazole-4-carbonitrile

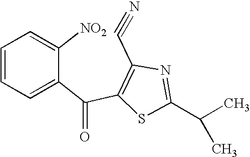

Add to a solution of 5-[hydroxy-(2-nitro-phenyl)-methyl]-2-isopropyl-thiazole-4-carbonitrile (2.2 g, 7.25 mmole) in dichloromethane (50 ml), pyridinium dichloromate (4;1 g, 10.88 mmole) and allow the reaction mixture to stir at room temperature. After 18 hours, filter the reaction mixture, evaporate the filtration under reduced pressure. Purification by flash chromatography, eluting with 20% ethyl acetate to give the title compound: mass spectrum (m/e): 302.1 (M+1).

Using the method of Example 43 gives the following compounds; isolated as the free base except where noted:

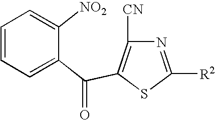

| NO.: | R² | DATA |
|---|---|---|
| 44 | isobutyl | Mass spectrum (m/e): 316.1 |
| 45 | Tert-butyl | Mass spectrum (m/e): 316.1 |

### Example 46

### 10-Amino-2-isopropyl-3-thia-1,9-diaza-benzo[f]azulen-4-one

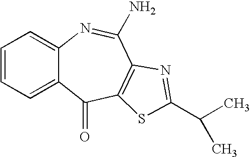

Add to a solution of 2-isopropyl-5-(2-nitro-benzoyl)-thiazole-4-carbonitrile (1.42 g, 4.63 mmol) in 4N HCl/dioxane (20 ml) at room temperature, SnCl₂ (2.63 g, 13.89 mmol) and stir the reaction mixture at room temperature. After 48 hours, treat the reaction mixture with 5N HCl (20 ml), stir for 30 min. and filter. Wash the solid with 5N HCl (20 ml), dry in oven to give the title compound: mass spectrum (m/e): 272.1 (M+1).

Using the method of Example 46 gives the following compounds, isolated as the free base except where noted:

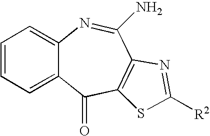

| NO.: | R² | DATA |
|---|---|---|
| 47 | isobutyl | Mass spectrum (m/e): 286.1 |
| 48 | Tert-butyl | Mass spectrum (m/e): 286.1 |

### Example 49

### 2-Isopropyl-4H-3-thia-1,9-diaza-benzo[f]azulen-10-ylamine

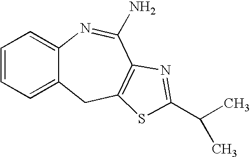

Add to a solution of 10-amino-2-isopropyl-3-thia-1,9-diaza-benzo[f]azulen-4-one (0.63 g, 2.4 mmole) in acetic acid (20 ml) at room temperature, zinc powder (1.56 g, 24.0 mmole). Heat the resulting mixture to 115° C. for 3 hours. Filter the reaction mixture, filtrate evaporate under reduced pressure to give the title compound: mass spectrum (m/e): 258.3 (M+1).

Using the method of Example 49 gives the following compounds, isolated as the free base except where noted:

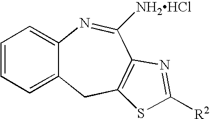

| NO.: | R² | DATA |
|---|---|---|
| 50 | Isobutyl | Mass spectrum (m/e): 272.1 |
| 51 | Tert-butyl | Mass spectrum (m/e): 272.0 |

### Example 53

### 2-Methyl-3,4-dithia-9-aza-benzo[f]azulen-10-ylamine

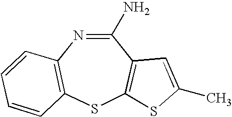

Stir sodium hydride (5.99 g, 60% dispersion, 150 mmol) in anhydrous tetrahydrofuran (200 ml). Dissolve 2-(2-Amino-phenylsulfanyl)-5-methyl-thiophene-3-carbonitrile (24.56 g, 99.7 mmol) in anhydrous tetrahydrofuran (300 ml). Heat mixture to 70° C. overnight then cool to room temperature. Quench reaction by addition of saturated sodium chloride solution then separate organic layer. Extract aqueous layer with ethyl acetate three times. Combine organic layers, dry over magnesium sulfate, filter then remove solvent by evaporation under reduced pressure. Purified by flash chromatography on silica. Gives 19.44 g, 79%. Mass spectrum 247 M+H for free base.

### Example 54

### 2-Bromo-5-ethylthiophene-3-carboxylic acid, methyl ester

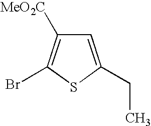

Add to a stirred suspension of copper(II)bromide (2.76 g, 12.4 mmol) in anhydrous acetonitrile (20mL), in an ice-bath at 0-5° C., t-butyl nitrite (1.56, 15 mmol) followed by 2-amino-5-ethylthiophene-3-carboxylic acid, ethyl ester (1.85 g, 10 mmol), portionwise, keeping the temperature at 0-3° C. The mixture was stirred in the ice-bath for 2 h then allowed to warm to ambient over 20 h, partitioned between EtOAc and dilute HCl, washed with saline solution and dried over anhydrous MgSO₄. The solvent was removed under reduced pressure and the residue was purified by flash chromatography on silica gel, eluting with 4-10% EtOAc in cyclohexane to give 1.28 g of the title compound, 95% pure by gc-ms (N+1; 250) 51% yield. ¹H-NMR (ppm): 1.1-1.35 (t, 3H); 2.65-2.88 (m, 2H), 3.72-3.9 (s, 3H).

### Example 55

### 2-Bromo-5-isopropylthiophene-3-carboxylic acid ethyl ester

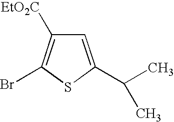

Using method similar to the method of 54, using 2-amino-5-isopropyl-thiophene-3-carboxylic acid, ethyl ester gives the title compound, 67% yield: ¹H-NMR (ppm): 1.25-1.35 (d, 6H) , (CH₃)CH; 1.35-1.45 (q, 3H, CH₃CH₂), 2.95-3.15 (m, 1H), CH(CH₃)₂; 4.3-4.4 (m, 2H, CH₂CH₃), 7.05(1H, H-4).

### Example 56

### 2-(2-Aminophenylsulfanyl)-5-ethylthionhene-3-carboxylic acid, methyl ester

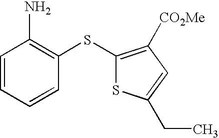

Stir methyl 2-bromo-5-ethylthiophene-3-carboxylate (7.84 g; 31.5 mmol), 2-aminothiophenol (3.675 mL, 34.3 mmol) and anhydrous cesium carbonate (I 1.25 g; 34.5 mmol) under a nitrogen atmosphere in dry DMF (150 mL) at 60° C. for 20 hours. Pour the mixture onto ice, extract into ethyl acetate, wash with 2 M HCl, saline, dry and evaporate the solvent. Purify the residue by flash chromatography on silica gel eluting with 25-33% EtOAc in cyclohexane to give 5.74 g of the title compound 93% pure by lc-ms (M+1; 294). 62% yield. ¹H-NMR (ppm): 1.12-1.2 (t, 3H); 2.55-2.7 (dd, 2H), 3.9 (s, 3H), 4.38-4.5 (br, 2H), 6.74-6.86 (m, 2H), 7.08, (s, 1H), 7.3-7.5 (m,1H), 7.45-7.55 (d,1H).

### Example 57

### 2-(2-Aminophenylsulfanyl)-5-isopropylthiophene-3-carboxylic acid, ethvl ester

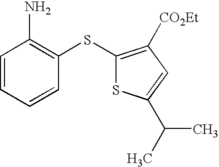

Using a method similar to Example 56, using 2-bromo-5-isopropyl-thiophene-3-carboxylic acid, ethyl ester gives the title compound 88% yield. m.s m/e (M+1; 322) ¹H-NMR (ppm): 1.18-1.25 (d, 6H), 1.38-1.48 (t, 3H), 2.85-3.0 (1H, sept), 3.9 (s, 3H), 4.31-4.61 (m, 3H), 6.74-6.84 (m, 2H), 7.05, (s, 1H,) 7.25-7.35 (m, 1H) 7.50-7.55 (d, 1H).

### Example 58

### 2-Ethyl-9H-3,4-dithia-9-azabenzo[f]azulen-10-one

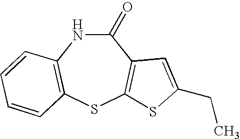

Add 2-(2-aminophenylsulfanyl-5-ethylthiophene-3-carboxylic acid, methyl ester (5.74 g, 19.6 mmol) in dry DMF (25mL) to a slurry of sodium hydride (1.17 g, 60% oil dispersion, 34.2 mmol) in dry DMF (5 mL) in an ice-bath at 5° C., slowly over 1.5 hours. Stir the mixture at 5° C. for 1 hour, pour onto ice and extract into EtOAc. Wash the extract with saline, dry over MgSO₄ and remove the solvent. Stir the crude product with EtOAc and filter to give the title compound 1.506 g, 30% (ms, m/e M+1: 262), ¹H-NMR (ppm): 1.18-1.32 (dd, 3H); 2.68-2.8 (q, 2H), 7.06-7.2 (m, 3H), 7.2-7.38 (m, 1H), 7.45-7.58 (dd, 1H), 7.9-8.05 (br, 1H).

### Example 59

### 2-Isopropyl-9H-3,4-dithia-9-azabenzo[f]azulen-10-one

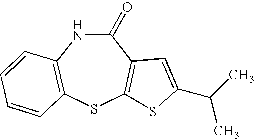

Heat 2-(2-aminophenylsulfanyl-5-isopropylthiophene-3-carboxylic acid, ethyl ester (7.34 g, 22.9 mmol) and ρ-toluenesulfonic acid monohydrate (800 mg, 4.2 mmol) under reflux in toluene (150 mL) for 20 hours. Cool the mixture and filter the precipitate, wash with toluene and dry to give the title compound: 3.013 g, 48% yield, pure by lc-ms (M+1; 276)

### Example 60

### 2-Isopropyl-9H-3,4-dithia-9-azabenzo[f]azulene-10-thione

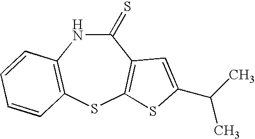

Heat 2-Isopropyl-9H-3,4-dithia-9-azabenzo[f]azulen-10-one (3.013 g, 10.95 mmol) and 2,4-bis(4-methoxyphenyl)-1,3-dithia-2,4-diphosphetane-2,4-disulfide (Lawesson's reagent) (2.66 g, 6.58 mmol) under reflux in toluene (50 mL) for 2 hours. Allow the yellow solution to cool to ambient, filter the precipitate and wash with toluene (2 mL) to give the title compound 2.74 g; 86% yield, m.p. 210° C. ¹H-NMR (ppm): 1.22-1.32 (d, 3H), 1.55-1.65 (br, 1H), 3.0-3.12 (sept, 1H) 7.16-7.42 (m, 5H).

### Example 61

### 2-Ethyl-9H-3,4-dithia-9-azabenzo[f]azulene-10-thione

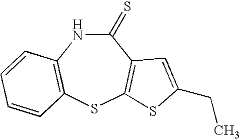

By using a method similar to Example 60, using 2-ethyl-9-H-3,4-dithia-9-azabenzo[f]azulen-10-one, Purification by flash chromatography on silica gel eluting with 20-100% EtOAc in cyclohexane to give the product in quantitative yield, sufficiently pure for the following reactions. ms m/e (M+1; 278), ¹H-NMR (ppm): 1.22-1.36 (dd 3H), 2.65-2.8 (q 2H), 7.18-7.52 (m 5H), 10.18-10.28 (br 1H).

### Example 62

### 4-Methyl pentanal

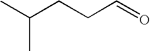

Dissolve 4-methyl pentanol (6.1 ml, 48.9 mmol) in methylene chloride (150 ml). Add pyridinium chlorochromate (15.88 g) and stir under nitrogen for 2 hours. Remove solvent by evaporation under reduced pressure at room temperature. Filter through Florisil™ and wash through with methylene chloride. Remove solvent by evaporation under reduced pressure at room temperature gives the title compound as colourless oil (3.1 g, 63%): ¹H NMR (CDCl₃): 0.9 (m, 6H, 2×CH₃), 1.5 (m, 3H, CH+CH₂), 2.4 (m, 2H, CH₂), 9.7 (m, 1H, CHO).

### Example 63

### 2-Amino-5-isobutyl-thiophene-3-carboxylic acid ethyl ester

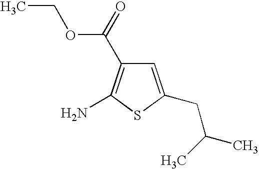

Add ethylcyanoacetate (3.5 ml), sulfur (1.02 g) and triethylamine (3 ml) to ethanol. Stir at 45° C. and add 4-methyl pentanal (3.1 g, 31 mmol) as a solution in N,N-dimethylformamide (30 ml). Stir at 45° C. for 2 hours then at room temperature overnight. Add water, extract into ethyl acetate. Separate layers and dry organic layer with magnesium sulfate, remove by filtration and remove solvent by evaporation under reduced pressure to the title compound 3.59 g of product, 51%. Mass spectrum, 227 M+H for free base.

### Example 64

### 2-Bromo-5-isobutyl-thiophene-3-carboxylic acid ethyl ester

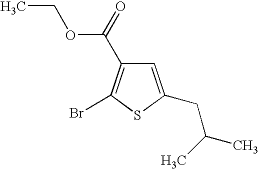

By using a method similar to the Example 55 gives the title compound: Mass spectrum 291/293 M+H.

### Example 65

### 2-(2-Amino-phenylsulfanyl)-5-isobutyl-thiophene-3-carboxylic acid ethyl ester

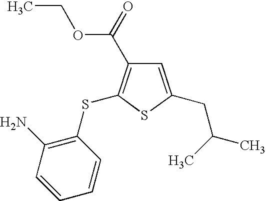

By using a method similar to the Example 56 gives the title compound. Mass spectrum 336 M+H for free base.

### Example 66

### 2-Isobutyl-9H-3,4-dithia-9-aza-benzo[f]azulen-10-one

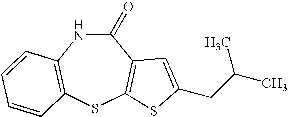

By using a method similar to the Example 58 gives the title compound: Mass spectrum 290 M+H.

### Example 67

### 2-Isobutyl-9H-3,4-dithia-9-aza-benzo[f]azulene-10-thione

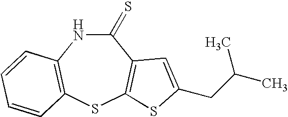

By using a method similar to the Example 58 gives the title compound: Mass spectrum 306 M+H.

### Example 68

### 2-Propyl-9H-3,4-dithia-9-aza-benzo[f]azulene-10-thione

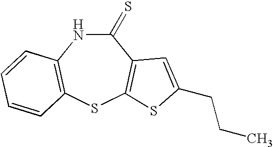

By using a method similar to the Example 58 gives the title compound:

### Example 69

### 2-(Amino-phenylsulfanyl)-5-bromo-thiophene-3-carboxylic acid ethyl ester

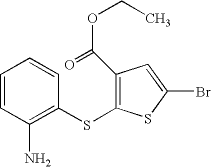

Dissolve 2,5-dibromo-thiophene-3-carboxylic acid ethyl ester (J. Org. Chem. 65(2000) 4618) (3.3 g, 10.5 mmol) in DMF (35 ml), add 2-aminothiophenol (135 g, 10.8 mmol) and cesium carbonate (4.5 g, 13.8 mmol). Stir the reaction mixture under nitrogen and heat to 70° C. in an oil bath overnight. TLC (cyclohexane/ethyl acetate 3:1) shows no starting material remains. Pour the reaction mixture into water and extract with ethyl acetate. Dry the organic phase (MgSO₄), filter and concentrate under reduced pressure to a dark oil. Column chromatography on silica, (eluent, cyclohexane/ethyl acetate 3:1) gives the desired title compound as an orange oil, 2.7 g, 72% yield. FIA mass spectroscopy shows a pair of peaks at 358/360 (MH+). ¹H NMR (CDCl₃) δ1.33 (q, 3H), 4.33 (t, 2H), 4.4 (broad, 2H), 6.8 (m, 2H), 7.29 (t, 1H), 7.48 (d, 1H).

### Example 70

### 2-Bromo-9H-3,4-dithia-9-aza-benzo[f]azulen-10-one

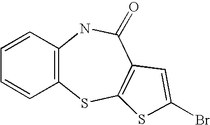

Dissolve 2-(amino-phenylsulfanyl)-5-bromo-thiophene-3-carboxylic acid ethyl ester (2.7 g, 7.54 mmol) in toluene (125 ml), and add p-toluenesulfonic acid, (140 mg, 0.74 mmol) and heat the reaction mixed under Dean-Stark conditions for 3 days. Allow the reaction mixture to cool and a white solid precipitates. Collect solid by filtration to give the desired title compound, 1.45 g, 62%. FIA mass spectroscopy shows a pair of peaks at 312/314 (NH+). ¹H NMR (d6 DMSO) δ7.2 (m, 1H), 7.24 (d, 1H), 7.33 (s, 1H), 7.42 (m, 1H), 7.53 (d, 1H), 10.6 (s, 1H).

### Example 71

### 2-Bromo-9H-3,4-dithia-9-aza-benzo[f]azulen-10-thione

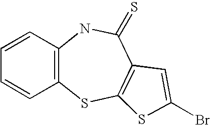

Suspend 2-bromo-9H-3,4-dithia-9-aza-benzo[f]azulen-10-one (1.4 g, 4.5 mmol) in toluene (40 ml) and add Lawesson's reagent (1.1 g, 2.72 mmol) and heat the mixture to reflux under nitrogen overnight. Allow mixture to cool and a yellow solid precipitates. Collect by filtration to give the desired title compound 1.4 g, 95%. FIA mass spectroscopy shows a pair of peaks at 328/330 (MH+).

### Example 72

### N-(2-nitro-phenyl)-thiourea-N′-carbamic acid ethyl ester

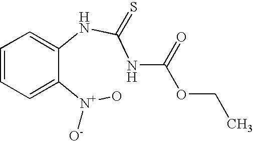

Add ethylisothiocyanatoformate (6.5 g, 50 mmol) to 2-nitroaniline (6.9 g, 50 mmol) in DMF (80 mL) at room temperature. Stir for one hour at room temperature then add ice. Filter and recrystallize from ethanol to yield the title compound as a yellow solid (10.1 g, 76 %): mp 117-120° C.; ¹HNMR (CDCl₃) δ 1.28 (t, 3H), 4.35 (q, 2H), 7.40 (t, 1H), 7.67 (t, 1H), 8.10 (d, 1H), 8.32 (bs, 1H), 8.44 (d, 1H); MS (APCI) m/z (rel intensity) 205 (100).

### Example 73

### 2-Isocyanato 1-phenylsulfanyl-benzene

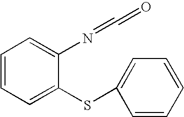 Add dropwise triphosgene (5 g, 16.7 mmol) in dioxane (50 mL) to 2-phenylsulfanyl-phenylamine (10 g, 50 mmol) in dioxane (100 mL) at ambient temperature and stir at 60° C. for 1 h. Concentrate and distill under high vaccum (bp=148-149° C./0.1 mm Hg) to give 7.5 g (67 %) of the title compound as a colorless liquid: ¹H NMR (DMSO-d₆) δ 7.37-7.10 (m, 8H), 7.42 (d, 1H); IR (nujol) 3170, 1648, 1318.

### Example 74

### 10H-Dibenzo[b,f][1,4]thiazepin-11-one

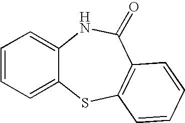 Add dropwise 2-isocyanato-1-phenylsulfanyl-benzene (7.2 g, 31 mmol) in o-dichlorobenzene (32 mL) to Aluminium trichloride (4.2 g, 31 mmol) in o-dichlorobenzene (32 mL) at 100° C., stir at 150° C. for 4 h and cool down to ambient temperature. Concentrate and add ice to the residue. Filter the resulting solid, dry to give 7.1 g (quant) of the title compound as a beige solid: ¹H NMR (DMSO-d₆) δ 7.11 (dt, 1H), 7.20 (dd, 1H), 7.32 (dt, 1H), 7.54-7.37 (m, 4H), 7.65 (dd, 1H); IR (nujol): 3170, 1698, 1376.

### Example 75

### 11-Chloro-dibenzo[b,f][1,4]thiazepine

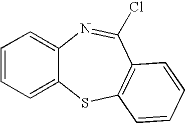

Combine 10H-Dibenzo[b,f][1,4]thiazepin-11-one (3 g, 13 mmol), phosphorus oxychloride (12 g, 80 nunol), dimethyl aniline (0.52 g, 4 mmol), reflux for 4 h, cool to ambient temperature and concentrate. Dilute in toluene (100 mL), add ice, wash with water and brine and dry over magnesium sulfate. Evaporate to give 2.9 g (90%) of the title compound as a tan solid: mp=93-97° C.; ¹H NMR (CDCl₃) δ 7.17 (ddd, 1H), 7.47-7.26 (m, 6H), 7.17 (ddd, 1H); MS (APCI) m/z (rel intensity) 246.3 (100).

### Example 77

### 2-Ethyl-4,5-dihydro-thiazole-4-carboxylic acid ethyl ester

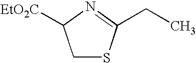

Prepared according to Tetrahedron: Asymmetry 12 (2001) 1279-1286

### Example 78

### 2-Isopropyl-4,5-dihydro-thiazole-4-carboxylic acid ethyl ester

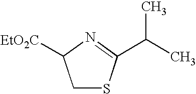

Prepared according to Tetrahedron: Asymmetry 12 (2001) 1279-1286

### Example 79

### 2-Ethyl-thiazole-4-carboxylic acid ethyl ester

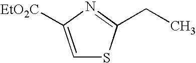

Dissolve 2-ethyl-4,5-dihydro-thiazole-4-carboxylic acid ethyl ester (12.3 g, 66 mmol) in dichoromethane with stirring and cool reaction mixture in an ice-bath. Add 1,8-diazabicyclo[5.4.0]undec-7-ene (11.5 g, 75 mmol) in one portion. Then add bromotrichloromethane (15 g, 75 mmol) dropwise. The resulting mixture is stirred to room temperature and stirring continued overnight. Wash the solution with 1 M HCl solution, dry over sodium sulfate, and concentrate under reduced pressure to give a dark oil. Purification of the residue by flash chromatography eluting with ethyl acetate:cyclohexane (1:6 to 1:2 gradient), gives the title compound as a yellow oil (10 g,): Mass Spectrum (m/e): 186 (M+1).

### Example 80

### 2-Isopropyl-thiazole-4-carboxylic acid ethyl ester

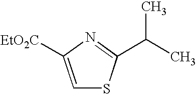

By using a method similar to Example 79, the title compound 2-isopropyl-4,5-dihydro-thiazole-4-carboxylic acid ethyl ester, is prepared. Mass Spectrum (m/e): 200 (M+1).

### Example 81

### Ethyl 2-aminothiazole-4-carboxylate hydrobromide salt

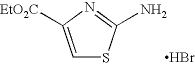

Prepared according to Tetrahedron: Asymnmetry 9 (1998) 1395-1407

### Example 82

### 5-Bromo-2-ethyl-thiazole-4-carboxylic acid ethyl ester

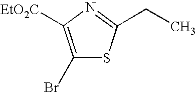

Add 2-ethyl-thiazole-4-carboxylic acid ethyl ester (7 g, 38 mmol) in dry THF (80 mL) dropwise to a cold (−78° C.) solution of LDA in dry THF (80 mL) under a nitrogen atmosphere with stirring. Stiring continued for 40 min. Add a solution of dibromotetrachloroethane in dry THF (30 mL) dropwise at −78° C. The resultng mixture is stirred to room temperature and stirring continued overnight. Quench the reaction by addition of aqueous ammonium chloride solution. Partially evaporate the solvent and extract the product into ethyl acetate. Wash the solution with 1 M HCl_{(aq)}, dry over sodium sulfate, and concentrate under reduced pressure to give a dark oil. Purification of the residue by flash chromatography eluting with ethyl acetate:cyclohexane (1:1), gives the title compound as a yellow oil (2 g): Mass Spectrum (m/e): 264/266 (M+1).

### Example 83

### 5-Bromo-2-isopropyl-thiazole-4-carboxylic acid ethyl ester

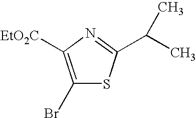

By using a method similar to Example 82, the title compound 5-bromo-2-isopropyl-thiazole-4-carboxylic acid ethyl ester, is prepared. Mass Spectrum (m/e): 278/280 (M+1).

### Example 84

### Ethyl 2-amino-5-chlorothiazole-4-carboxylate

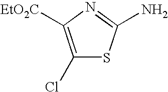

Prepared according to J. Heterocyclic Chem., 28 (1991) 1003-1011.

### Example 85

### Ethyl 5-chlorothiazole-4-carboxylate

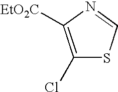

Dissolve ethyl 2-amino-5-chlorothiazole-4-carboxylate (1.0 g, 4.85 mmol) in DMF (6 mL) and heat to 65° C. Add dropwise a solution of t-butyl nitrite (0.633 mL) in DMF (10 mL) with stirring. Care: gas evolution. After the addition is complete, stir the reaction mixture for 10 min at 65° C. and then cool to room temperature. Pour into water and extract the product into diethylether (×2). Combine organic extracts and dry (MgSO₄) and concentrate to give the titled compound contaminated with trace DMF (590 mg). Mass Spectrum (m/e): 192/194 (N+1).

### Example 86

### N-(5-Chloro-4-dichloromethyl-thiazol-2-yl)-acetamide

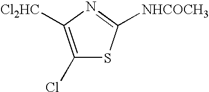

Based on method from EP 0160818.

Dissolve a mixture of 2-acetamido-4-methyl thiazole (3,42 g, 20 mmol) and N-chlorosuccinimide (8.8 g, 66 mmol) in DMF (10 ml), heat at 70° C. overnight. Pour into ice water and collect the buff coloured solid by filtration. Wash well with water and dry in vacuo at room temperature (5.13 g). Mass spectrum (m/e) 259, 261, 263, 265 (M+1).

### Example 87

### N-[5-Chloro-4(hdroxyimino-methyl)-thiazol-2-yl]-acetamide

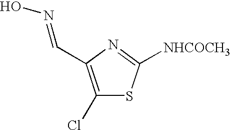

Take a mixture of N-(5-chloro-4-dichloromethyl-thiazol-2-yl)-acetamide (5.1 g, 20 mmol), sodium formate (3.3 g, 48 mmol) and hydroxylamine hydrochloride (1.66 g, 24 mmol) in formic acid (30 ml) and heat at 100° C. for 1 hour. Pour into ice water and collect the white solid by filtration. Well wash with water and dry in vacuo at 40° C. (3.13 g). Mass spectrum (m/e) 220-222 (M+1).

### Example 88

### N-(5-Chloro-4-cyano-thiazol-2-yl)-acetamide

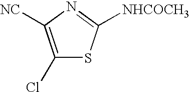

Mix N-[5-chloro-4(hydroxyimino-methyl)-thiazol-2-yl]-acetamide (3:1 g, 15 mmol) and acetic anhydride (30 ml) and heat at reflux for 2 days. Pour into ice water, extract with ethyl acetate, well wash with water, dry (MgSO₄) and concentrate to leave a white solid (2.25 g). Mass spectrum (mn/e) 202-204 (M+1).

### Example 89

### N-[5-(2-Amino-phenylsulfanyl)-4-cyano-thiazol-2-yl)-acetamide

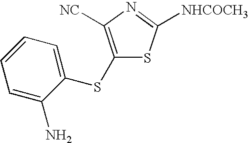

Take a nixture of N-(5-chloro-4-cyano-thiazol-2-yl)-acetamide (2.2 g, 11 mmol), o-aminothiophenol (1.51 g, 12 mmol) and cesium carbonate (3.91 g, 12 mmol) in DMF (20 ml) and heat at 70° C. overnight. Pour the reaction mixture into ice water and extract with ethyl acetate, wash well with brine, dry (MgSO₄) and concentrate to give the title product as a white solid (2.6 g). Mass spectrum (m/e) 291 (M+1).

### Example 90

### 5-(2-Amino-phenylsulfanyl)-2-ethyl-thiazole-4-carboxylic acid ethyl ester

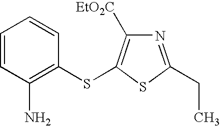

Dissolve 5-bromo-2-ethyl-thiazole-4-carboxylic acid ethyl ester (2 g, 7.5 mmol) in DMF (20 ml) and add 2-aminothiophenol (1.15 g, 9.2 mmol) and cesium carbonate (3.3 g, 10.1 mmol), stir the reaction mixture at 78° C. overnight. Treat the reaction mixture with dilute NaOH solution and extract the product into ethyl acetate. Separate the organic layer, wash with brine and dry (MgSO₄) and concentrate to give the titled compound as a dark oil (2.25 g). This was used without purification. Mass spectrum (m/e): 309 (M+1).

### Example 91

### 5-(2-Amino-phenylsulfanyl)-2-isopropyl-thiazole-4-carboxylic acid ethyl ester

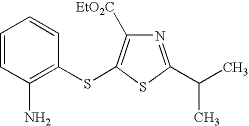

By using a method similar to Example 90, the title compound 5-(2-amino-phenylsulfanyl)-2-isopropyl-thiazole-4-carboxylic acid ethyl ester, is prepared. Mass spectrum (m/e): 323 (M+1).

### Example 92

### 5-(2-Amino-phenylsulfanyl)-2-thiazole-4-carboxylic acid ethyl ester

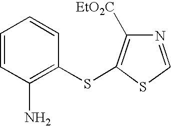

By using a method similar to Example 90, the title compound 5-(2-amino-phenylsulfanyl)-2-thiazole-4-carboxylic acid ethyl ester, is prepared. Mass spectrum (m/e): 281 (M+1).

### Example 93

### 2-Ethyl-9H-3,4-dithia-benzo[f]azulen-10-one

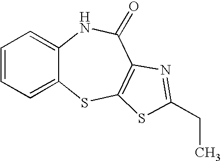

Dissolve 5-(2-amino-phenylsulfanyl)-2-ethyl-thiazole-4-carboxylic acid ethyl ester (2.25 g, 7.3 mmol) in toluene with stirring and add para-toluene sulfonic acid (100 mg). Heat the resulting solution at reflux under Dean and Stark conditions over 3 days. Cool reaction to room temperature and concentrate to give a dark oil. Purification by flash chromatography eluting with ethyl acetate:cyclohexane (1:1), gives the title compound as a oily solid (1.2 g): Mass Spectrum (m/e): 263 (M+1).

### Example 94

### 2-Isopropyl-9H-3,4-dithia-benzo[f]azulen-10-one

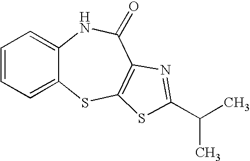

By using a method similar to Example 93 and 5-(2-amino-phenylsulfanyl)-2-isopropyl-thiazole-4-carboxylic acid ethyl ester, the title compound 2-isopropyl-9H-3,4-dithia-benzo[f]azulen-10-one, is prepared. Mass Spectrum (m/e): 277 (M+1).

### Example 95

### 9H-3,4-dithia-1,9-diaza-benzo[f]azulen-10-one

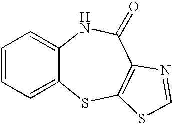

By using a method similar to Example 93 and 5-(2-amino-phenylsulfanyl)-2-thiazole-4-carboxylic acid ethyl ester, the title compound 9H-3,4-dithia-1,9-diaza-benzo[f]azulen-10-one, is prepared. Product is collected by fitration from the cooled reaction mixture. Mass Spectrum (m/e): 235 (M+1).

### Example 96

### N-(10-Amino-3,4-dithia-1,9-diaza-benzo[f]azulen-2-yl)-acetamide acetic acid salt

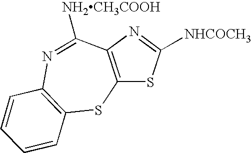

Dissolve N-[5-(2-Amino-phenylsulfanyl)-4-cyano-thiazol-2-yl)-acetamide (2.5 g, 8.6 mmol) in acetic acid (25 ml) and heat at reflux overnight. Pour the reaction mixture into ice water and collect the white solid by filtration, wash with water and dry in vacuo at 40° C. (2.6 g). Mass spectrum (m/e) 291 (M+1).

### Example 97

### 2-Ethyl-9H-3,4-dithia-benzo[f]azulen-10-thione

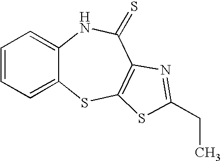

Slurry 2-ethyl-9H-3,4-dithia-benzo[f]azulen-10-one (1.2 g, 4.58 mmol) and Lawesson's reagent [2,4-bis(4-methoxyphenyl)-1,3-dithia-2,4-diphosphetane-2,4-disulfide] (1.2 g, 2.2 mmol) in toluene and heat to reflux under a nitrogen atmosphere for 30 min. Cool reaction mixture to ambient temperature, then concentrate under reduced pressure. Purification of the residue via flash chromatography, eluting with a ethyl acetate:cyclohexane (1:1) gives the title compound: Mass Spectrum (m/e): 279 (M+1).

### Example 98

### 2-Isopropyl-9H-3 4-dithia-benzo[f]azulen-10-thione

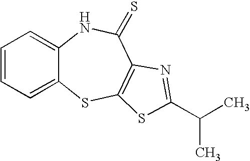

By using a method similar to Example 97 and 2-isopropyl-9H-3,4-dithia-benzo[f]azulen-10-one, the title compound 2-isopropyl-9H-3,4-dithia-benzo[f]azulen-10-thione, is prepared. Mass Spectrum (m/e): 293 (M+1).

### Example 99

### 9H-3,4-dithia-1,9-diaza-benzo[f]azulen-10-thione

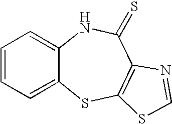

By using a method similar to Example 97 and 9H-3,4-dithia-1,9-diaza-benzo[f]azulen-10-one, the title compound 9H-3,4-dithia-1,9-diaza-benzo[f]azulen-10-thione, is prepared. Mass Spectrum (m/e): 251 (M+1).

### Example 100

### 3-(2-Methoxy-ethyl)-1-trityl-piperazine

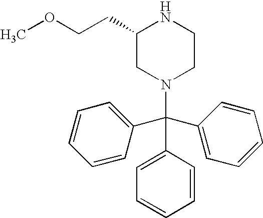

Combine 2-(2-methoxy-ethyl)-piperazine (1.95 g, 13.5 mmol), trityl chloride (3.77 g, 13.5 mmol) and triethyl amine (1.36 g, 13.5 mmol) and stir in dichloromethane (50 ml ) at room temperature for 3 hours. Wash the reaction mixture with brine and dry (MgSO₄). Evaporate under reduced pressure to give the title compound- mass spectrum (m/e): 387.3 (M+1).

### Example 101

### 2-(2-Methoxy-ethyl)-1-methyl-4-trityl-piperazine

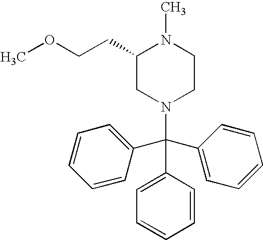

Combine 3-(2-methoxy-ethyl)-1-trityl-piperazine (5.0 g, 12.9 mmol) in dichloromethane (150 ml) at room temperature and add 38% aq. formaldehyde (2.6 g, 26.0 mmol) and sodium triacetoxyborohydride (4.1 g, 19.4 mmol) and stir. After 1 hour, pour the reaction mixture into a separatory funnel containing brine (200 ml). Dry the organic layer (MgSO₄) and evaporated under reduced pressure to give the title compound.

### Example 102

### 2-(2-Methoxy-ethyl)-1-methyl-piperazine

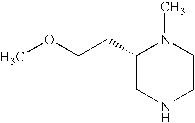

Dissolve 2-(2-methoxy-ethyl)-1-methyl-4-trityl-piperazine (2.5 g, 6.3 mmol) in ethanol (50 ml) at room temperature and add 1N HCl (3.0 ml) and stir at room temperature. After 1 hour, evaporate the reaction mixture under reduced pressure. Take up the residue with ethanol (1.0 ml), triturate with ether, filter the precipitate to give the title compound: mass spectrum (m/e): 159.2(M+1).

### Example 103

### (S)-1,4-Dibenzyl-2-vinylpiperazine

### Example 104(R)-1,4-Dibenzyl-2-vinylpiperazine

Add anhydrous tetrahydrofuran (4.5 L) to a 10 L flange-neck flask equipped with an air stirrer rod and paddle, thermometer, and nitrogen inlet and outlet tubes. Purge with dry nitrogen gas (inlet tube had a sintered end for maximum gas dispersal) the body of the liquid for 1 h, add tris(dibenzylideneacetone)dipalladium(0) chloroform adduct (36.0 g, 34.8 mmol). Add isopropyl phosphite (67.8 mL, 0.275 mol) in one lot to the mixture still under nitrogen and stir. After 5 minutes, the color lightens from purple to amber. Add dibenzylethylenediamine (322.0 g, 1.34 mol) in one lot, followed by the dropwise addition of cis-1,4diacetoxy-2-butene (214 mL, 1.34 mol) over 15 minutes stir under nitrogen for 18 hours. Remove the solvent in vacuo at 40° C. and dissolve the residual oil in diethyl ether (2.5 L) and extract with 1N aq. sodium hydroxide (2×2 L). Wash the bulked aqueous extracts with diethyl ether (2×) and basify to pH 14 using 5N aq. sodium hydroxide and extract with diethyl ether (3×). Dry the bulked ethereal extracts over magnesium sulphate, filter and evaporate to dryness in vacuo at 40° C. Purification by chromatography on silica (1.17 kg) using 1% methanol/ether (can also use dichloromethane) gives a pale yellow oil (377.35 g, 96%) 1H NMR and Mass Spec are consistent with product.

Dissolve the mixture of isomers in ethyl acetate (3670 mL) and add portionwise to a hot solution of (S)-(+)-mandelic acid (385 g, 2 eq.) in ethyl acetate (3850 mL), starting at 72° C. Chill the mixture to 0° C. and seed with crystals (obtained from an earlier resolution). Place the mixture in the freezer (−20° C.) overnight. Scrape the crystalline solid away from the sides of the flask and allow the mixture to warm to 0° C. Isolate the solid dry. Further dry the material in vacuo at room temperature. Yield=252.6 g, white, crystalline solid of the S-mandelic acid salt of the (R)-1,4-dibenzyl-2-vinylpiperazine.

Evaporate the filtrate to dryness in vacuo at 40° C. to leave an amber oil. Dissolve the filtrate in dichloromethane (2 L) and wash the solution with 1N aq. sodium hydroxide (2 L+1 L), brine (1 L) and dry over magnesium sulphate. Filter and evaporate to dryness in vacuo at 45° C. to yield the recovered free base. Further dry by vacuum. Extract the aqueous liquors with dichloromethane to firther recover any remaining free base (207.6 g). Chiral HPLC showed the material to consist of a 85:15 ratio of isomers in favour of the required-isomer.

Add (R)-(−)-mandelic acid (216 g, 1.42 mol) and ethyl acetate (2.5 L) to a 10 liter flange-neck flask equipped with an air stirrer rod and paddle, thermometer and water condenser and warm the suspension to 60° C. Add a solution of free base (207.6 g, 0.71 mol) in ethyl acetate (500 mL) and allow to cool down to room temperature and place in the freezer overnight (at 35° C. solid starts to precipitate). Isolate the crystalline solid by filtration and pull dry. Further dry in vacuo at room temperature (290.34 g).

Recrystallize from hot ethyl acetate (2.3 L) at 70° C. Allow this solution to cool down to room temperature overnight after seeding. Filtration and drying in vacuo at room temperature gives the (R)-mandelic acid salt of the (S)-1,4-dibenzyl-2-vinylpiperazine from which the free base may be prepared (225.44 g). Chiral HPLC showed: 98.74%+1.26%: ¹H NMR, (DMSO-d₆): δ 7.20-7.35 (m, 10H); 5.75-5.90 (m, 1H); 5.15-5.30 (q, 2H); 3.85-3.95 (d, 1H); 3.40-3.45 (s, 2H); 3.00-3.10 (d, 1H); 2.80-2.90 (t, 1H); 2.55-2.60 (d, 3H); 1.95-2.10 (m, 3H).

### Example 105

### (5)-1,4-Dibenzyl-2-Vinyl-Piperazine

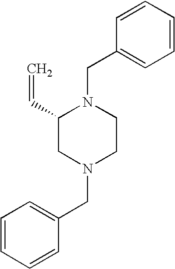

Combine (S)-1,4-dibenzyl-2-vinyl-piperazine mandelic acid salt (200.0 g, 0.450 mol), water (1 L), and 5N sodium hydroxide (112.5 mL, 0.562 mol) and stir at ambient temperature for 15 minutes. Add MTBE (1 L) and stir at ambient temperature for 1 hour. Layer separate and wash the organics with water (2×1 L). Dry the organics over sodium sulfate, filter and concentrate in vacuo to afford 131.2 g of the title compound as an oil (99.7%). ¹H NMR (500 MHz, DMSO-d₆) δ 2.00 (bt, 1H), 2.07 (d, 2H), 2.56 (m, 1H), 2.58 (d, 2H), 2.84 (t, 1H), 3.02(d, 1H), 3.49 (s, 2H), 3.91 (d, 1H), 5.15 (d, 1H), 5.25 (d, 1H), 5.81 (m, 1H), 7.27 (m, 10H). MS (ES+) M+H calcd for C₂₀H₂₄N₂ 292.43; found 293.10.

### Example 105a

### (S)-2-(1,4-Dibenzyl-piperazin-2-yl)-Ethanol

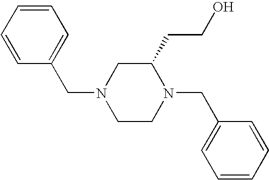

Combine (S)-1,4-dibenzyl-2-vinyl-piperazine (131.0 g, 0.448 mol) and THF (500 mL) at ambient temperature. Add a 0.5 M THF solution of 9-borabicyclo[3.3.1] nonane (9-BBN) (985.5 mL, 0.493 mol) over 20 minutes, keeping the pot temperature below 25° C. Stir the resulting mixture at ambient temperature for 16-18 hours. Cool the solution to 0-5° C. and quench with 3N NaOH (164.3 mL, 0.493 mol) over 5-10 minutes. Stir at 0-5° C. for 10-15 minutes and remove cooling bath. Add a 30% aqueous solution of hydrogen peroxide (160.2 mL, 1.57 mol) over 1 hour, maintaining a pot temperature of 30-35° C. Stir the resulting mixture for 1 hour and dilute with water (1.31 L) and MTBE (1.31 L). Layer separate and re-extract the aqueous layer with MTBE (655 mL). Combine organics and wash with water (2×655 mL) and brine (2×655 mL). Dry the MTBE solution over sodium sulfate, filter and concentrate in vacuo to afford 169.1 g of crude oil (theory=139.1 g). Treat the crude with 5N HCl (358 mL, 1.79 moles) and stir 5 minutes. Add water (250 mL) and stir 15-20 minutes. Add heptane (1.31 L) and stir 5 minutes. Layer separate and re-extract the aqueous layer with MTBE (655 mL). Discard these organic extracts and add 5N NaOH (403 mL, 2.02 moles) to the aqueous portion. Stir 5 minutes then dilute with MTBE (1.31 L) and layer separate. Re-extract the aqueous layer with MTBE (655 mL). Combine the MTBE extracts. and wash with water (655 mL), then dry over sodium sulfate, filter and concentrate in vacuo to afford 137.8 g (99.0%) of the title compound. ¹H NMR (500 MHz, DMSO-d₆) δ 1.87 (m, 1H), 2.03 (m, 1H), 2.33 (m, 2H), 2.43 (m, 1H), 2.50 (d, 1H), 2.66 (d, 1H), 2.83 (m, 1H), 2.93 (m, 1H), 3.39 (d, 1H), 3.50 (m, 1H), 3.74 (m, 1H), 3.88 (m, 1H), 4.18 (d, 1H), 4.80 (bs, 1H), 7.26 (m,2H), 7.32 (m, 8H).

### Example 105c

### (S)-1,4-Dibenzyl-2-(2-methoxy-ethyl)-piperazine

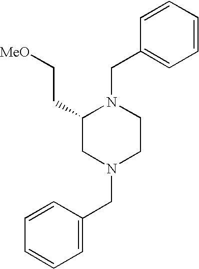

Combine sodium hydride (52.9 g, 1.32 moles) and THF (500 mL) and cool the resulting slurry to 0-5° C. Dissolve (S)-2-(1,4-dibenzyl-piperazin-2-yl)-ethanol (137.0 g, 0.441 mol) in THF (500 mL) and add to the sodium hydride/THF mixture over 30 minutes, maintaining a pot temperature of 0-10° C. Stir the mixture at 0-10° C. for 15-20 minutes, then add dimethyl sulfate (55.6 g, 0.441 mol) over 1 hour, maintaining a pot temperature of 0-10° C. Remove cooling bath and allow reaction mixture to warm to ambient temperature over 1 hour. Stir an additional hour at ambient temperature, re-cool to 0-10° C., then quench with 1N ammonium chloride (863 mL). Extract the mixture with ethyl acetate (2×700 mL). Combine the ethyl acetate extracts and wash with saturated aqueous sodium bicarbonate (2×2 L), dry over magnesium sulfate and concentrate in vacuo to afford 162.1 g of oil. Dissolve the oil in heptane (900 mL) and add water (450 mL). While stirring, add 3N HCl to a pH of 1-2. Layer separate and treat the aqueous layer with 50% caustic to a pH of 13-14. Extract this aqueous solution with methylene chloride (2×1 L), combine the organic extracts and dry over magnesium sulfate. Concentrate in vacuo to afford 136.3 g (93.3%) of the title compound. ¹H NMR (400 MHz, DMSO-d₆) δ 1.80 (m, 2H), 2.20 (bm, 2H, 1H), 2.41 (m, 1H), 2.55 (m, 2H), 2.60 (d, 1H), 3.18 (s, 3H), 3.35 (bm, 2H, 1H), 2.40 (d, 1H), 2,52 (d, 1H), 3.90 (d, 1H), MS (ES+) calcd for C₂₁H₂₈N₂ 324.47; found 325.2.

### Example 105d

### (S)-2-(2-Methoxy-ethyl)-piperazine

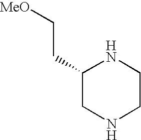

Dissolve (S)-1,4-dibenzyl-2-(2-methoxy-ethyl)-piperazine (2.0 g, 6.2 mmol) in ethanol (15 mL) in a suitable hydrogenation vessel. Add palladium hydroxide (Pearlman's catalyst, 400 mg), purge vessel with nitrogen, then pressure to 60 psi. Heat to 50° C. and stir vigorously for 18-24 hours. Allow the mixture to cool to ambient temperature. Filter off the catalyst and rinse with ethanol (5 mL). Concentrate in vacuo using a 35° C. bath to afford the title compound (0.81 g, 91.1%). ¹H NMR (500 MHz, DMSO-d₆) δ 1.41 (m, 2H), 2.12 (t, 1H), 2.43 (td, 1H), 2.53 (m, 2H, 1H), 2.63 (d, 1H), 2.72 (m, 2H), 3.19. (s, 3H), 3.34 (m, 2H).

### Example 106

### (S)-1,4-Dibenzyl-2-(2-(3-fluoro-phenyl)-ethyl)-piperazine

Combine (S)-1,4-dibenzyl-2-vinyl-piperazine (2.0 g, 6.84 mmol) and 9-borabicyclo[3.3.1]nonane (82.1 mL, 41.04 mmol, 0.5 M in THF) and stir at ambient temperature. After 24 hours, add 1-fluoro-3-iodo-benzene (2.3 g, 10.26 mmol), triphenylphosphine (287.0 mg, 1.09 mmol), tetrakis(triphenylphosphine) palladium(0)(158.0 mg, 0.14 mmol), and 3N NaOH (5.6 mL) and stir at 60° C. After 22 hours, add ethanolamine (10.0 mL) and dilute the mixture with water. Extract with ethyl acetate and combine, wash (brine), dry (sodium sulfate), and reduce the extracts to residue. Purify the residue on silica gel using ethyl acetate/hexanes (5:95) to give a yellow oil. Dissolve the yellow oil in acetic acid/methanol (1:9) and apply to an SCX column. Wash the column with methanol followed by 2N ammonia in methanol to give the title compound: mp 69-71° C.; mass spectrum (ion spray): m/z=389.4 (M+1).

### Example 107

### (S)-2-(2-(3-Fluoro-phenyl)-ethyl)-piperazine

Combine (S)-1,4-dibenzyl-2-(2-(3-fluoro-phenyl)-ethyl)-piperazine (1.96 g, 5.04 mmol), ammonium formate (1.59 g, 25.18 mmol), 5% Pd/C (243.3 mg), and ethanol (50 mL). Stir and heat the mixture at reflux. After 4 hours 30 minutes, cool to ambient temperature and remove the catalyst by vacuum filtration through celite. Reduce the filtrate to residue and purify it on silica gel using dichloromethane/2N ammonia in methanol (80:20) to give the title compound as a white solid: mp 95-99° C.; mass spectrum (ion spray): m/z=209.3 (M+1); Analysis for C₁₂H₁₇FN₂: calcd: C, 69.20; H, 8.23; N, 13.45; found: C, 69.13; H, 8.40; N, 13.28.

### Example 108

### (S)-(4-Benzyl-3,6-dioxopiperazin-2-yl)acetic acid methyl ester

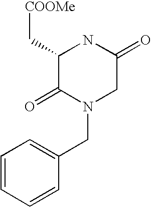

Dissolve commercial N-tBoc-L-aspartic acid β-methyl ester (40 g, 0.16 mol) in dichloromethane (800 mL); cool to 0° C. and add N-benzylglycine methyl ester (28 g, 0.15 mol added as a solution in 100 mL of dichloromethane), followed sequentially by N,N-diisopropylethylamine (28 mL, 0.16 mol), 1-ethyl-3-(3-dimethylaminopropyl) carbodiimide hydrochloride (EDAC, 31 g, 0.16 mol), and 1-hydroxybenzotriazole (22 g, 0.16 mol). Stir at room temperature over the weekend and concentrate in vacuo to an orange oil. Partition oil between 2N hydrochloric acid and ethyl acetate; separate aqueous layer and extract with a second portion of ethyl acetate. Combine organic extracts, concentrate in vacuo, and wash with 10% aqueous potassium carbonate. Dry organic layer over magnesium sulfate, filter and concentrate in vacuo to yield 64 g (95%) of the desired dipeptide as an oily residue.

Dissolve the crude dipeptide in 150 mL of trifluoroacetic acid, stir at room temperature for 1 h; then remove the solvent in vacuo. Take up the resulting residue on 800 mL of commercial 2N ammonia in methanol solution, and stir at room temperature overnight. Heat the mixture at 70° C. for several hours; then cool to room temperature and remove the solvent in vacuo. Redissolve the residue in dichloromethane, filter off the resulting precipitate, and concentrate the filtrate in vacuo. Apply the residue to a silica gel column. Elute with a 2% mixture of 2N ammonia-methanol in dichloromethane to obtain 31.9 g (72%) of the title compound as a yellow oil: mass spectrum (APCI): m/e 277.1 (M+1).

### Example 109

### (S)-(−)-2-(4-Benzylpiperazin-2-yl)ethanol

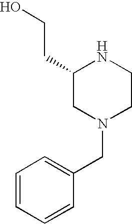

To a 0° C. solution of (S)-(4-benzyl-3,6-dioxopiperazin-2-yl)acetic acid methyl ester (31.9 g, 0.12 mol) in tetrahydrofuiran (1 L), add lithium aluminum hydride via slow cannulation (350 mL of a commercial 1.0 M solution in tetrahydrofuran). Stir at room temperature overnight, quench by successive careful addition of 13.3 mL of water, 13.3 mL of 15% aqueous sodium hydroxyde, and 39.9 mL of water, all the while with vigourous stirring to ensure formation of a fine precipitate. Filter through a fritted funnel, washing the solids well with tetrahydrofuran and dichloromethane. Concentrate in vacuo to provide 26.5 g of an oily residue, apply directly to a silica gel column. Elute with a 5% mixture of 7N ammonia-methanol in dichloromethane, to obtain the desired product as an orange oil which solidifies under vacuum. Take up the solid in acetonitrile and sonicate for a few minutes. Filter the resulting precipitate to obtain 7.5 g (25%) of the title compound as an off-white crystalline solid, mp 78.9-80.4° C. Concentrate the mother liquor to obtain 6.8 g (23%) of slightly less pure material as an amorphous solid: mass spectrum (ES): m/e 221.3 (M+1); specific Rotation: −7.84.

### Example 110

### (S)-4-Benzyl-2-(2-hydroxyethyl)piperazine-1-carboxylic acid tert-butyl ester

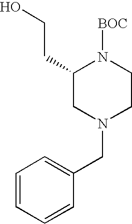

To a solution of (S)-(−)-2-(4-benzylpiperazin-2-yl)ethanol (14.9 g, 67.6 mmol) in dichloromethane (200 mL) add di-tert-butyl dicarbonate (15.5 g, 71 mmol) as a solution in dichloromethane (30 mL). Stir at room temperature 4 h, partition between saturated. aqueous bicarbonate and dichloromethane and extract aqueous layer with additional dichloromethane. Combine organic extracts, dry over sodium sulfate, filter and concentrate in vacuo to a residue. Apply residue to silica gel column, eluting with 5% 2N ammonia-methanol in dichloromethane, to obtain title compound as a yellow oil: mass spectrum (APCI): m/e 321.2 (M+1).

### Example 111

### (S)-2-(1,4-Dibenzylpiperazin-2-yl)ethanol

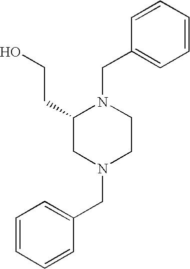

Dissolve (S)-1,4-dibenzyl-2-vinylpiperazine (16.2 g, 55.5 mmol) in tetrahydrofuran (370 mL), add 9-BBN (0.56 L of a 0.5 M solution in tetrahydrofuran) via addition funnel, stir at room temperature overnight. Cool to 0° C. and treat with 30% aqueous hydrogen peroxide (195 mL), followed by 3N aqueous sodium hydroxide (195 mL). Allow to reach room temperature and stir for 24 h. Pour into separatory funnel, separate organic layer and remove solvent in vacuo. Take up residue in dichloromethane, add water and recombine with aqueous layer from before. Pour into separatory funnel, separate organic layer and extract aqueous layer with several portions of dichloromethane. Combine all organic layers, remove solvent in vacuo. Take up residue in 1 L of methanol, add 185 g of SCX resin. Filter slurry through Büchner funnel, washing well with methanol. To elute product, wash cake thoroughly with 50% 7N ammonia-methanol in dichloromethane. Concentrate filtrate in vacuo to obtain the title compound (16.6 g, 96%) as a thick brownish oil: mass spectrum (APCI): m/z 311.2 (M+1).

### Example 112

### (S)-(1,4-Dibenzyl-piperazin-2-yl)-acetaldehyde

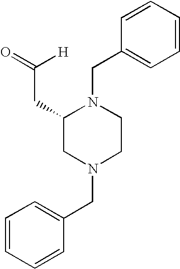

Combine a solution of oxalyl chloride (0.1.83 mL, 21.0 mmol) in dichloromethane (20 mL) with a solution of dimethyl sulfoxide (2.34 mL, 33.0 mmol) in dichloromethane (10 mL) at −78° C. and stir for 15 minutes. Add a solution of (S)-2-(1,4-dibenzylpiperazin-2-yl)ethanol (0.60 g, 1.82 mmol) in dichloromethane (10 mL) at −78° C. via cannula. Stir at −78° C. for one hour, add triethylamine (10.5 mL, 75.0 mmol) and warm to room temperature overnight. Dilute the mixture with saturated aqueous sodium bicarbonate and extract three times with dichloromethane. Combine the organic layers, dry over sodium sulfate and concentrate under reduced pressure. Purify by flash chromatography, eluting with a step gradient starting with dichioromethane going to 6% 2N ammonia-methanol in dichloromethane to obtain the title compound (3.66 g, 11.9 mmol, 79%) as a brown oil: mass spectrum (APCI): m/z=309.4 (M+1).

### Example 113

### (S)-2-Piperazin-2-yl-ethanol

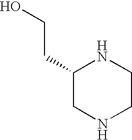

Dissolve (S)-2-(1,4-dibenzylpiperazin-2-yl)ethanol (11.7 g, 0.038 mol) in 380 mL of ethanol, add 10% palladium on carbon (3.7 g of wet reagent, 50% by weight) as a suspension in a few mL of ethanol. Add excess ammonium formate (16.8 g, 0.27 mol) all at once. Heat at reflux for 5 h, cool to room temperature and filter through a celite pad, washing well with ethanol. Concentrate filtrate in vacuo to provide the title compound (5 g, quantitative) as a cloudy residue. Use directly on the next step without further purification: mass spectrum (APCI): m/z=131.1 (M+1).

### Example 114

### (S)-2-(2-Hydroxyethyl)piperazine-1,4-dicarboxylic acid di-tert-butyl ester

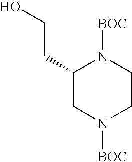

Dissolve (S)-2-piperazin-2-yl-ethanol (4 g, 30.7 mmol) in 150 mL of dichloromethane, add a few mL of ethanol to dissolve material. Add di-tert-butyl dicarbonate (28 g, 0.13 mol) in two portions, at the beginning and again after stirring at room temperature for 4 h. Pour mixture onto saturated aqueous sodium bicarbonate, extract with dichloromethane. Combine organic extracts, dry over sodium sulfate, filter, and concentrate filtrate in vacuo to a residue. Purification via silica gel chromatography eluting with a step gradient of 2% 2N ammonia-methanol in dichloromethane, 4% and 6% to obtain the title compound as a yellow oil which solidifies upon standing: mass spectrum (FAB): m/z=331.20.

### Example 115

### (S)-2-(2-Oxoethyl)piperazine-1,4-dicarboxylic acid di-tert-butyl ester

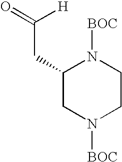

Combine a solution of oxalyl chloride (0.238 mL, 2.72 mmol) in dichloromethane (25 mL) with dimethyl sulfoxide (0.322 mL, 4.54 mmol) at −78° C. and stir. After 15 minutes, add a solution of (S)-2-(2-hydroxyethyl)piperazine-1,4-dicarboxylic acid di-tert-butyl ester (0.60 g, 1.82 mmol) in dichloromethane at −78° C. via cannula and stir at −78° C. After one hour, add triethylamine (1.27 mL, 9.08 mmol) and warm to room temperature overnight. Dilute the mixture with saturated aqueous sodium bicarbonate and extract three times with dichloromethane. Combine the organic layers, dry over sodium sulfate and concentrate under reduced pressure to give the title compound: mass spectrum (APCI): m/z=129.1 (M+1-2BOC).

### Example 116

### (S)-2-(2-Methoxyethyl)piperazine

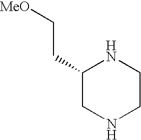

Add sodium hydride (0.675 g, 16.9 mmol) in portions to a 0° C. solution of (S)-2-(2-hydroxyethyl)piperazine-1,4-dicarboxylic acid di-tert-butyl ester (3.72 g, 11.3 mmol) in tetrahydrofuran (50 mL) and stir. After 20 minutes, add methyl iodide (1.4 mL, 22.5 mmol) dropwise. Allow the mixture to reach room temperature overnight, dilute with saturated ammonium chloride and extract three times with ethyl acetate. Combine the organic layers, dry over sodium sulfate and concentrate under reduced pressure. Purify by flash chromatography, eluting with a step gradient starting with dichloromethane up to 7% 2N ammonia-methanol in dichloromethane to obtain 2-(2-methoxyethyl)-piperazine-1,4-dicarboxylic acid di-tert-butyl ester. To the above material in dichloromethane (50 mL) add trifluoroacetic acid (15 mL) at 0° C. and stir. After 30 minutes, allow the mixture to warm up to room temperature and stir. After 1 h, remove solvent in vacuo to yield a golden yellow oil. Dilute the residue with methanol and apply to a 30 g SCX column. Wash the column with methanol, elute with 2N ammonia in methanol to obtain the title compound as a thick, colorless oil (1.13 g, 7.84 mmol, 89%): mass spectrum (APCI): m/z=145.2 (M+1).

### Example 117

### 3-(S)-Phenethyl-piperazine-2,5-dione

Add sequentially, glycine methyl ester hydrochloride (4.51 g, 35.9 mmol), 1-ethyl-(3-dimethyl-aminopropyl)carbodiimide hydrochloride (8.23 g, 42.9 mmol), 1-hydroxybenzotriazole monohydrate (5.81 g, 43.0 mmol), and triethylamine (10.0 mL, 71.7 mmol) to a solution of 2-tert-butoxycarbonylamino4-(S)-phenyl-butyric acid (10.0 g, 35.8 mmol) in methylene chloride (30 mL) at 0° C. Stir the mixture at room temperature overnight and concentrate. Partition the residue between ethyl acetate and aqueous 2N HCl (100 mL). Wash the organic layer with 10% K₂CO₃, dry (MgSO₄), and concentrate to provide the (2-tert-butoxycarbonylamino-4-(S)-phenyl-butyrylamino)-acetic acid methyl ester as a clear oil (12.3 g, 98%): ¹H NMR (CDCl₃): δ1.45 (s, 9H), 1.95 (m, 1H), 2.20 (m, 1H), 2.71 (t, 2H), 3.76 (s, 3H), 4.04 (d, 2H), 4.16 (m, 1H), 5.00 (d, 1H), 6.57 (t, 1H), 7.17-7.32 (m, 5H).

Add trifluoroacetic acid (30 mL) to (2-tert-butoxycarbonylamino-4-(S)-phenyl-butyrylamino)-acetic acid methyl ester (14.4 g). Stir one hour at room temperature and concentrate to afford (2-amino-4-(S)-phenyl-butyrylamino)-acetic acid methyl ester trifluoroacetate as an amber oil: ¹H NMR (D₂O): δ2.09 (m, 2H), 2.67 (m, 2H), 3.61 (s, 3H), 3.82 (d, 1H), 3.91 (d, 1H), 3.95 (m, 1H), 7.13-7.28 (m, 5H).

Add methanol (200 mL) and Et₃N (30 mL) to the crude trifluoroacetate salt and reflux the solution. At 2 hours, white crystals begin to form. Reflux for an additional 2 hours and cool in an ice bath and filter. Wash the crystals with cold MeOH and hexanes to afford the title compound as white crystals (6.6 g, 74%): ¹H NMR (DMSO-d6): δ1.97 (m, 2H), 2.63 (m, 2H), 3.70 (d, 1H), 3.75 (m, 1H), 3.81 (d, 1H), 7.13-7.32 (m, 5H), 8.03 (bs, 1H), 8.32 (bs, 1H).

### Example 118

### 2-(S)-Phenethyl-piperazine

Add 3-(S)-phenethyl-piperazine-2,5-dione (2.5 g, 11 mmol) portionwise to lithium aluminum hydride (1.75 g, 46 mmol) in THF (46 mL). Reflux the resulting suspension for an hour and cool to 0° C. Add sodium sulfate decahydrate carefully until hydrogen evolution ceases and stir the mixture for an additional three hours at room temperature and filter. Wash the solids with THF several times. Combine the filtrates, concentrate, and recrystallize the residue with THF/pentane to afford 2-(S)-phenethyl-piperazine as white crystals (1.9 g, 90%): mp 114-115° C.; ¹H NMR (CDCl₃): δ 1.58-1,70 (m, 2H), 2.41 (dd, 1H), 2.58-3.02 (m, 8H), 7.16-7.30 (m, 5H); MS (APCI) m/z (rel intensity) 191 (100).

### Example 118a

### 2-(S)-Phenmethyl-piperazine

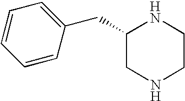

By following a similar method to Example 117 and 118 the title compound was prepared: mp 65-67° C.; ¹H NMR (CDCl₃): δ 2.47-2.57 (m, 2H), 2.67-3.00 (m, 7H), 7.16-7.30 (m, 5H); MS (APCI) m/z (rel intensity) 177 (100).

### Example 119

### (S)-1,4-Dibenzyl-2-phenethyl-piperazine

Combine (S)-1,4-dibenzyl-2-vinyl-piperazine (4.9 g, 16.63 mmol) and 9-borabicyclo[3.3.1]nonane (199.6 ml, 99.78 mmol, 0.5 M in THF) and stir at ambient temperature. After 24 hours, add iodo-benzene (5.1 g, 24.95 mmol), triphenylphosphine (697.9 mg, 2.66 mmol), tetrakis(triphenylphosphine) palladium(0)(384.3 mg, 0.33 mmol), and 3N NaOH (13.7 mL) and stir at 60° C. After 22 hours, dilute the mixture with ethyl acetate and wash it with 1N sulfuric acid. Adjust the pH to 14, extract with ethyl acetate, and combine, wash (brine), dry (sodium sulfate), and reduce the extracts to residue. Purify the residue on silica gel using a ethyl acetate/hexanes gradient.(5:95 to 10:90) to give (5.15 g, 84%) of the title compound as a white solid: mp 86-90° C.; mass spectrum (ion spray): m/z=371.3 (M+1).

### Example 120

### (S)-2-Phenethyl-piperazine

Combine (S)-1,4-dibenzyl-2-phenethyl-piperazine (5.15 g, 13.90 mmol), ammonium formate (4.38 g, 69.49 mmol), 5% Pd/C (672.5 mg), and ethanol (100 mL). Stir and heat the mixture at reflux. After 3 hours, cool to ambient temperature and remove the catalyst by vacuum filtration through celite. Reduce the filtrate to residue and purify by silica gel chromatography using dichloromethane/7N ammonia in methanol (90:10) to give the title compound as a white solid: mp 116-119° C.; mass spectrum (ion spray): m/z=191.2 (M+1); Analysis for C₁₂H₁₈N₂: calcd: C, 75.74; H, 9.53; N, 14.72; found: C, 75.90; H, 9.57; N, 14.59.

### Example 121

### (S)-1,4-Dibenzyl-2-(2-(4-methoxy-phenyl)-ethyl)-piperazine

Combine (s)-1,4-dibenzyl-2-vinyl-piperazine (2.0 g, 6.84 mmol) and 9-borabicyclo[3.3.1]nonane (82.1 mL, 41.04 mmol, 0.5 M in THF) and stir at ambient temperature. After 6 hours and 30 minutes, add 1-iodo-4-methoxy-benzene (2.4 g, 10.26 mmol), triphenylphosphine (287.0 mg, 1.09 mmol), tetrakis(triphenylphosphine) palladium(0)(158.0 mg, 0.14 mmol), and 3N NaOH (5.6 mL) and stir at 60° C. After 22 hours, add ethanolamine (10.0 ml) and dilute the mixture with water. Extract with ethyl acetate and combine, wash (brine), dry (sodium sulfate), and reduce the extracts to residue. Purify the residue on silica gel using ethyl acetate/hexanes (5:95) to give a yellow oil. Dissolve the yellow oil in acetic acid/methanol (1:9) and apply to an SCX column. Wash the column with methanol followed by 7N ammonia in methanol to give the title compound: mp 88-91° C.; mass spectrum (ion spray): m/z=401.4 (M+1).

### Example 122

### (S)-2-(2-(4-Methoxy-phenyl)-ethyl)-piperazine

Combine (S)-1,4-dibenzyl-2-(2-(4-methoxy-phenyl)-ethyl)-piperazine (1.34 g, 3.33 mmol), ammonium formate (1.05 g, 16.67 mmol), 5% Pd/C (161.2 mg),. and ethanol (100 mL). Stir and heat the mixture at reflux. After 3 hours, cool to ambient temperature and remove the catalyst by vacuum filtration through celite. Reduce the filtrate to residue and purify it on silica gel using dichloromethane/2N ammonia in methanol (80:20) to give (689.3 mg, 94%) of the title compound as a white solid: mp 125-130° C.; mass spectrum (ion spray): m/z=221.1 (M+1); Analysis for C₁₃H₂₀N₂O: calcd: C, 70.87; H, 9.15; N, 12.72; found: C, 70.58; H, 9.05; N, 12.61.

### Example 123

### (S)-1,4-Dibenzyl-2-[2-(4-tifluoromethyl-phenyl)-ethyl]-piperazine

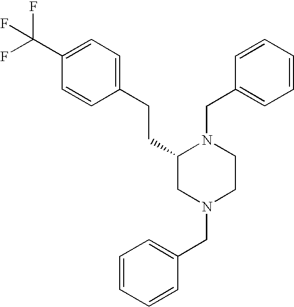

Combine (S)-1,4-dibenzyl-2-vinyl-piperazine (6.0 g, 20.52 mmol) and 9-borabicyclo[3.3.1]nonane (164.1 ml, 82.07 mmol, 0.5 M in THF) and stir at ambient temperature. After 24 hrs, add 1-iodo-4-trifluoromethyl-benzene (8.37 g, 30.78 mmol), triphenylphosphine (861.0 mg, 3.28 mmol), tetrakis(triphenylphosphine). palladium(0)(474.1 mg, 0.41 mmol), and 3N NaOH (16.8 ml) and stir at 60°0. After 22 hrs, remove the THF under vacuum, stir the residue in 2N NaOH, and extract with diethyl ether. Wash the organic with 1N H₂SO₄ then adjust the aqueous to pH 14. Extract the aqueous with diethyl ether and combine, wash (brine), dry (sodium sulfate), and reduce the extracts to residue. Recrystallize the residue in warm ethanol to give 3.34 g (37%) of the title compound: mass spectrum (ion spray): mp 71°-75°; ml/z=439.2 (M+1); Analysis for C₂₇H₂₉F₃N₂: calcd: C, 73.95; H, 6.67; N, 6.39; found: C, 74.15; H, 6.72; N, 6.52.

### Example 124

### (S)-2-[2-(4-Trifluoromethyl-phenyl)-ethyl]-piperazine

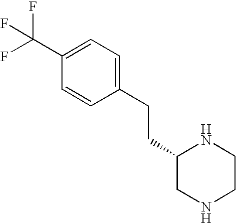

Combine (S)-1,4-dibenzyl-2-[2-(4-trifluoromethyl-phenyl)-ethyl]-piperazine (3.34 g, 7.62 mmol), ammonium formate (2.40 g, 38.09 mmol), 5% Pd/C (368.7 mg), and ethanol (100 ml). Stir and heat the mixture at reflux. After 3 hrs, cool to ambient temperature and remove the catalyst by vacuum filtration through celite. Reduce the filtrate to residue and then dissolve it in dichloromethane. Wash the organic with 1N NaOH and then combine, wash (brine), dry (sodium sulfate), and reduce the extracts to residue to give 1.83 g (93%) of the title compound as an off-white solid: mp 135°-141°; mass spectrum (ion spray): m/z=259.2 (M+1).

### Example 125

### 10-(S)-(3-Benzyl-piperazin-1-yl)-2-methyl-4H-3-thia-9-aza-benzo[f]azulene succinate salt

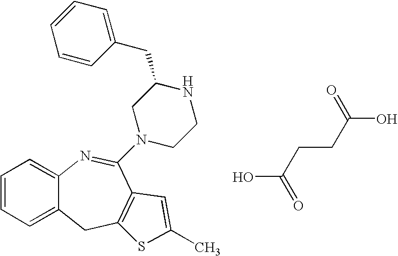

Add 2-methyl-4,9-dihydro-3-thia-9-aza-benzo[f]azulen-10-one (1.34 g, 5.85 mmol, prepared according to Eur. J. Med. Chem. P. 391-398, 1981) to phosphorus oxychloride (40 mL) and reflux for 3 hours. Allow the mixture to cool to ambient temperature then concentrate under reduced pressure. Treat the reaction mixture with saturated sodium bicarbonate and extract with dichloromethane. Combine the organic layers, dry over sodium sulfate and concentrate under reduced pressure. Dissolve the crude residue and 2-(S)-benzyl-piperazine (2.06 g, 11.7 mmol) in dioxane (40 mL) and pyridine (12 mL). Reflux the mixture for 42 hours then cool to ambient temperature. Treat the reaction mixture with ammonium hydroxide and extract with dichloromethane. Combine the organic layers, dry over sodium sulfate and concentrate under reduced pressure. Purification by flash chromatography, eluting with dichloromethane/dichloromethane:methanol:ammonia (94%:6%:0.35 M .gradient)gives 10-(S)-(3-benzyl-piperazin-1-yl)-2-methyl-4H-3-thia-9-aza-benzo[f]azulene. Treating the resulting semisolid in methanol with succinic acid (0.46 g, 3.90 mmol) in methanol and concentrating under reduced pressure gives the title compound: mass spectrum (m/e): 388(M⁺+1 of free base); Analysis for C₂₄H₂₅N₃S.C₄H₆O₄.0.5H₂O: calcd: C, 65.35; H, 6.27; N, 8.17; found: C, 65.13; H, 6.17; N, 8.05.

### Example 127

### 10-(S)-(3-Benzyl-4-methyl-pinerazin-1-yl)-2-methyl-4H-3-thia-9-aza-benzo[f]azulene succinate salt

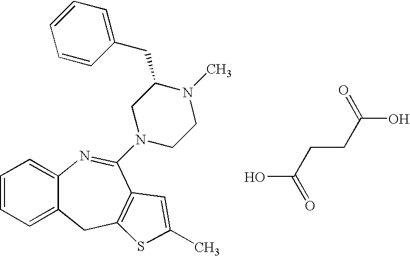

Add sodium triacetoxyborohydride. (0.313 g, 1.48 mmol) and formaldehyde (87 μL, 1.08 mmol, 37%) to a solution of 10-(S)-(3-benzyl-piperazin-1-yl)-2-methyl-4H-3-thia-9-aza-benzo[f]azulene (0.382 g, 0.986 mmol) in dichloroethane (40 mL) and stir the mixture for two hours. Treat the reaction mixture with saturated sodium bicarbonate and extract with dichloromethane. Combine the organic layers, dry over sodium sulfate and concentrate under reduced pressure. Purification by flash chromatography, eluting with dichloromethane/dichloromethane:methanol:ammonia (94%:6%:0.35 M gradient)gives 10-(S)-(3-benzyl-4-methyl-piperazin-1-yl)-2-methyl-4H-3-thia-9-aza-benzo[f]azulene (0.354 g, 0.882 mmol, 89%). Treating the resulting oil in methanol with succinic acid (0.104 g, 0.882 mmol) in methanol and concentrating under reduced pressure gives the title compound. mass spectrum (m/e): 402(M⁺+1 of free base); Analysis for C₂₅H₂₇N₃S.C₄H₆O₄: calcd: C, 67.03; H, 6.40; N, 8.09; found: C, 66.73; H, 6.50; N, 7.97.

### Example 128

### 2-Methyl-10-(S)-(3-phenethyl-piperazin-1-yl)-4H-3-thia-9-aza-benzo[f]azulene succinate salt

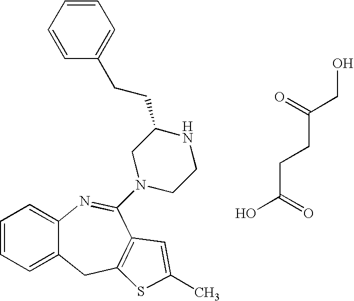

Add 2-methyl-4,9-dihydro-3-thia-9-aza-benzo[f]azulen-10-one (0.352 g, 1.54 mmol) to phosphorus oxychloride (10 mL)and reflux for 3 hours. Allow the mixture to cool to ambient temperature then concentrate under reduced pressure. Treat the reaction mixture with toluene then concentrate under reduced pressure. Treat the reaction mixture with toluene again then concentrate under reduced pressure. Dissolve the crude residue and 2-(S)-phenethyl-piperazine (0.585 g, 3.07 mmol) in dioxane (10 mL). Reflux the mixture for 20 hours then cool to ambient temperature. Treat the reaction mixture with ammonium hydroxide and extract with dichloromethane. Combine the organic layers, dry over sodium sulfate and concentrate under reduced pressure. Purification by flash chromatography, eluting with dichloromethane/dichloromethane:methanol:ammonia (95%:5%:0.35 M gradient)gives 2-methyl-10-(S)-(3-phenethyl-piperazin-1-yl)-4H-3-thia-9-aza-benzo[f]azulene. Treating the resulting solid in methanol with succinic acid (91 mg, 0.772 mmol) in methanol and concentrating under reduced pressure gives the title compound: mass spectrum (m/e): 402(M⁺+1 of free base); Analysis for C₂₅H,₂₇N₃S.C₄H₆O₄.H₂O: calcd: C, 64.78; H, 6.56; N, 7.82; found: C, 64.56; H, 6.37; N, 7.47.

### Example 129

### 2-Methyl-10-(S)-(4-methyl-3-phenethyl-piperazin-1-yl)-4H-3-thia-9-aza-benzo[f]azulene succinate salt

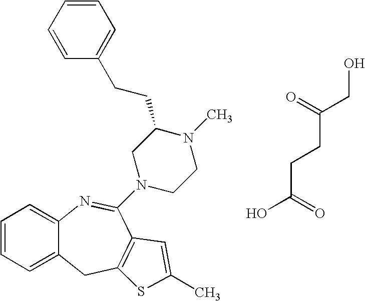

Add sodium triacetoxyborohydride (0.218 g, 1.03 mmol) and formaldehyde (61 μL, 0.756 mmol, 37%) to a solution of 2-methyl-10-(S)-(3-phenethyl-piperazin-1-yl)-4H-3-thia-9-aza-benzo[f]azulene (0.276 g, 0.687 mmol) in dichloroethane (20 mL) and stir the mixture for two hours. Treat the reaction mixture with saturated sodium bicarbonate and extract with dichloromethane. Combine the organic layers, dry over sodium sulfate and concentrate under reduced pressure. Purification by flash chromatography, eluting with dichloromethane/dichloromethane:methanol:ammonia (96%:4%:0.35 M gradient) gives 2-methyl-10-(S)-(4-methyl-3-phenethyl-piperazin-1-yl)-4H-3-thia-9-aza-benzo[f]azulene. Treating the resulting oil in methanol with succinic acid (0.073 g, 0.621 mmol) in methanol and concentrating under reduced pressure gives the title compound: mass spectrum (m/e): 416(M⁺+1 of free base); Analysis for C₂₆H₂₉N₃S.C₄H₆O₄.0.6H₂O: calcd: C, 66.18; H, 6.70; N, 7.72; found: C, 65.80; H, 6.32; N, 7.60.

### Example 130

### (S)-2-[4-(2-Methyl-4H-3-thia-9-azabenzo[f]azulen-10-yl) piperazin-2-yl]ethanol dihydrochloride

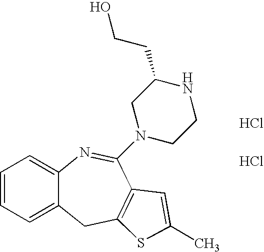

Combine 2-methyl-4,9-dihydro-3-thia-9-aza-benzo[f]azulen-10-one (0.275 g, 1.20 mmol) and phosphorus oxychloride (6 mL) and heat at reflux for three hours. Cool the mixture and evaporate, then dilute the residue with saturated aqueous sodium bicarbonate and extract four times with dichlorornethane. Combine the organic layers, dry over sodium sulfate and concentrate under reduced pressure. Combine the residue, pyridine (3 mL) and dioxane (3 mL) along with (S)-2-piperazin-2-yl-ethanol (1.372 g, 10.5 mmol) and heat at reflux for 18 hours. Evaporate the solution and apply the material to 40 g of SCX resin, then elute with a step gradient starting with methanol going to 5% 2N ammonia-methanol in dichloromethane. Purification twice by flash chromatography, eluting with a step gradient starting with dichloromethane going to 10% 2N ammonia-methanol in dichloromethane, gives the free base of the title compound (0.018 g, 0.053 mmol, 4%). Isolate as the dihydrochloride salt: Mass spectrum (APCI): m/z=342.1 (M+1 of free base).

### Example 131

### (S)-2-[2-[4-(2-Methyl-4H-3-thia-9-aza-benzo[f]azulen-10-yl)-2-phenethyl-piperazin-1-yl]-ethoxy]-ethanol hydrochloride

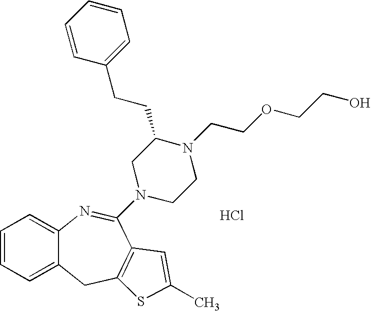

Combine (S)-2-methyl-10-(3-phenethyl-piperazin-1-yl)-4H-3-thia-9-aza-benzo[f]azulene (0.08 g, 0.2 mmol) in toluene at room temperature, and add 2-(2-chloroethoxyl)-ethanol (0.05 g, 0.4 mmol) followed by K₂CO₃ (0.28 g, 2.0 mmol) and KI (0.33 g, 2.0 mmol) and reflux the resulting mixture. After 4 hours, cool the reaction mixture to room temperature, dilute with ethyl acetate (50 ml), wash with brine, dry (MgSO₄) and evaporate under reduced pressure. Purification by flash chromatography, eluting with dichloromethane/dichloromethane:methanol:ammonia (94%:6%:0.35 M gradient) gives (S)-2-[2-[4(2-methyl-4H-3-thia-9-aza-benzo[f]azulen-10-yl)-2-phenethyl-piperazin-1-yl]-ethoxy]-ethanol. Treating the resulting oil in ethyl acetate with 1 N HCl in ether (0.5 ml) and concentrating under reduced pressure gives the title compound: mass spectrum: 490.2.

### Example 132

### (S)-1-[2-[4-(2-Methyl-4H-3-thia-9-aza-benzo[f]azulen-10-yl)-2-phenethyl-piperazin-1-yl]-ethyl]-imidazolidin-2-one hydrochloride

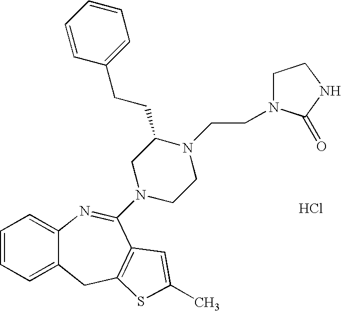

Combine (S)-2-methyl-10-(3-phenethyl-piperazin-1-yl)-4H-3-thia-9-aza-benzo[f]azulene (0.08 g, 0.2 mmol) and toluene and stir at room temperature, and add 4-(2-chloro-ethyl)-imidazolidin-2-one (0.06 g, 0.4 mmol) followed by K₂CO₃ (0.28 g, 2.0 mmol) and KI (0.33 g, 2.0 mmol) and reflux the resulting mixture. After for 4 hours, cool the reaction mixture to room temperature, dilute with ethyl acetate (50 ml), wash with brine, dry (MgSO₄) and evaporate under reduced pressure. Purification by flash chromatography, eluting with dichloromethane/dichloromethane:methanol:ammonia (94%:6%:0.35 M gradient) gives (S)-1-[2-[4-(2-methyl-4H-3-thia-9-aza-benzo[f]azulen-10-yl)-2-phenethyl-piperazin-1-yl]-ethyl]-imidazolidin-2-one. Treating the resulting oil in ethyl acetate with 1 N HCl in ether (0.5 ml) and concentrating under reduced pressure gives the title compound: mass spectrum: 514.6.

### Example 133

### (S)-5-[4-(2-Methyl-4H-3-thia-9-aza-benzo[f]azulen-10-yl)-2-phenethyl-piperazin-1-yl]-pentanenitrile

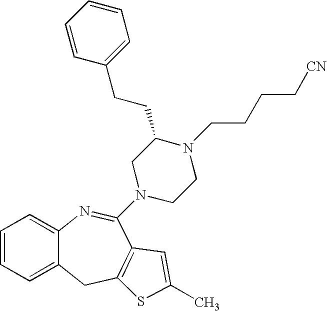

Combine 2-methyl-10-(3-phenethyl-piperazin-1-yl)-4H-3-thia-9-aza-benzo[f]azulene (0.08 g, 0.2 mmol) and toluene at room temperature, add 2-(2-chloroethoxyl)-ethanol (0.05 g, 0.4 mmol) followed by K₂CO₃ (0.28 g, 2.0 mmol) and KI (0.33 g, 2.0 mmol) and reflux the resulting mixture. After 4 hours, cool the reaction mixture to room temperature, dilute with ethyl acetate (50 ml), wash with brine, dry (MgSO₄) and evaporate under reduced pressure. Purification by flash chromatography, eluting with dichloromethane/dichloromethane:methanol:ammonia (94%:6%:0.35 M gradient)gives 2-[2-[4-(2-Methyl-4H-3-thia-9-aza-benzo[f]azulen-10-yl)-2-phenethyl-piperazin-1-yl]-ethoxy]-ethanol (0.08 g, 85%). Treating the resulting oil in ethyl acetate with 1 N HCl in ether (0.5 ml) and concentrating under reduced pressure gives the title compound: mass spectrum: 490.2.

### Example 134

### (S)-2-Methyl-10-[4-[4-(1-methyl-1H-tetrazol-5-yl)-butyl]-3-phenethyl-piperazin-1-yl]-4H-3-thia-9-aza-benzo[f]azulene hydrochloride

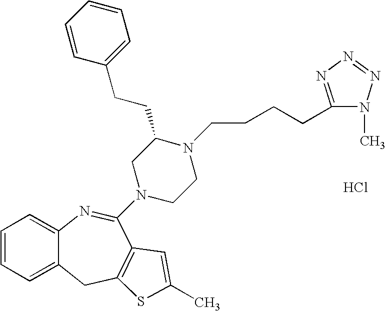

Dissolve (S)-5-[4-(2-methyl-4H-3-thia-9-aza-benzo[f]azulen-10-yl)-2-phenethyl-piperazin-1-yl]-pentanenitrile (0.13 g, 0.27 mmol) in toluene (5.0 ml) at room temperature and add TMS-N₃ (0.31 g, 2.7 mmol)and MeSnO (0.022 g, 0.135 mmole) and refluxing the resulting solution. After 18 hours, cool the reaction mixture to room temperature, evaporate under reduced pressure. Purification by flash chromatography, eluting with dichloromethane/dichloromethane:methanol:ammonia (94%:6%:0.35 M gradient) gives (S)-2-methyl-10-[4-[2-(1-methyl-1H-tetrazol-5-yl)-ethyl]-3-phenethyl-piperazin-1-yl]-4H-3-thia-9-aza-benzo[f]azulene. Treating the resulting oil in ethyl acetate with 1 N HCl in ether (0.5 ml) and concentrating under reduced pressure gives the title compound: mass spectrum: 540.2.

### Example 135

### (S)-2-Methyl-10-[3-[2-(4-trifluoromethyl-phenyl)-ethyl]-piperazin-1-yl]-4H-3-thia-9-aza-benzo[f]azulene succinate salt

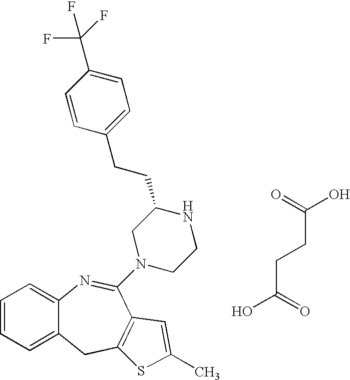

By using a method similar to Example 125, using 2-methyl-4,9-dihydro-3-thia-9-aza-benzo[f]azulen-10-one and (S)-2-(4-trifluoro-phenyl)-ethyl]-piperazine gives the title compound: mass spectrum (m/e): 470.2.

### Example 136

### (S)-2-Methyl-10-[4-methyl-3-[2-(4-trifluoromethyl-phenyl)-ethyl]-piperazin-1-yl]-4H-3-thia-9-aza-benzo[f]azulene

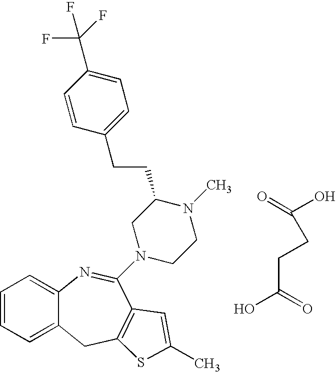

By using a method similar to Example 127, using (S)-2-Methyl-10-[3-[2-(4-trifluoromethyl-phenyl)-ethyl]-piperazin-1-yl]-4H-3-thia-9-aza-benzo[f]azulene succinate salt gives the title compound was prepared: mass spectrum: 484.1.

### Example 146

### (S)-2-Isopropyl-10-(3-phenethyl-piperazin-1-yl)-4H-3-thia-9-aza-benzo[f]azulene succinate

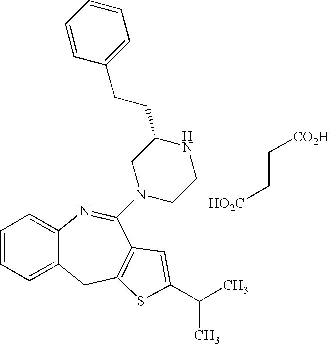

By using a method similar to Example 166, using 2-isopropyl-4,9-dihydro-3-thia-9-aza-benzo[f]azulen-10-one (0.750 g, 2.90 mmol) and (S)-2-phenethyl-piperazine (0.609 g, 3.20 mmol) to obtain the free base which is converted to the succinate salt as previously described to give the title compound: Mass Spectrum (m/e): 430.5 (M+1); free base.

### Example 147

### (S)-2-Isopropyl-10-(4-methyl-3-phenethyl-piperazin-1-yl)-4H-3-thia-9-aza-benzo[f]azulene succinate

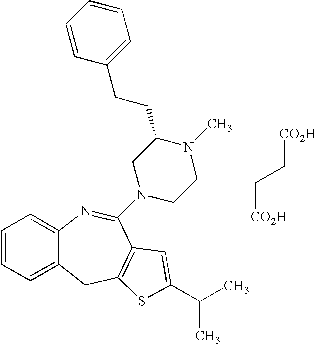

By using a method similar to Example 167, using (S)-2-isopropyl-10-(3-phenethyl-piperazin-1-yl)-4H-3-thia-9-aza-benzo[f]azulene (0.304 g, 0.71 mmole) to obtain the free base of the title compound which is converted to the succinate salt as previously described to give the title compound: Mass Spectum (m/e): 444.6 (M+1); free base.

### Example 148

### (S)-10-(3-Benzyl-piperazin-1-yl)-2-isopropyl-4H-3-thia-9-aza-benzo[f]azulene succinate

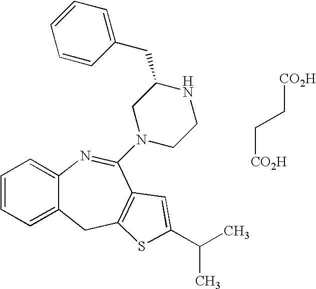

By using a method similar to Example 168, using 2-isopropyl-4,9-dihydro-3-thia-9-aza-benzo[f]azulen-10-one(0.993 g, 3.86 mmol) and (S)-2-benzyl-piperazine (1.33 g, 7.54 mmol obtained from Rhodia ChiRex) to obtain the free base which is converted to the succinate salt as previously described to give the title compound: Mass Spectrum (m/e): 416.6 (M+1); free base.

### Example 149

### (S)-10-(3-Benzyl-4-methyl-piperazin-1-yl)-2-isopropyl-4H-3-thia-9-aza-benzo[f]azulene succinate

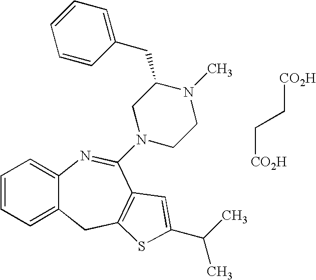

By using a method similar to Example 169, using (S)-10-(3-benzyl-piperazin-1-yl)-2-isopropyl-4H-3-thia-9-aza-benzo[f]azulene (0.139 g, 0.334 mmol) to obtain the free base of the title compound and which is converted to the succinate salt as previously described to give the title compound: Mass Spectrum (m/e): 430.6 (M+1); free base.

### Example 150

### (S)-10-(3-Benzyl-4-2-(2-hydroxy-ethoxy)-ethyl]-piperazin-1-yl)-2-isopropyl-4H-3-thia-9-aza-benzo[f]azulene succinate

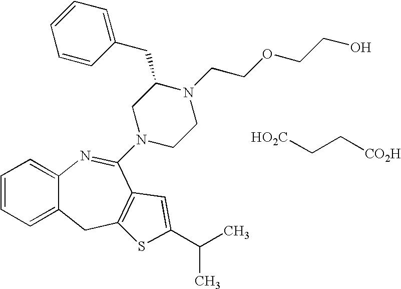

By using a method similar to Example 170, using (S)-10-(3-benzyl-piperazin-1-yl)-2-isopropyl-4H-3-thia-9-aza-benzo[f]azulene (0.100 g, 0.241 mmol) to obtain the free base of the title compound which is converted to the succinate salt as previously described to give the title compound: Mass Spectrum (m/e): 504.7 (M+1); free base.

### Example 151

### (S)-10-(3-Phenethyl-4-[2-(2-hydroxy-ethoxy)-ethyl]-piperazin-1-yl)-2-isopropyl-4H-3-thia-9-aza-benzo[f]azulene succinate

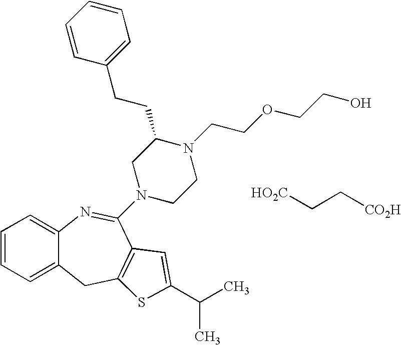

By using a method similar to Example 170, using (S)-2-isopropyl-10-(3-phenethyl-piperazin-1-yl)-4H-3-thia-9-aza-benzo[f]azulene (0.150 g, 0.349 mmol) to obtain the free base of the title compound which is converted to the succinate salt: Mass Spectrum (m/e): 518.7 (M+1); free base.

### Example 152

### (S)-10-(3,4-Diphenethyl-piperazin-1-yl)-2-isopropyl-4H-3-thia-9-aza-benzo[f]azulene succinate

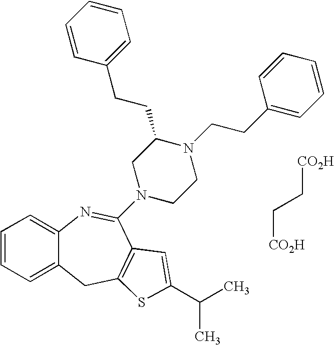

By using a method similar to Example 147, using (S)-2-isopropyl-10-(3-phenethyl-piperazin-1-yl)-4H-3-thia-9-aza-benzo[f]azulene (0.122 g, 0.284 mmol) and phenylacetaldehyde (0.068 g, 0.568 mmol) to obtain the title compound, which is converted to the succinate salt as previously described: Mass Spectrum (m/e): 534.7 (M+1); free base.

### Example 153

### (S)-10-(3-Phenethyl-4-[2-hydroxyethyl]-piperazin-1-yl)-2-isopropyl-4H-3-thia-9-aza-benzo[f]azulene succinate

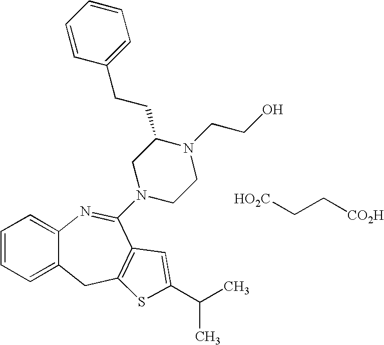

By using a method similar to Example 151, using (S)-2-isopropyl-10-(3-phenethyl-piperazin-1-yl)-4H-3-thia-9-aza-benzo[f]azulene (0.150 g, 0.349 mmol) and 2-bromoethanol (0.087 g, 0.698 mmole) to obtain the free base of the title compound which is converted to the succinate salt as previously described: Mass Spectrum (m/e): 474.7 (M+1); free base.

### Example 154

### (S)-10-[3-[2-(4-Methoxy-phenyl)-ethyl]-4-methyl-piperazin-1-yl]-2-methyl-4H-3-thia-9-aza-benzo[f]azulene dihydrochloride

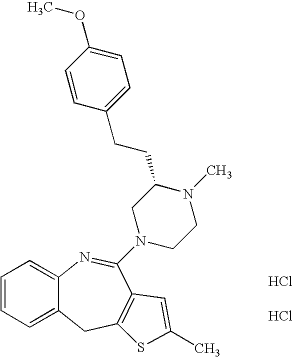

By using a method of Example 190, using (S)-10-[3-[2-(4-methoxy-phenyl)-ethyl]-piperazin-1-yl]-2-methyl-4H-3-thia-9-aza-benzo[f]azulene to give the title compound: mass spectrum: 445.1.

### Example 155

### 5-(4-Methyl-(2-R³⁻Y-Alk)-piperazin-1-yl)-11H-12-thia-6-aza-dibenzo[a,f]azulene hydrochloride

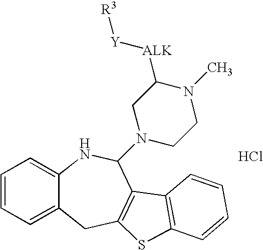

Treat appropriate amide (0.88 mmol) with P₂O5 (0.5 g) in POCl₃ (10 ml) at room temperature, refluxed the resulting solution. After 3 hours, cool to room temperature and evaporate under reduced pressure. Treat the residue with ice/H₂O (20 ml), followed by saturated NaHCO₃ (50 ml), and extract with ethyl acetate (2×50 ml). Wash the organic layer with brine, dry (MgSO₄) and evaporate under reduced pressure. Purification by flash chromatography, eluting with dichloromethane/dichloromethane; methanol:ammonia (94%:6%:0.35 M gradient) gives the desired product. Treating the resulting oil in ethyl acetate with 1 N HCl in ether (0.5 ml) and concentrating under reduced pressure gives the title compound.

By using a method similar to Example 155, the following compounds were prepared:

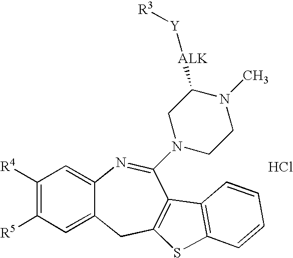

| NO.: | Alk | Y | R³ | R⁴ | R⁵ | DATA |
|---|---|---|---|---|---|---|
| 156 | CH₂CH₂ | O | CH₃ | H | H | Exact Mass spectrum (m/e): |
| | | | | | | 406.1964 |
| 159 | CH₂CH₂ | O | CH₃ | H | Cl | Exact Mass spectrum (m/e): |
| | | | | | | 440.1562 |
| 160 | CH₂CH₂ | O | CH₃ | H | F | Exact Mass spectrum (m/e): |
| | | | | | | 423.5613 |
| 161 | CH₂CH₂ | O | CH₃ | F | F | Exact Mass spectrum (m/e): |
| | | | | | | 460.1863 |

### Example 166

### (S)-2-Methyl-10-(3-phenethyl-piperazin-1-yl)-3-thia-9-aza-benzo[f]azulen-4-one succinate

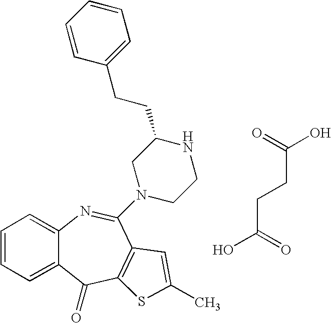

Dissolve 2-methyl-9H-3-thia-9-aza-benzo[f]azulene-4,10-dione (0.272 g, 1.12 mmol) in phosphorus oxychloride (8 mL) and heat the mixture to reflux (106° C.) for three hours. Remove the phosphorus oxychloride under reduced pressure to give a residue. Take up the residue in saturated aqueous sodium bicarbonate and extract three times with dichloromethane. Combine the organic layers, dry over sodium sulfate, and remove the solvent under reduced pressure. Take up the resulting residue in a mixture of 8 ml 1,4-dioxane, and 2 ml of pyridine. Add (S)-2-phenethyl-piperazine (0.425 g, 2.24 mmol) and heat the resulting mixture to 100° C. After 18 hours remove the solvent under reduced pressure. Dissolve the residue in dichloromethane and ammonium hydroxide (28% aqueous). Separate the organic and extract the aqueous three times with dichloromethane. Combine the organic layers and dry over sodium sulfate. Remove the solvent under reduced pressure. Purification of the residue by flash chromatography eluting with a gradient starting with 100% dichloromethane and going to 97% dichloromethane.3% 2 M ammonia in methanol gives (S)-2-Methyl-10-(3-phenethyl-piperazin-1-yl)-3-thia-9-aza-benzo[f]azulen-4one. Convert the free base to the succinate salt by dissolving the product in methanol and adding one equivalent of succinic acid, swirl or sonicate the mixture until no solid succinic acid remains, then removing the solvent under reduced pressure gives the title compound: Exact Mass: Calc. 416.1797; Found 416.1770.

### Example 167

### (S)-2-Methyl-10-(4-methyl-3-phenethyl-piperazin-1-yl)-3-thia-9-aza-benzo[f]azulen-4-one succinate

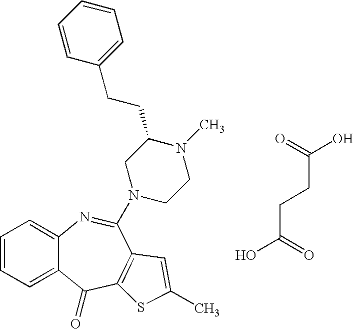

Dissolve (S)-2-methyl-10-(3-phenethyl-piperazin-1-yl)-3-thia-9-aza-benzo[f]azulen-4-one succinate (0.264 g, 0.63 mmol, free base) in dichloromethane (10 ml) and add sodium triacetoxyborohydride (0.269 g, 1.27 mmol) and formaldehyde (0.038 g, 1.27 mmol, 0.103 g of a 37% aqueous solution) stir the mixture for one hour at ambient temperature. Dilute the mixture with brine and extract three times with dichloromethane. Combine the organic layers, dry over sodium sulfate and remove the solvent under reduced pressure. Purification of the residue by flash chromatography eluting with 97% dichloromethane:3% 2 M ammonia in methanol gives the free base of the title compound, which is then converted to the succinate salt: Exact Mass: Calc. 430.1953; Found 430.1973.

### Example 168

### (S)-10-(3-Benzyl-piperazin-1-yl)-2-methyl-3-thia-9-aza-benzo[f]azulen-4-one succinate

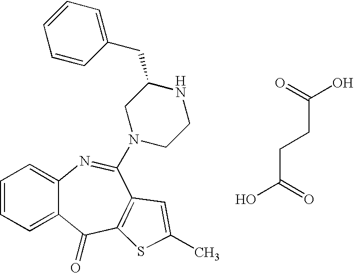

By using a method similar to Example 166, using 2-methyl-9H-3-thia-9-aza-benzo[f]azulene-4,10-dione (0.115 g, 0.47 mmol) and (S)-2-benzyl-pipepazine (obtained from Rhodia ChiRex) (0.167 g, 0.945 mmol) to obtain the free base of the title compound which is converted to succinate. salt as previously described:. Exact Mass: Calc. 401.1562; Found 401.1570.

### Example 169

### (S)-10-(3-Benzyl-4-methyl-piperazin-1-yl)-2-methyl-3-thia-9-aza-benzo[f]azulen-4-one succinate

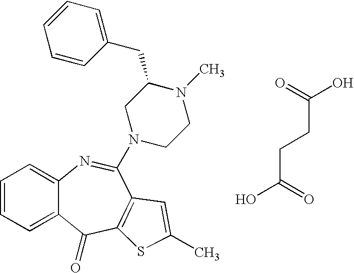

By using a method similar to Example 167, using (S)-10-(3-benzyl-piperazin-1-yl)-2-methyl-3-thia-9-aza-benzo[f]azulen-4-one succinate (0.090 g, 0.22 mmol), sodium triacetoxyborohydride (0.095 g, 0.44 mmol) and of fornaldehyde(0.013 g, 0.44 mmol, 0.036 g of the 37% aqueous solution) to obtain the free base of the title compound which is converted to succinate salt, as previously described: Exact Mass: Calc. 416.1797; Found 416.1800.

### Example 170

### (S)-10-[3-Benzyl-4-[2-(2-hydroxy-ethoxy)-ethyl]-piperazin-1-yl]-2-methyl-3-thia-9-aza-benzo[f]azulen-4-one succinate

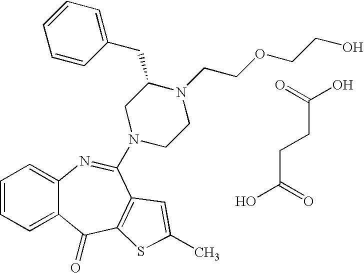

Dissolve (S)-10-(3-benzyl-piperazin-1-yl)-2-methyl-3-thia-9-aza-benzo[f]azulen-4-one succinate (0.100 g, 0.25 mmol) in 7 ml of acetonitrile, and add potassium iodide (0.207 g, 1.25 mmol), potassium carbonate (0.172 g, 1.25 mmol) and 2(2-chloroethoxy)ethanol (0.155 g, 1.25 mmol). Heat the mixture to reflux for 48 hours. After cooling to ambient temperature, pour the reaction mixture into brine and extract three times with ethyl acetate. Combine the organic layers, dry over sodium sulfate, and remove the solvent under reduced pressure. Purification of the residue via flash chromatography, eluting with a step gradient starting with dichloromethane and going to 97% dichloromethane and 3% 2 M ammonia in methanol, gives the free base of the title compound which is converted to the succinate to give the title compound: Exact Mass: Calc. 490.2164; Found 490.2157.

### Example 172

### (S)-10-[3-(2-Methoxy-ethyl)-piperazin-1-yl]-2-methyl-4H-3-thia-1,9-diaza-benzo[f]azulene succinate

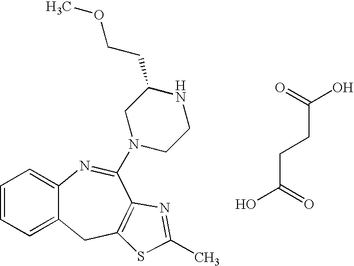

Dissolve 2-methyl-4,9-dihydro-3-thia-1,9-diaza-benzo[f]azulene-10-thione (0.105 g, 0.43 mmol) in dichloromethane (2 ml) and add in one portion methyl trifluormethanesulfonate (0.105 g, 0.64 mmol) and stir the mixture at ambient temperature. After two hours, remove the solvent under reduced pressure. Dissolve the residue dissolve in pyridine (2 ml) and add (S)-2-(2-methoxy-ethyl)-piperazine (0.092 g, 0.64 mmol). Heat the resulting mixture to reflux (115° C.) for 8 hours. Evaporate the solvent and purify the residue via flash chromatography, eluting with a step gradient starting with dichloromethane and going to 97% dichloromethane with 3% 2 M ammonia in methanol, to give the free base of the title compound (0.138 g, 0.39 mmol, 90% yield) as an off-white foam. The free base is then converted to the succinate salt to give the title compound: Exact Mass: Calc. 357.1749; Found 357.1736.

### Example 173

### (S)-10-[3-(2-Methoxy-ethyl)-4-methyl-piperazin-1-yl]-2-methyl-4H-3-thia-1,9-diaza-benzo[f]azulene succinate

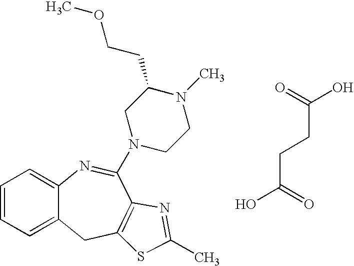

By using a method similar to Example 167, using the free base of (S)-10-[3-(2-methoxy-ethyl)-piperazin-1-yl]-2-methyl-4H-3-thia-1,9-diaza-benzo[f]azulene (0.096 g, 0.27 mmol) and purification via flash chromatography, eluting with a step gradient starting with dichloromethane and going to 97% dichloromethane with 3% 2 M ammonia in methanol, gives the free base of the title compound (0.063 g, 0.17 mmol, 63% yield) as an off-white foam. The free base is then converted to the succinate salt to give the title compound: Exact Mass: Calc. 371.1906; Found 371.1909.

### Example 174

### (S)-10-[3-[2-(4-Methoxy-phenyl)-ethyl]-piperazin-1-yl]-2-methyl-4H-3-thia-1,9-diaza-benzo[f]azulene succinate

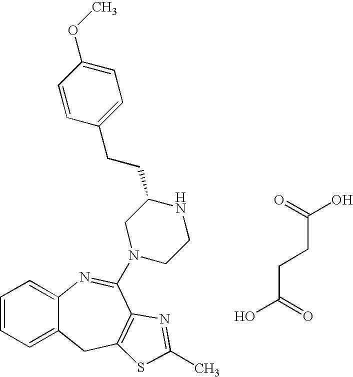

Dissolve 2-methyl-4,9-dihydro-3-thia-1,9-diaza-benzo[f]azulene-10-thione (0.164 g, 0.67 mmol), in dichloromethane (3 ml) and add in one portion methyl trifluormethanesulfonate (0.164 g, 1.00 mmol) and stir the mixture at ambient temperature. After two hours, remove the solvent under reduced pressure. Dissolve the residue dissolve in pyridine (2 ml) and add (S)-2-[2-(4-methoxy-phenyl)-ethyl]-piperazine (0.220 g, 1.00 mmol). Heat the resulting mixture to reflux (115° C.) for 8 hours. Evaporate the solvent Purification via flash chromatography, eluting with a step gradient starting with dichloromethane and going to 97% dichloromethane with 3% 2 M ammonia in methanol, to give the free base of the title compound. The free base is then converted to the succinate salt to give the title compound: Exact Mass: Calc. 433.2062; Found 433.2060.

### Example 175

### (S)-10-[3-[2-(4-Methoxy-phenyl)-ethyl]-4-methyl-piperazin-1-yl]-2-methyl-4H-3-thia-1,9-diaza-benzo[f]azulene succinate

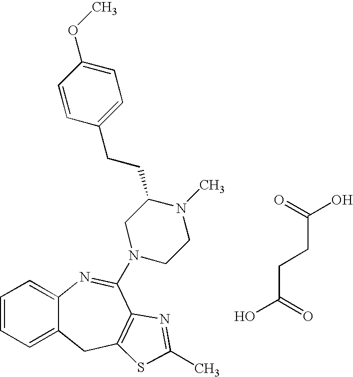

By using a method similar to Example 174, using (S)-10-[3-[2-(4-methoxy-phenyl)-ethyl]-piperazin-1-yl]-2-methyl-4H-3-thia-1,9-diaza-benzo[f]azulene (0.134 g, 0.31 mmol). Purification via flash chromatography, eluting with a step gradient starting with dichloromethane and going to 97% dichloromethane with 3% 2 M ammonia in methanol, gives the free base of the title compound. The free base is then converted to the succinate salt to give the title compound: Exact Mass: Calc. 447.2218; Found 447.2-206.

### Example 176

### (S)-10-[3-[2-(3-Fluoro-phenyl)-ethyl]-piperazin-1-yl]-2-methyl-4H-3-thia-1,9-diaza-benzo[f]azulene succinate

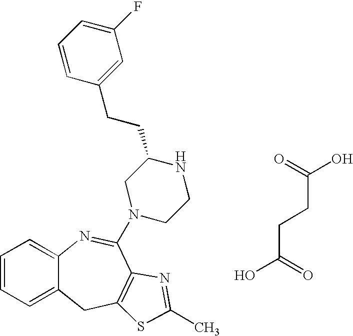

By using a method similar to Example 174, using 2-methyl-4,9-dihydro-3-thia-1,9-diaza-benzo[f]azulene-10-thione (0.070 g, 0.28 mmol) with the following changes: methyl trifluormethanesulfonate (0.065 g, 0.40 mmol), and (S)-2-[2-(3-fluoro-phenyl)-ethyl]-piperazine (0.083 g, 0.40 mmol). Purification via flash chromatography, eluting with a step gradient starting with dichloromethane and going to 97% dichloromethane with 3% 2 M ammonia in methanol, to give the free base of the title compound. The free base is then converted to the succinate salt to give the title compound: Mass Spectrum (m/e): 421(M+1).

### Example 177

### (S)-10-[3-[2-(4-Fluoro-phenyl)-ethyl]-piperazin-1-yl]-2-methyl-4H-3-thia-1,9-diaza-benzo[f]azulene succinate

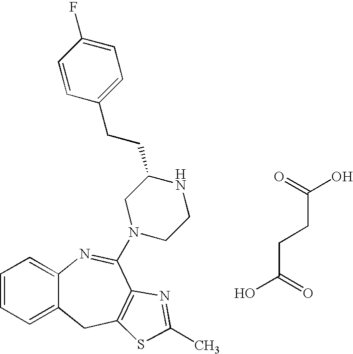

By using a method similar to Example 174, using 2-methyl-4,9-dihydro-3-thia-1,9-diaza-benzo[f]azulene-10-thione (0.081 g, 0.33 mmol), methyl trifluormethanesulfonate (0.081 g, 0.40 mmol) and (S)-2-[2-(4-fluoro-phenyl)-ethyl]-piperazine (0.103 g, 0.40 mmol). Purification via flash chromatography, eluting with a step gradient starting with dichloromethane and going to 97% dichloromethane with 3% 2 M ammonia in methanol, to give the free base of the title compound. The free base is then converted to the succinate salt to give the title compound: Mass Spectrum (m/e): 421 (M+1).

### Example 178

### (S)-10-[3-[2-(4-Fluoro-phenyl)-ethyl]-4-methyl-piperazin-1-yl]-2-methyl-4H-3-thia-1,9-diaza-benzo[f]azulene succinate

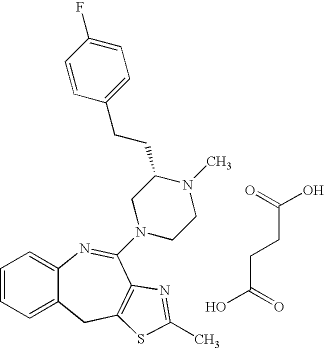

By using a method similar to Example 167, using (S)-10-[3-[2-(4-fluoro-phenyl)-ethyl]-piperazin-1-yl]-2-methyl-4H-3-thia-1,9-diaza-benzo[f]azulene (0.085 g, 0.20 mmol) and purification via flash chromatography, eluting with a step gradient starting with dichloromethane and going to 97% dichloromethane with 3% 2 M ammonia in methanol, gives the free base of the title compound. The free base is then converted to the succinate salt to give the title compound: Exact Mass: Calc. 435.2019; Found 435.2006.

### Example 179

### (S)-10-[3-[2-(3-Fluoro-phenyl)-ethyl]-4-methyl-piperazin-1-yl]-2-methyl-4H-3-thia-1,9-diaza-benzo[f]azulene succinate

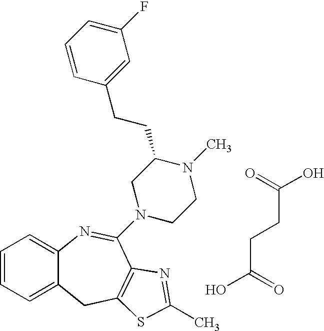

By using a method similar to Example 167, using (S)-10-[3-[2-(3-fluoro-phenyl)-ethyl]-piperazin-1-yl]-2-methyl-4H-3-thia-1,9-diaza-benzo[f]azulene (0.070 g, 0.17 mmol) and purification via flash chromatography, eluting with a step gradient starting with dichloromethane and going to 97% dichloromethane with 3% 2 M ammonia in methanol, gives the free base of the title compound. The free base is converted to the succinate salt to give the title compound: Exact Mass: Calc. 435.2019; Found 435.2023.

### Example 180

### (S)-2-Isopropyl-10-[3-[2-(4-methoxy-phenyl)-ethyl]-piperazin-1-yl]-4H-3-thia-1,9-diaza-benzo[f]azulene hydrochloride

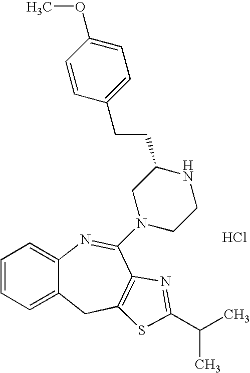

Add 2-isopropyl-4,9-dihydro-3-thia-1,9-diaza-benzo[f]azulen-10-one (0.2 g, 0.77 mmol, to phosphorus oxychloride (6.0 mL)and reflux for 2 hours. Allow the mixture to cool to ambient temperature then concentrate under reduced pressure. Treat the reaction mixture with saturated sodium bicarbonate and extract with dichloromethane. Combine the organic layers, dry over sodium sulfate and concentrate under reduced pressure. Dissolve the crude residue and (S)-2-[2-(4-methoxy-phenyl)-ethyl]-piperazine (0.34 g, 1.54 mmol) in dioxane (10 mL) and pyridine (2.0 mL). Reflux the mixture for 18 hours then cool to ambient temperature. Treat the reaction mixture with ammonium hydroxide and extract with dichloromethane. Combine the organic layers, dry over sodium sulfate and concentrate under reduced pressure. Purification by flash chromatography, eluting with dichloromethane/dichloromethane:methanol:ammonia (94%:6%:0.35 M gradient) gives (S)-2-isopropyl-10-[3-[2-(4-methoxy-phenyl)-ethyl]-piperazin-1-yl]-4H-3-thia-1,9-diaza-benzo[f]azulene. Treating the resulting semisolid in methanol with 1N HCl in ether and concentrating under reduced pressure gives the title compound: mass spectrum (m/e): 461.2 (M+1)of free base.

Using the method of Example 180 gives the following compounds, isolated as the free base except where noted:

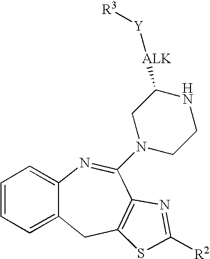

| No. | Alk | Y | R³ | R² | SALT | DATA |
|---|---|---|---|---|---|---|
| 181 | CH₂CH₂ | bond | (4-F)phenyl | isopropyl | succinate | Mass spectrum |
| | | | | | | (m/e): 449.2 |
| 182 | CH₂CH₂ | O | CH₃ | isopropyl | succinate | Mass spectrum |
| | | | | | | (m/e): 385.1 |
| 183 | CH₂CH₂ | bond | (4-OCH₃)phenyl | isobutyl | HCl | Mass spectrum |
| | | | | | | (m/e): 475.1 |
| 184 | CH₂CH₂ | bond | (4-F)-phenyl | isobutyl | HCl | Mass spectrum |
| | | | | | | (m/e): 463.2 |
| 185 | CH₂CH₂ | bond | (4-OCH₃)phenyl | Tert- | HCl | Mass spectrum |
| | | | | butyl | | (m/e): 463.2 |
| 187 | CH₂CH₂ | O | CH₃ | Tert- | HCl | Mass spectrum |
| | | | | butyl | | (m/e): 399.1 |
| 188 | CH₂CH₂ | bond | (3-F)-phenyl | Tert- | HCl | Mass spectrum |
| | | | | butyl | | (m/e): 463.1 |
| 189 | CH₂CH₂ | bond | (4-F)-phenyl | Tert- | HCl | Mass spectrum |
| | | | | butyl | | (m/e): 477.2 |

### Example 190

### (S)-2-Isopropyl-10-[3-[2-(4-methoxy-phenyl)-ethyl]-4-methyl-piperazin-1-yl]-4H-3-thia-1,9-diaza-benzo[f]azulene di-hydrochloride salt

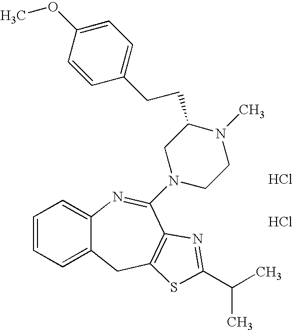

Add sodium triacetoxyborohydride (0.17 g, 0.80 mmol) and formaldehyde (0.045 g, 0.58 mmol, 37%) to a solution of (S)-2-isopropyl-10-[3-[2-(4-methoxy-phenyl)-ethyl]-piperazin-1-yl]-4H-3-thia-1,9-diaza-benzo[f]azulene (0.18 g, 0.39 mmol) in dichloroethane (10 mL) and stir the mixture for two hours. Treat the reaction mixture with saturated sodium bicarbonate and extract with dichloromethane. Combine the organic layers, dry over sodium sulfate and concentrate under reduced pressure. Purification by flash chromatography, eluting with dichloromethane/dichloromethane:methanol:ammonia (94%:6%:0.35 M gradient) gives (S)-2-isopropyl-10-[3-[2-(4-methoxy-phenyl)-ethyl]-4-methyl-piperazin-1-yl]-4H-3-thia-1,9-diaza-benzo[f]azulene. Treating the resulting oil in ethyl acetate with 1 N HCl in ether (0.5 ml) and concentrating under reduced pressure gives the title compound: exact mass spectrum: 475.2542 (M+1).

Using the method of Example 190 gives the following compounds, isolated as the free base except where noted:

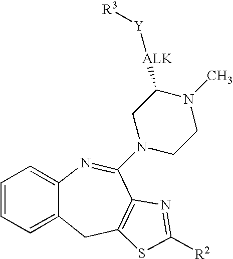

| NO: | Alk | Y | R³ | R² | SALT | DATA |
|---|---|---|---|---|---|---|
| 191 | CH₂CH₂ | bond | (4-F)-phenyl | isopropyl | HCl | Exact Mass |
| | | | | | | spectrum (m/e): |
| | | | | | | 463.2360 |
| 193 | CH₂CH₂ | O | CH₃ | isopropyl | succinate | Mass spectrum |
| | | | | | | (m/e): 399.1 |
| 194 | CH₂CH₂ | bond | (4-OCH₃)-phenyl | isobutyl | HCl | Mass spectrum |
| | | | | | | (m/e): 489.1 |
| 195 | CH₂CH₂ | bond | (4-F)-phenyl | isobutyl | HCl | Exact Mass |
| | | | | | | spectrum (m/e): |
| | | | | | | 466.6615 |
| 196 | CH₂CH₂ | bond | (4-OCH₃)-phenyl | Tertbutyl | HCl | Exact Mass |
| | | | | | | spectrum (m/e): |
| | | | | | | 489.2680 |
| 198 | CH₂CH₂ | O | CH₃ | Tertbutyl | HCl | Exact Mass |
| | | | | | | spectrum (m/e): |
| | | | | | | 413.2350 |
| 199 | CH₂CH₂ | bond | (4-F)-phenyl | Tertbutyl | HCl | Exact Mass |
| | | | | | | spectrum (m/c): |
| | | | | | | 477.2500 |
| 200 | CH₂CH₂ | bond | (4-OCH₃)-phenyl | Isopropyl | Citrate | Mass spectrum |
| | | | | | | (m/e): 489.2680 |
| 201 | CH₂CH₂ | bond | (3-F)-phenyl | Tertbutyl | HCl | Exact Mass |
| | | | | | | spectrum (m/e): |
| | | | | | | 477.2500 |

### Example 220

### (S)-10-[3-(2-Methoxy-ethyl)-piperazin-1-yl]-2-methyl-3,4-dithia-9-aza-benzo[f]azulene dihydrochloride

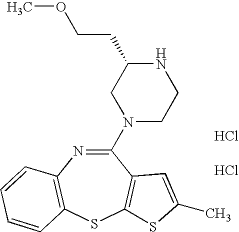

Dissolve 2-methyl-3,4-dithia-9-aza-benzo[f]azulen-10-ylamine (0.33 g, 1.34 mmol) and (S)-methoxyethylpiperazine (0.958 g, 6.64 mmol) in toluene (6.5 ml) and methyl sulfoxide (3.3 ml). Add 10 drops of glacial acetic acid and heat to reflux for a total of 7 days. Add fArther acetic acid (10 drops) every 48 hours. Cool to room temperature then dilute with 2 M NaOH such that pH=9. Extract into ethyl acetate (3×50 ml). Combine organic layers and wash with saturated sodium chloride solution. Separate organic layer, dry over magnesium sulfate, filter and remove solvent by evaporation under reduced pressure. Purify by reverse phase HPLC then add HCl to form salt to give 0.31 g, 63% of the title compound: Mass spectrum 374 M+H for free base.

### Example 222

### (S)-2-Methyl-10-(3-phenethyl-piperazin-1-yl)-3,4-dithia-9-aza-benzo[f]azulene dihydrochloride

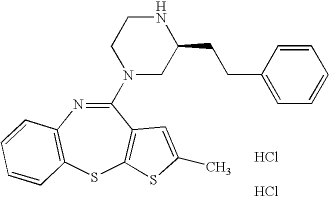

By using a method similar to the method of Example 220, using 2-methyl-3,4-dithia-9-aza-benzo[f]azulen-10-ylamine and (S)-3-phenethyl-piperazine gives the title compound: Mass spectrum 420 M+H for free base.

### Example 224

### 2-R²-10-(4-R¹-(R³⁻-Y-Alk)-piperazin-1-yl)-9H-3,4-dithia-9-azabenzo[f]azulene dibydrochloride

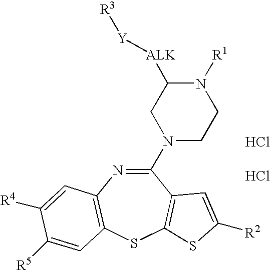

Stir the appropriate thione (1 equiv) in dry dichloromethane (0.5 M) under nitrogen in an ice-bath and treat with methyl trifluoromethanesulfonate (1.5 equiv.). Stir the mixture at 0-5° C. for 5 mins and allow to warn to ambient to give a clear yellow solution. After stining for 1 hour, add the desired piperazine (1.5 equiv) and dry pyridine (1 mmol thione/1.2 mL pyridine) and stir the mixture under nitrogen at 115° C. for 20 hours. Partition the mixture between dilute ammonia solution and ethyl acetate, wash the organic phase with saline solution, dry over magnesium sulfate and remove the solvent. Purify the crude product, initially by flash chromatography on silica gel, eluting with 0-10% 2 M NH₃/MeOH in dichloromethane, and finally by prep-Ic to give the free base which was converted to the dihydrochlcride salt.

### Example 225

### 2-Ethyl-10-[3(S)-(2-methoxyethyl)piperazin-1-yl]-9H-3,4-dithia-9-azabenzo[f]azulene dihydrochloride

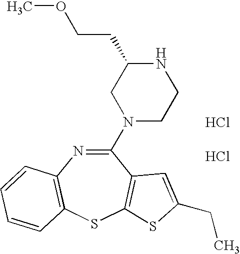

By using a method similar to the method of Example 224, using 2(S)-(methoxyethyl)piperazine and purification by flash chromatography on silica gel eluting with 0-10% 2 M NH₃/MeOH in dichloromethane, and finally by prep-lc to give the title compound. 15% yield, free base (m.s m/e M+1: 387) converted to dihydrochloride salt. m.p. 164-166° C.

### Example 227

### 2-Isopropyl-10-[3(S)-(2-methoxyethyl)piperazin-1-yl]-9H-3,4-dithia-9-azabenzo[f]azulene dihydrochloride

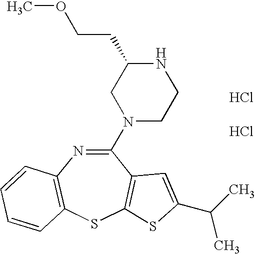

By using a method similar to the method of Example 224, using 2-isopropyl-9H-3,4-dithia-9-azabenzo[f]azulene-10-thione and 2(S)-(methoxyethyl)piperazine and purification, initially by flash chromatography on silica gel eluting with 0-10% 2 M NH₃/MeOH in dichloromethane, and finally by prep-lc to give the free base of the title compound yield: 87%. (m.s. m/e: M+1: 402), converted to the dihydrochloride salt. m.p. 149-152° C.dec.

### Example 228

### 2-Isopropyl-4-[3(S)-3-[2-(3-fluorophenyl)ethyl]-piperazin-1-yl]-9H-3,4-dithia-9-azabenzo[f]azulene dihydrochloride

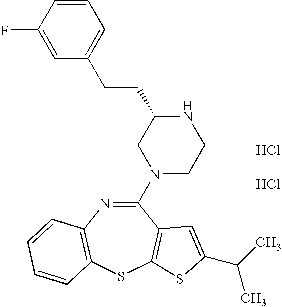

By using a method similar to the method of Example 224, using 2-isopropyl-9H-3,4-dithia-9-azabenzo[f]azulene-10-thione and 2(S)-[2-(3-fluorophenyl)ethyl]piperazine. Purified by flash chromatography on silica gel eluting with 0-10% 2 M NH₃/MeOH in dichloromethane. Free-base yield: 64%. (m.s. m/e M+1: 466), which was converted to the di-hydrochloride salt.

### Example 229

### 2-Isopropyl-4-[3(S)-3-[2-(4-methoxyphenyl)ethyl]-piperazin-1-yl]-9H-3,4-dithia-9-azabenzo[f]azulene dihydrochloride

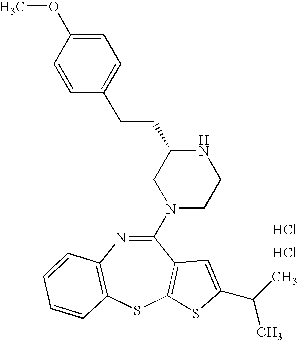

By using a method similar to the method of Example 224, using 2-isopropyl-9H-3,4-dithia-9-azabenzo[f]azulene-10-thione and 2(S)-[2-(4-methoxyphenyl)ethyl]piperazine and purification by flash chromatography on silica gel eluting with 0-10% 2 M NH₃/MeOH in dichloromethane to give the free base, 63% yield which was converted to the dihydrochloride salt;, m.s.m/e (M+1: 478).

### Example 230

### 2-Isopropyl-4-[3(S)-3-[2-(4-methoxyphenyl)ethyl]-4-methyl-piperazin-1-yl]-9H-3,4-dithia-9-azabenzo[f]azulene dihydrochloride

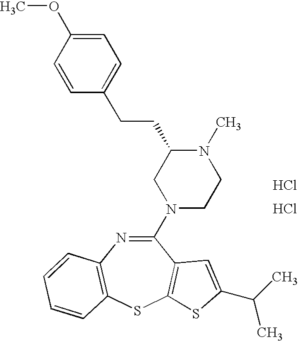

Stir 2-isopropyl-4-[3(S)-3-[2-(3-fluorophenyl)ethyl]-piperazin-1-yl]-9H-3,4-dithia-9-azabenzo[f]azulene (377 mg, 0.79 mmol) in dichloroethane (150 mL) add 37% aqueous formaldehyde solution (0.38 mL, 4.68 mmol), followed by sodium triacetoxyborohydride (1 g, 4.72 mmol). Stir the mixture vigorously for 1 hour, wash with saturated aq. sodium bicarbonate solution and saline, dry over magnesium sulfate and remove the solvent removed to leave the pure free base of the title compound: 322 mg, 83% yield, (m.s. m/e: M+1: 492), which was converted to the dihydrochloride salt.

### Example 231

### 2-Ethyl-10-[3(S)-(2-methoxyethyl)-4-methyl-piperazin-1-yl]-9H-3,4-dithia-9-azabenzo[f]azulene dihydrochloride

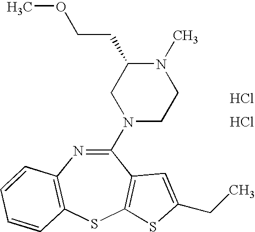

By using a method similar to the method of Example 230 using 2-ethyl-10-[3(S)-(2-methoxyethyl)piperazin-1-yl]-9H-3,4-dithia-9-azabenzo[f]azulene to give the Free base yield: 78%, (m.s. m/e M+1: 402) which was converted to the dihydrochloride salt mp 181-187° C.

### Example 232

### 2-Isopropyl-10-[3(S)-(2-methoxyethyl)-4-methylpiperazin-1-yl]-9H-3,4-dithia-9-azabenzo[f]azulene dihydrochloride

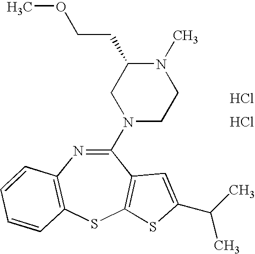

By using a method similar to the method of Example 230, using 2-isopropyl-10-[3(S)-(2-methoxyethyl)piperazin-1-yl]-9H-3,4-dithia-9-azabenzo[f]azulene and purification by flash chromatography on silica gel ejuting with 0-10% 2 M NH₃/MeOH in dichloromethane to give the free-base of the title compound, 43% yield, (m.s. m/e: M+1: 416) which was converted to the di-hydrochloride salt mp 143-147° C. dec.

### Example 233

### 2-Isopropyl-4-[3(S)-3-[2-(3-fluorophenyl)ethyl]-4-methylpiperazin-1-yl]-9H-3,4-dithia-9-azabenzo[f]azulene dihydrochloride

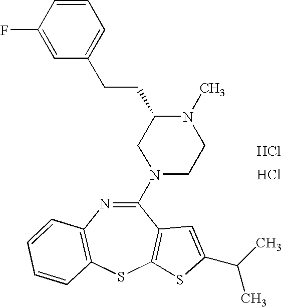

By using a method similar to the method of Example 230, using 2-isopropyl-4-[3(S)-3-[2-(3-fluorophenyl)ethyl]-piperazin-1-yl]-9H-3,4-dithia-9-azabenzo[f]azulene to give the freebase yield: 65%. (m.s. m/e M+1: 480) which was converted to the di-hydrochloride salt.

### Example 234

### (S)-2-Methyl-10-[3-(2-methoxy-ethyl)-piperazin-1-yl]-3,4-dithia-9-aza-benzo[f]azulene dihydrochloride

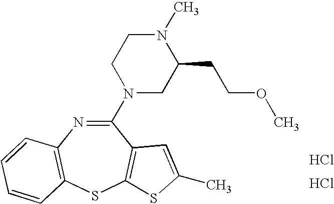

By using a method similar to the method of Example 230 to give the title compound: mass spectrum 388 M+H for free base.

### Example 235

### (S)-2-Methyl-10-(4-methyl-3-phenethyl-piperazin-1-yl)-3,4-dithia-9-aza-benzo[f]azulene dihydrochloride

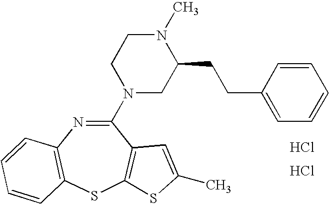

By using a method similar to the method of Example 230 to give the title compound: Mass spectrum 434 M+H for free base.

### Example 237

### (S)-10-[3-[2-(3-Fluoro-phenyl)-ethyl]-piperazin-1-yl]-2-isobutyl-3,4-dithia-9-aza-benzo[f]azulene dihydrochloride

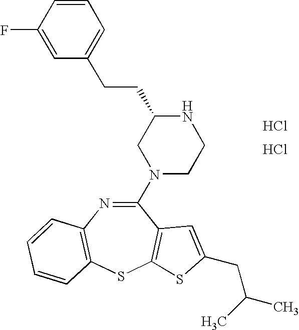

By using a method similar to the method of Example 224, using 2-isobutyl-9H-3,4-dithia-9-aza-benzo[f]azulene-10-thione to give the title compound: mass spectrum 480 M+H for free base.

### Example 238

### (S)-10-[3-[2-(3-Fluoro-phenyl)-ethyl]-4-methyl-piperazinz-1-yl]-2-isobutyl-3,4-dithia-9-aza-benzo[f]azulene dihydrochloride

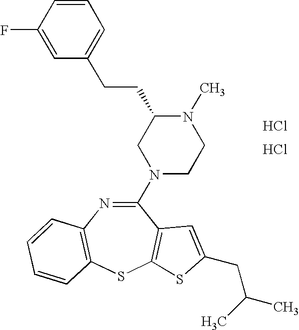

By using a method similar to the method of Example 230 to give the title compound: mass Spectrum; 494 M+H for free base, ¹H NMR: 11.59 (m, 1H), 7.14 (m, 9H), 5.75 (m, 1H), 3.85 (m, 2H), 3.55 (m, 2H), 3.35 (m, 2H), 3.14 (m, 1H), 2.87 (m, 2H), 2.80 (m, 1H), 2.62 (m, 3H), 2.31 (m, 1H), 1.91 (m, 2H), 1.80 (m, 1H), 1.23 (m, 1H), 0.87 (m, 6H).

### Example 240

### (S)-2-Propyl-10-[3-(2-methoxy-ethyl)-piperazin-1-yl]-3,4-dithia-9-aza-benzo[f]azulene dihydrochloride

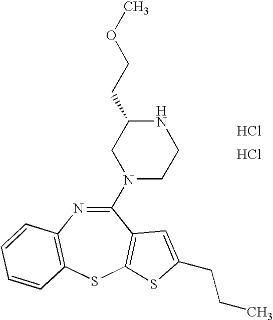

By using a method similar to the method of Example 224, using 2-Propyl-9H-3,4-dithia-9-aza-benzo[f]azulene-10-thione: mass spectrum 402 M+H for free base.

### Example 241

### (S)-2-Propyl-10-[3-(2-methoxy-ethyl)-4-methyl-piperazin-1-yl]-3,4-dithia-9-aza-benzo[f]azulene dihydrochloride

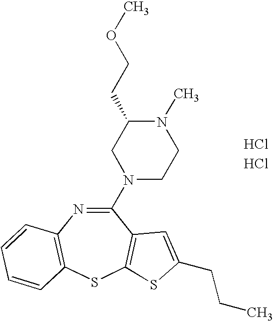

By using a method similar to the method of Example 230 gives the title compound: mass spectrum 416 M+H for free base. ¹H NMR: 7.35 (m, 5H), 3.56 (m), 3.16 (m), 2.83 (m), 2.70 (m), 2.49 (s, 3H), 1.81 (m, 2H), 1.57 (m, 2H), 0.87 (m, 3H).

### Example 242

### (S)-2-Propyl-10-[3-[2-(4-methoxy-phenyl)-ethyl]-piperazin-1-yl]-3,4-dithia-9-aza-benzo[f]azulene dihydrochloride

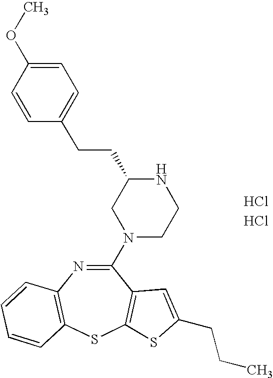

By using a method similar to the method of Example 224, using 2-Propyl-9H-3,4-dithia-9-aza-benzo[f]azulene-10-thione to give the title compound: mass spectrum 478 M+H for free base.

### Example 243

### (S)-2-Propyl-10-[3-[2-(4-methoxy-phenyl)-ethyl]-4-methyl-piperazin-1-yl]-3,4-dithia-9-aza-benzo[f]azulene dihydrochloride

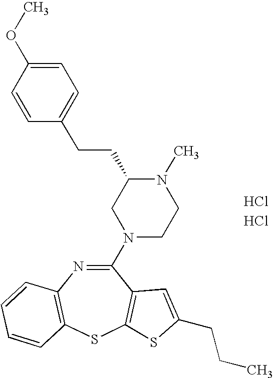

By using a method similar to the method of Example 230 to give the title compound: mass spectrum 492 M+H for free base; ¹H NMR: 11.43 (m, 1H), 7.38 (m, 2H), 7.08 (m, 5H), 6.85 (m, 2H), 5.75 (m, 1H), 3.74 (m, 3H), 3.50 (m, 3H), 2.78 (m, 6H), 2.50 (m, 3H), 2.27 (m, 1H), 1.91 (m, 2H), 1.57 (m, 2H), 1.23 (m, 1H), 0.88 (m, 3H).

### Example 244

### (S)-10-[3-[2-(3-Fluoro-phenyl)-ethyl]-piperazin-1-yl]-2-propyl-3,4-dithia-9-aza-benzo[f]azulene dihydrochloride

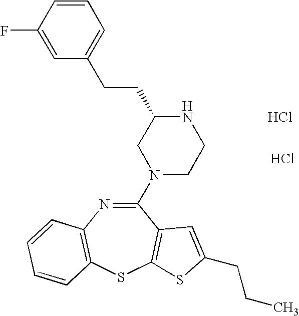

By using a method similar to the method of Example 224, using 2-Propyl-9H-3,4-dithia-9-aza-benzo[f]azulene-10-thione to obtain the title compound: mass spectrum 466 M+H for free base.

### Example 245

### (S)-10-[3-[2-(3-Fluoro-phenyl)-ethyl]-4-methyl-piperazin-1-yl]-2-propyl-3,4-dithia-9-aza-benzo[f]azulene dihydrocbloride

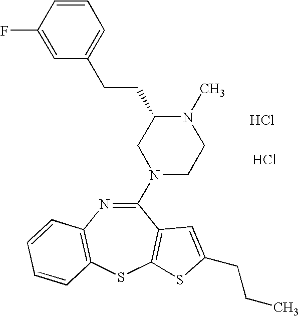

By using a method similar to the method of Example 230 to give the title compound: mass spectrum 480 M+H for free base. ¹H NMR: 11.84 (bs, 1H), 7.12 (m, 9H), 5.76 (s, 1H), 3.80 (m, 1H), 3.54 (m, 2H), 3.38 (m, 1H), 3.16 (m, 4H), 3.79 (m, 4H), 2.34 (m, 3H), 1.98 (m, 1H), 1.59 (m, 2H), 0.87 (m, 6H).

### Example 255

### 11-(4-Methyl-3-(S)-phenethyl-piperazin-1-yl)-dibenzo[b,f][1,4]thiazepine

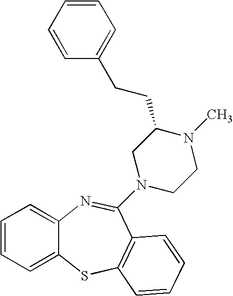

Add aqueous 37% formaldehyde (1.1 equiv) to a solution of 11-(3-(S)-Phenethyl-piperazin-1-yl)-dibenzo[b,f][1,4]thiazepine (400 mg) (1 equiv,) in dichloroethane (1.6 M). Stir the mixture 2 minutes and add sodium triacetoxyborohydride (1.5 equiv). Stir the suspension for 30 minutes and quench with a saturated aqueous solution of sodium bicarbonate. Extract the aqueous phase 3 times with dichloromethane and combine the organic phases, dry over magnesium sulfate, filter and concentrate. Purify the residue via chromatography on silica gel (methylene chloride/methanol (90:10)) to provide the title compound as a white solid (297 mg, 72%): mp 44-54° C.; ¹H NMR (CDCl₃) δ1.99-1.55 (m, 2H), 2.33 (s, 3H), 3.04-2.07 (m, 5.5H), 3.07 (t, 1H), 3.35 (t, 0.5H), 6.87 (ddd, 1H), 7.07 (dd, 1H), 7.35-7.05 (m, 9H), 7.38 (dt, 1H), 7.52-7.48 (m, 1H); MS (APCI) m/z (rel intensity) 414.4 (100).

### Example 256

### 11-(3-(R)-Benzyl-piperazin-1-yl)-dibenzo[b,f][1,4]thiazepine

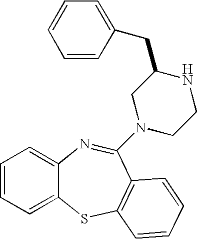

Combine 11-chloro-dibenzo[b,f][1,4]thiazepine (700 mg), 2-(R)-benzyl-piperazine (1.0 g) and toluene (10 mL), heat to reflux for 19 hours, then cool down to ambient temperature. Concentrate and purify by flash chromatography (dichloromethane then gradient of methanol 3-10%) to give the title compound as a white solid (734 mg, 74%): mp 56-69° C.; ¹H NMR (CDCl₃) δ2.58 (dd, 1H), 3.18-2.60 (m, 6H), 6.88 (dt, 1H), 7.07 (dd, 1H), 7.34-7.14 (m, 9H), 7.39 (dd, 1H), 7.53-7.47 (m, 1H); MS (APCI) m/z (rel intensity) 386.4 (100).

### Example 257

### 11-(3-(R)-Benzyl-4-methyl-piperazin-1-yl)-dibenzo[b,f][1,4]thiazepine

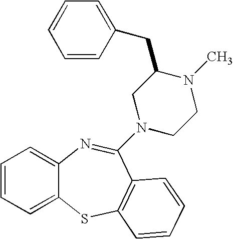

By the reductive methylation method described for Example 255, 11-(3-(R)-benzyl-piperazin-1-yl)-dibenzo[b,f][1,4]thiazepine (385 mg) affords the title compound as a white solid (356 mg, 89%): mp 46-63° C.; ¹H NMR (CDCl₃) δ 2.24-2.57 (m, 3H), 2.50 (s, 3H), 2.79 (dt, 1H), 2.83-2.99 (m, 1H), 3.07-3.17 (m, 2H), 3.32 (ddd, 1H), 6.82-6.89 (m, 1H), 6.94-7.25 (m, 10 H), 7.36 (dt, 1H), 7.44-7.37 (m, 1H); MS (APCI) m/z (rel intensity) 400.3 (100).

### Example 258

### 11-(3-(S)-Phenethyl-piperazin-1-yl)-dibenzo[b,f][1,4]thiazepine

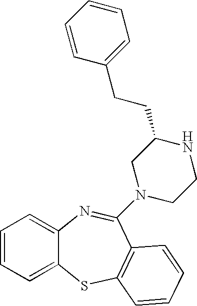

By the coupling procedure described for Example 256, 11-chloro-dibenzo[b,f][1,4]thiazepine (430 mg) and 2-(S)-phenethyl-piperazine (1.0 g) afford the title compound as a white solid (632 mg, 91%): mp 48-59° C.; ¹H NMR (CDCl₃) δ1.80-1.62 (m, 3H), 3.13-2.48 (m, 7H), 6.87 (dt, 1H), 7.07 (dd, 1H), 7.35-7.12 (m, 9H), 7.38 (dt, 1H), 7.53-7.48 (m, 1H); MS (APCI) m/z (rel intensity) 400.4 (100).

### Example 259

### 11-(3-(S)-Benzyl-piperazin-1-yl)-dibenzo[b,f][1,4]thiazepine

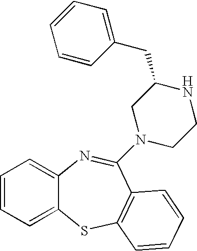

By the coupling procedure described for Example 256, 11 -chloro-dibenzo[b,f][1,4]thiazepine (500 mg) and 2-(S)-benzyl-piperazine (700 mg) afford the title compound as a white solid (692 mg, 89%): mp 54-71° C.; ¹H NMR (CDCl₃) δ 2.56 (dd, 1H), 3.18-2.60 (m, 6H), 6.86 (dt, 1H), 7.07-7.03 (m, 1H), 7.31-7.12 (m, 9H), 7.39-7.35 (m, 1H), 7.49-7.46 (m, 1H); MS (APCI) m/z (rel intensity) 386.4 (100).

### Example 260

### 11 -(3-(S)-Benzyl-4-methyl-piperazin-1-yl)-dibenzo[b,f][1,4]thiazepine

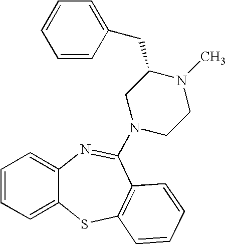

By the reductive methylation method described for Example 255, 11-(3-(S)-benzyl-piperazin-1-yl)-dibenzo[b,f][1,4]thiazepine (335 mg) afford the title compound as a white solid (288 mg, 83%): mp 48-64° C.; ¹H NMR (CDCl₃) δ 2.51-2.29 (m, 3H), 2.49 (s, 3H), 3.15-2.72 (m, 4H), 3.32 (t, 1H), 6.91-6.85 (m, 1H), 7.00 (dd, 1H), 7.29-7.05 (m, 9H), 7.37 (dt, 1H), 7.47-7.39 (m, 1H); MS (APCI) m/z (rel intensity) 400.3 (100).

### Example 336

### 10-[3-[2(S)-(3-Fluoro-phenyl)-ethyl]-4-methyl-piperazin-1-yl]-3,4-dithia-1,9-diaza-benzo[f]azulene

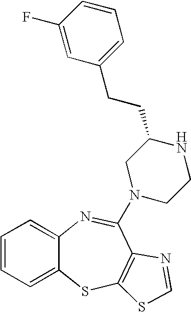

Stir 9H-3,4-dithia-benzo[f]azulen-10-thione (1 equiv) in dry dichloromethane (1 mmol thione to approx 7 mL of solvent) under nitrogen in an ice-bath and treat with methyl trifluoromethanesulfonate (1.7 equiv). Stir the mixture at 0-5° C. for 5 mins and allow to warm to ambient to give a clear yellow solution. After stirring for 3 hours, remove the solvent under reduced pressure. Add 2(S)-[2-(3-fluoro-phenyl)ethyl]piperazine (approx 6.3 equiv) and dry pyridine (2.9 mL per equiv of thione) and stir the mixture under nitrogen at 115° C. for 20 hours. Cool the reaction to room temperature and concentrate under reduced pressure. Purify the crude product, by chromatography on silica gel, eluting with 0-3% 2 M NH₃/MeOH in dichloromethane to give the title compound: NMR (CDCl₃) δ 1.95 (2H, broad s), 2.70 (2H,broad s), 3.00-3.40 (4H, m), 4.00-4.70 (3H, m), 6.75-7.30 (8H, m), 8.70 (1H, s); Mass Spectrum (m/e): 425 (M+1).

### Example 337

### 2-Ethyl-10-[3-[2(S)(4-methoxy-phenyl)-ethyl]-piperazin-1-yl]-3,4-dithia-1,9-diaza-benzo[f]azulene

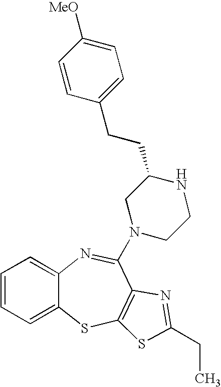

By using a method similar to the method of Example 336 and 2-ethyl-9H-3,4-dithia-benzo[f]azulen-10-thione and 2(S)-[2-(4-methoxyphenyl)ethyl]piperazine, the title compound 2-ethyl-10-[3-[2(S)(4-methoxy-phenyl)-ethyl]-piperazin-1-yl]-3,4-dithia-1,9-diaza-benzo[f]azulene, is prepared. Mass Spectrum (m/e): 464 (M+1).

### Example 338

### 2-Isopropyl-10-[3-[2(S)(4-methoxy-phenyl)-ethyl]-piperazin-1-yl]-3,4-dithia-1,9-diaza-benzo[f]azulene

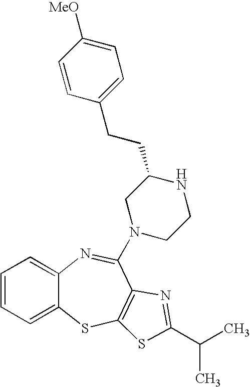

By using a method similar to the method of Example 336 and 2-isopropyl-9H-3,4-dithia-benzo[f]azulen-10-thione and 2(S)-[2-(4-methoxyphenyl)ethyl]piperazine, the title compound 2-isopropyl-10-[3-[2(S)(4-methoxy-phenyl)-ethyl]-piperazin-1-yl]-3,4-dithia-1,9-diaza-benzo[f]azulene, is prepared. Mass Spectrum (m/e): 478 (M+1).

### Example 339

### 2-Ethyl-10-[3-[2(S)(3-fluoro-phenyl)-ethyl]-piperazin-1-yl]-3,4-dithia-1,9-diaza-benzo[f]azulene

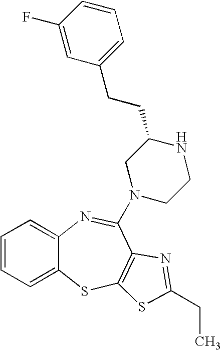

By using a method similar to the method of Example 336 and 2-ethyl-9H-3,4-dithia-benzo[f]azulen-10-thione and 2(S)-[2-(3-fluorophenyl)ethyl]piperazin, the title compound 2-ethyl-10-[3-[2(S)(3-fluoro-phenyl)-ethyl]-piperazin-1-yl]-3,4-dithia-1,9-diaza-benzo[f]azulene, is prepared. Mass Spectrum (m/e): 453 (M+1).

### Example 340

### 2-Isopropyl-10-[3-[2(S)(3-fluoro-phenyl)-ethyl]-piperazin-1-yl]-3,4-dithia-1,9-diaza-benzo[f]azulene

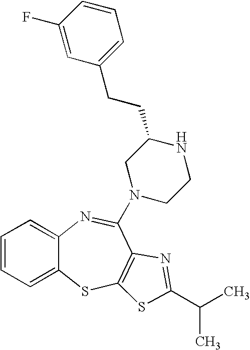

By using a method similar to the method of Example 336 and 2-isopropyl-9H-3,4-dithia-benzo[f]azulen-10-thione and 2(S)-[2-(3-fluorophenyl)ethyl]piperazine, the title compound 2-isopropyl-10-[3-[2(S)(3-fluoro-phenyl)-ethyl]-piperazin-1-yl]-3,4-dithia-1,9-diaza-benzo[f]azulene, is prepared. Mass Spectrum (m/e): 467 (M+1).

### Example 341

### 10-[3-(2(S)-Methoxy-ethyl)-piperazin-1-yl]-3,4-dithia-1,9-diaza-benzo[f]azulene

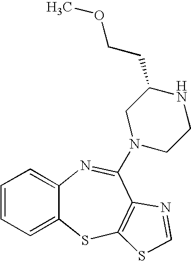

By using a method similar to the method of Example 330 and 9H-3,4-dithia-1,9-diaza-benzo[f]azulen-10-thione and 2(S)-[2-(4-methoxyphenyl)ethyl]piperazine, the title compound 10-[3-(2(S)-methoxy-ethyl)-piperazin-1-yl]-3,4-dithia-1,9-diaza-benzo[f]azulene, is prepared ¹H NMR (CDCl₃) δ 1.70 (2H, m), 2.50 (2H, broad s), 2.75-3.20 (4H, m), 3.30 (3H, s), 3.50 (2H,m), 4.10 (1H, broad s), 6.90 (1H, t), 7.10 (1H, d), 7.20 (1H, t), 7.30 (1H, d), 8.80 (1H, s). Converted to the hydrochloride salt. Mp 184-186° C. Mass spectrum (m/e) 361 (M+1).

### Example 342

### 2-Ethyl-10-[3-[2(S)(4-methoxy-phenyl)-ethyl]-4-methyl-piperazin-1-yl]-3,4-dithia-1,9-diaza-benzo[f]azulene hydrochloride

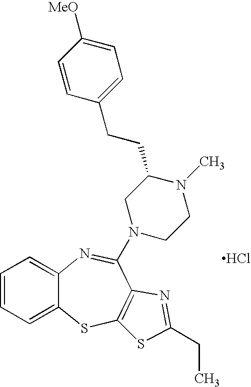

Add sodium triacetoxyborohydride (0.220 g, 1.04 mmol) and formaldehyde (1 mL, 7% solution) to a solution of 2-ethyl-10-[3-[2(S)(4-methoxy-phenyl)-ethyl]-piperazin-1-yl]-3,4-dithia-1,9-diaza-benzo[f]azulene in dichloroethane (150 mL) and stir the mixture for two hours. Treat the reaction mixture with saturated sodium bicarbonate and extract with dichloromethane. Combine the organic .layers, dry over sodium sulfate and concentrate under reduced pressure. Purification by flash chromatography, eluting with DCM: 3% MeOH) gives free base (0.375 g). Treating the resulting oil in with 2 M HCl in diethylether gives the title compound (m.p. 129-130° C.). Mass Spectrum (m/e): 479 (M+1). ¹H NMR (CD₃CN) δ 1.35 (3H, t), 1.6-2.0 (3H, broad) 2.0-2.4 (3H, broad), 2.86 (3H,s), 3.03 (2H, broad), 3.2-3.7 (3H, broad), 3.79 (3H,s), 3.9-4.4 (2H, broad), 6.87 (2H, broad), 7.1-7.8 (6H broad), 12.94 (1H, broad)

### Example 343

### 2-Ethyl-10-[3-[2(S)(3-fluoro-phenyl)-ethyl]-4-methyl-piperazin-1-yl]-3,4-dithia-1,9-diaza-benzo[f]azulene hydrochloride

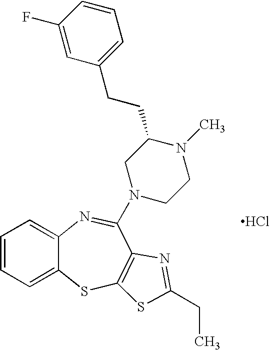

By using a method similar to the method of Example 342 and 2-ethyl-10-[3-[2(S)(3-fluoro-phenyl)-ethyl]-piperazin-1-yl]-3,4-dithia-1,9-diaza-benzo[f]azulene, the title compound 2-Ethyl-10-[3-[2(S)(3-fluoro-phenyl)-ethyl]-4-methyl-piperazin-1-yl]-3,4-dithia-1,9-diaza-benzo[f]azulene hydrochloride, is prepared. Melting point 133-135° C. Mass Spectrum (m/e): 467 (M+1) ¹H NMR (CD₃CN) δ 1.13 (3H, t), 1.6-2.0 (3H, broad) 2.0-2.4 (3H, broad), 2.86 (3H,s), 3.03 (2H, broad), 3.2-3.7 (3H, broad), 3.79 (3H,s), 3.9-4.4 (2H, broad), 6.8 (2H, broad), 6.94 (1H broad), 7.1-7.2 (2H broad), 7.3-7.4 (2H, broad), 7.46 (1H, broad), 13.07 (1H, broad).

### Example 344

### 2-Isopropyl-10-[3-[2(S)(4-methoxy-phenyl)-ethyl]-4-methyl-piperazin-1-yl]-3,4-dithia-1,9-diaza-benzo[f]azulene succinate

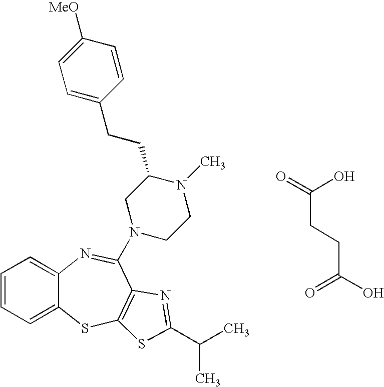

By using a method similar to the method of Example 342 and 2-isopropyl-10-[3-[2(S)(4-methoxy-phenyl)-ethyl]-piperazin-1-yl]-3,4-dithia-1,9-diaza-benzo[f]azulene the free base of the title compound 2-isopropyl-10-[3-[2(S)(4-methoxy-phenyl)-ethyl]-4-methyl-piperazin-1-yl]-3,4-dithia-1,9-diaza-benzo[f]azulene, is prepared. The succinate salt is prepared by dissolving the free base in ethanol, adding 1 equivalent of succinic acid and removing the solvent under reduced pressure. Mass Spectrum (m/e): 493 (M+1) ¹H NMR (CD₃CN) δ 1.25 (6H, d), 1.6-2.0 (3H, broad) 2.0-2.4 (3H, broad), 2.3 (4H, broad), 2.86 (3H,s), 3.15 (1H, broad), 3.2-3.7 (3H, broad), 3.79 (3H,s), 3.9-4.4 (2H, broad), 6.87 (2H, broad), 7.1-7.8 (6H broad).

### Example 345

### 10-[3-[2(S)-(3-Fluoro-phenol)-ethyl]-4-methyl-piperazin-1-yl]-3,4-dithia-1,9-diaza-benzo[f]azulene

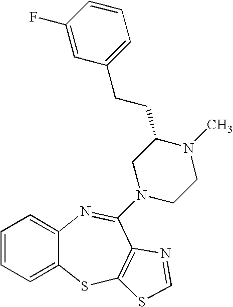

By using a method similar to the method of Example 342 and 10-[3-[2(S)-(3-fluoro-phenyl)-ethyl]-piperazin-1-yl]-3,4-dithia-1,9-diaza-benzo[f]azulene, the title compound 10-[3-[2(S)-(3-fluoro-phenyl)-ethyl]-4-methyl-piperazin-1-yl]-3,4-dithia-1,9-diaza-benzo[f]azulene, is prepared. Mass Spectrum (m/e): 439 (M+1).

### Example 346

### 10-[3-(2(S)-Methoxy-ethyl)-4-methyl-piperazin-1-yl]-3,4-dithia-1.9-diaza-benzo[f]azulene

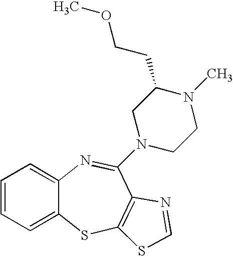

By using a method similar to the method of Example 342 10-[3-(2(S)-methoxy-ethyl)-piperazin-1-yl]-3,4-dithia-1,9-diaza-benzo[f]azulene, the title compound 10-[3-(2(S)-methoxy-ethyl)-4-methyl-piperazin-1-yl]-3,4-dithia-1,9-diaza-benzo[f]azulene, is prepared ¹H NMR (CDCl₃) δ 1.80 (1H, m), 1.95 (1H, m), 2.35 (3H, s), 2.20-2.55 (3H,m), 2.75-3.50 (5H, m), 3.75 (3H, s), 4.00 (1H, broad s), 6.95 (1H, t), 7.10 (1H, d), 7.20 (1H, t), 7.30 (1H, d), 8.65 (1H, s). Converted to the hydrochloride salt. Mp 146-148. Mass Spectrum (ni/e): 375 (M+1).

### Example 350

### 1-(10-[3-[2(S)-(3-Fluoro-phenyl)-ethyl]-4-methyl-piperazin-1-yl]-3,4-dithia-1,9-diaza-benzo[f]azulen-2-yl)-ethanone dihydrochloride

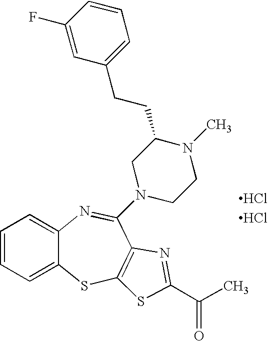

Add LDA (2 equiv, 2 M solution in hexanes) dropwise to a cold solution (−78° C.) of 10-[3-[2(S)-(3-fluoro-phenyl)-ethyl]-4-methyl-piperazin-1-yl]-3,4-dithia-1,9-diaza-benzo[f]azulene (1 equiv) in dry diethylether (1 mmol starting material to 4 mL solvent) under a nitrogen atmosphere with stirring. After 30 min, add 4-acetyl morpholine (1.3 equiv) at −78° C. with stirring. After a further 15 min warm the reaction mixture slowly to −30° C. and then to −5° C. After 2 h quench the reaction by addition of saturated aqueous ammonium chloride solution and ethyl acetate. Separate the layers and wash the organic extract with brine, dry over magnesium sulfate, and concentrate under reduced pressure. Purify the residue by chromatography on silica eluting with MeOH:DCM (1:9) to give the desired product. Further purification by SCX-ion exchange chromatography might be necessary: ¹H NMR (CDCl₃) δ 1.85 (4H, m), 2.2-3.6 (7H,m), 2.35 (3H, s), 2.65 (3H, m), 6.8-7.35 (8H, m). Converted to dihydrochloride salt. Mp 130-132. Mass Spectrum (m/e): 481 (M+1)

### Example 351

### 1-[10-[3-(2(S)-Methoxy-ethyl)-4-methyl-piperazin-1-yl]-3,4-dithia-1,9-diaza-benzo[f]azulen-2-yl]-ethanone dihydrochloride

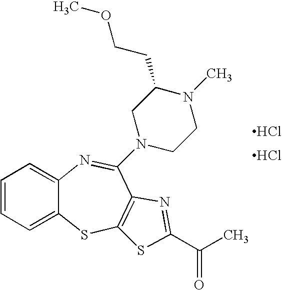

By using a method similar to the method of Example 350 and 10-[3-(2(S)-methoxy-ethyl)-piperazin-1-yl]-3,4-dithia-1,9-diaza-benzo[f]azulene, 1-[10-[3-(2(S)-methoxy-ethyl)-4methyl-piperazin-1-yl]-3,4-dithia-1,9-diaza-benzo[f]azulen-2-yl]-ethanone is prepared. NMR (CDCl₃) δ 1.6-3.6 (11H,m), 2.35 (3H, s), 2.60 (3H, s), 3.20 (3H, s), 6.95 (1H, t), 7.05 (1H, d), 7.20 (1H, t), 7.30 (1H, d). Converted to dihydrochloride salt Mp 138-140° C. Mass Spectrum (m/e): 417 (M+1).

Receptor Binding Assays

### Serotonin 5-HT₆ and Dopamine D₂ binding Assay Protocol

The assay buffers used are 50 mM Tris-HCl pH 7.4, 120 mM NaCl, 5 mM KCl, 5 mM MgCl₂, 1 mM EDTA for the Dopamine D₂s receptor binding assay. The radioligand used is [¹²⁵I]iodospiperone from New England Nuclear Cat # NEX284-2200 Ci/mmole. The membranes used are from Receptor Biology (now owned by NEN), Cat # RBHD2CM for the D₂ receptor.

Compounds are obtained as 10 mM stocks in 100% DMSO. They are diluted to 1 mM in 100% DMSO by adding 180 μL DMSO to 20 μL of stock in 96 well plates using a multidrop. The 1 mM stocks are then diluted to make an 11 point concentration range from 125 μM down to 1.25 nM in half log increments using 10% DMSO as diluent. This. is done using a TECAN robot. The final DMSO at this stage is 10 -21.25% DMSO

The radioligand is diluted in assay buffer to provide 0.1 nM for the D₂ assay. Each vial of membranes is diluted up to 92 mL in assay buffer. The final assay volume is 250 μL consisting of 210 μl of diluted membranes, 20 μL of compound or 10% DMSO for total binding, and 20 μL of diluted radioligand. The compounds are transferred from drug dilution plates into coming 96 well assay plates using a 96 well Multimek pipettor. Radioligand and membranes are added to assay plates using multidrop pipettors. Non-specific binding is determined in wells containing a final concentration of 5 μM haloperidol. The final drug concentration range in half logs is from 10 μM down to 0.1 nM. The final DMSO in the assay is 1-1.7%.

After addition of drug, membrane, and ligand, the plates are incubated for 2 hours at room temperature. During this time 96 well Millipore filter plates (MAFBNOB50) are soaked for a least 30 minutes with 200 μL per well of 0.5% polyethyleneimine.

The 0.5% PEI is removed from filterplate wells using a TiterTek MAP aspirator and 200 μL of the incubation mixture is transferred from the incubation plate to the filterplate after mixing. This transfer is done using the 96 tip Mutimek pipettor. After transfer to the filterplate filterplates are extracted and ished twice with 220 AIL per well of cold buffer on the MAP aspirator. The peel away bottoms are removed from the filterplates and 60 μL per well of microscint 20 scintillation fluid is added per well using a multidrop. Plates are placed into suitable holders and are left at room temperature for 3 hours and are counted for ³H in either a Wallac Microbeta counter or on a Packard Topcount.

### [¹²⁵I]DOI SPA Binding to Rhesus 5-HT_{2A} Receptors Protocol

Incubations are performed in a total volume of 200 μl in 96 well assay plates. 50 μL [¹²⁵I]DOI (NEN, 2200 Ci/mmol, final concentration=0.075 nM) is added to 50 μL of test compounds dissolved in water (±DMSO and /or glacial acetic acid). 50 μL Wheat Germ Agglutinin (WGA) SPA beads, at 1 mg/well, (Amersham Life Sciences) in assay buffer (67 mM Tris-HCl pH 7.4, 13 mM MgCl₂, 0.67 mM EDTA) are then added. Membrane homogenate from cells expressing rhesus 5-HT_{2A} receptors, approximately 0.9 million cells/well, is added last. The plates are covered with sealing tape (FasCal) and allowed to incubate at room temperature for 2 hours. The plates are then centrifuged: at approximately 200×g for 10 minutes at room temperature. The amount of ¹²⁵I-DOI bound to the membranes, i.e. proximate to the WGA SPA beads, is then determined using a Wallac MicroBeta Trilux Scintillation Counter (Wallac, Inc.).

### Pharmaceutical Formulations

Capsule

A pulvule formulation is prepared by blending the active with silicone starch, and filling it into hard gelatin capsules.

| | Per 300 mg capsule | |
|---|---|---|
| Compound of formula (I) | 5.0 | mg |
| Silicone | 2.9 | mg |
| Starch flowable | 292.1 | mg |

Tablet

A tablet formulation is made by granulateing the active with appropriate diluent, lubricant, disintegrant and binder and compressing.

| | Per 300 mg tablet | |
|---|---|---|
| Compound of formula (I) | 10.0 | mg |
| Magnesium stearate | 0.9 | mg |
| Microcrystalline cellulose | 75.0 | mg |
| Povidone | 15.0 | mg |
| Starch, directly compressible | 199.1 | mg |

Injection

An aqueous injection of active is prepared as a freeze-dried plug, for reconstitution in a suitable, sterile diluent before use (to a total volume of 10 ml).

| | |
|---|---|
| Compound of formula (I) | 20.0 mg |
| Mannitol | 20.0 mg |
| N Hydrochloric acid and/or N sodium | |
| hydroxide to adjust pH to 5-5.5. | |

Controlled Release Injection

A controlled release injection for intramuscular injection is formed from a sterile suspension of micronised active in an oleaginous vehicle.

| | | |
|---|---|---|
| Compound of formula (I) | 65.0 | mg |
| Aluminium stearate | 0.04 | mg |
| Sesame oil | 2 | ml |

1. A compound of Formula (I) 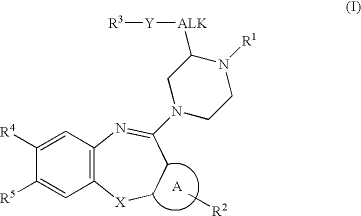
wherein: 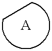
is an optionally benzo-fused five or six member aromatic ring having zero to three hetero atoms independently selected from N, O, and S;
R¹ is hydrogen, (C₁₋₆) fluoroalkyl, (C₃₋₆) cycloalkyl, or (C₁₋₄) alkyl, wherein the (C₁₋₄) alkyl is unsubstituted or substituted with hydroxy, methoxy, ethoxy, OCH₂CH₂OH, —CN, imidazolidin-2-one, phenyl, or tetrazole wherein tetrazole is unsubstituted or substituted with (C₁₋₄) alkyl;
R² is H, halogen, (C₁₋₆) fluoroalkyl, (C₃₋₆) cycloalkyl, OR⁶, SR⁶, NO₂, CN, COR⁶, C(O)OR⁶, C(OH)R⁶, CONR⁷R⁸, phenyl or (C₁₋₆) alkyl, wherein the (C₁₋₆) alkyl is unsubstituted or substituted with a hydroxy;
R³ is hydrogen, (C₁₋₆)fluoroalkyl, (C₃₋₆) cycloalkyl, (C₂₋₆) alkenyl, phenyl, monocyclic heteroaromatic, bicyclic heteroaromatic, or (C₁₋₄)alkyl wherein (C₁₋₄) alkyl is unsubstituted or substituted with a phenyl;
R⁴ and R⁵ are independently selected from hydrogen, halogen, (C₁₋₆) alkyl, (C₁₋₆) fluoroalkyl, OR⁹, SR⁹, NO₂, CN, or COR⁹;
R⁶ is hydrogen, (C₁₋₆) fluoroalkyl, or (C₁₋₆) alkyl;
R⁷ and R⁸ are independently hydrogen, or (C₁₋₆) alkyl;
R⁹ is hydrogen, (C₁₋₆) fluoroalkyl, (C₁₋₆) alkyl;
Alk is (C₁₋₄) alkylene unsubstituted or substituted with a hydroxy;
Y is oxygen, sulfur, S═O, SO₂, or a bond;
X is CH₂, C═O, S, O, or SO₂;
Z is hydrogen, halogen, (C₁₋₆) alkyl, (C₁₋₆)fluoroalkyl, —OH, (C₁₋₆) alkoxy, (C₁₋₆) fluoroalkoxy, (C₁₋₆) alkylthio, (C₁₋₆) acyl, (C₁₋₄)alkylsulfonyl, —OCF₃, —NO₂, —CN, carboxamido which may be substituted on the nitrogen by one or two (C₁₋₄) alkyl groups, and —NH₂ in which one of the hydrogens may be replaced by a (C₁₋₄) alkyl group and the other hydrogen may be replaced by either a (C₁₋₄) alkyl group, a (C₁₋₆) acyl group, or a (C₁₋₄) alkylsulfonyl group;
the phenyl of R¹, R² or R³ is independently unsubstituted or substituted with one to three substituents independently selected from Z;
the monocyclic heteroaromatic of R³is unsubstituted or substituted with one to three substituents independently selected from Z;
the bicyclic heteroaromatic of R³ is unsubstituted or substituted with one to three substituents independently selected from Z;
and salts, solvates, and crystal forms thereof.

2. The compound of claim 1 wherein:
R¹ is hydrogen, (C₁₋₄) alkyl, wherein the (C₁₋₄) alkyl is unsubstituted or substituted with hydroxy, ethoxy, —OCH₂CH₂OH, —CN, imidazolidin-2-one, phenyl, or tetrazole wheren tetrazole is substituted with (C₁₋₄) alkyl;
R² is H, halogen, (C₁₋₆)fluoroalkyl, COR⁶, or (C₁₋₆) alkyl, wherein the (C₁₋₆) alkyl is unsubstituted or substituted with hydroxy;
R³is hydrogen, (C₁₋₄) alkenyl, or phenyl;
R⁴ and R⁵ are independently selected from hydrogen, halogen, (C₁₋₆) alkyl, (C₁₋₆) fluoroalkyl, OR⁹, or NO₂;
R⁶ is hydrogen, or (C₁₋₆) alkyl;
R⁹ is hydrogen, or (C₁₋₆) alkyl; and
X is CH₂, S, or O;
Alk is (C₁₋₄)alkylene; and
Y is O or a bond.

3. The compound of claim 1 wherein 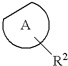
is selected from the group consisting of:

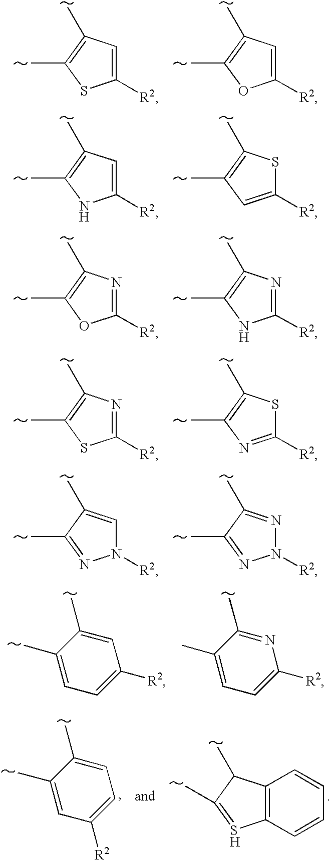

4. The compound of claim 1, wherein

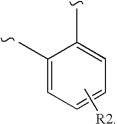

5. The compound of claim 4, wherein:
R¹ is hydrogen or (C₁₋₄) alkyl;
R² is H, (C₁₋₆)fluoroalkyl, (C₁₋₆) alkyl;
R³ is hydrogen, (C₁₋₄) alkyl, or phenyl;
R⁴ and R⁵ are independently selected from hydrogen, halogen, (C₁₋₆) alkyl, (C₁₋₆) fluoroalkyl;
Alk is (C₁₋₄) alkylene;
Y is O or a bond; and
X is S.

6. The compound of claim 1, wherein

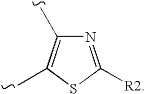

7. The compound of claim 6, wherein:
R¹ is hydrogen, (C₁₋₄) alkyl, wherein the (C₁₋₄) alkyl is unsubstituted or substituted with hydroxy, ethoxy, —OCH₂CH₂OH, —CN, imidazolidin-2-one, phenyl or tetrazole wherein tetrazole is substituted with (C₁₋₄ alkyl);
R² is (C₁₋₆) alkyl;
R³ is hydrogen, (C₁₋₄) alkyl, or phenyl;
R⁴ and R⁵ are hydrogen;
R⁶ is (C₁₋₆) alkyl;
Alk is (C₁₋₄) alkylene;
X is CH₂, C(O), or S; and
Y is O or a bond.

8. The compound of claim 1, wherein

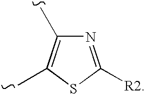

9. The compound of claim 8, wherein:
R¹ is hydrogen, or (C₁₋₄) alkyl;
R² is H, (C₁₋₄) alkyl, C(O)R⁶, or C(O)(OH)R⁶;
R³ is hydrogen, (C₁₋₄) alkyl, or phenyl;
R⁴ and R⁵ is hydrogen;
R⁶ is (C₁₋₄) alkyl;
Alk is (C₁₋₄) alkylene;
Y is O, or a bond; and
X is S, or CH₂.

10. The compound of claim 1, wherein

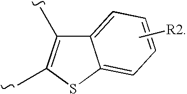

11. The compound of claim 10, wherein:
R¹ is hydrogen, or (C₁₋₄)alkyl;
R² is hydrogen;
R³ is phenyl or (C₁₋₄)alkyl;
R⁴ and R⁵ are independently selected from hydrogen or halogen;
Alk is (C₁₋₄) alkylene;
Y is O or a bond; and
X is CH₂.

12. The compound of claim 1, wherein the stereo configuration is “S” about the carbon of the piperazine group bound to Alk.

13. The compound of claim 12, wherein Alk is (C₂₋₄) alkylene and Y is O, S, or a bond.

14. The compound of claim 12, wherein Alk is methylene and Y is a bond.

15. The compound of claim 1, wherein the stereo configuration is “R” about the carbon of the piperazine group bound to Alk.

16. The compound of claim 15, wherein Alk is methylene and Y is O or S.

17-34. (canceled)

35. A pharmaceutical composition comprising an effective amount of a compound according to any claim 1 in association with a pharmaceutically acceptable carrier, diluent or excipient.

36-44. (canceled)

45. A method for treating a psychotic disorder, comprising administering to a mammal in need thereof an effective amount of a compound according to claim 1.

46. The method of claim 45, wherein the psychotic disorder is schizophrenia.

47. The method of claim 45, wherein the psychotic disorder is schizophreniform.

48. The method of claim 45, wherein the psychotic disorder is schizoaffective disorder.

49-69. (canceled)
